(19) United States
(12) Patent Application Publication
(10) Pub. No.: US 2026/0199254 A1
(43) Pub. Date: Jul. 16, 2026

(54) COMPOSITIONS AND METHODS OF USE THEREOF FOR INCREASING IMMUNE RESPONSES

(71) Applicant: UNIVERSITY OF GEORGIA RESEARCH FOUNDATION, INC., Athens, GA (US)

(72) Inventors: Jin XIE, Athens, GA (US); Zhengwei CAO, Athens, GA (US); Wei YANG, Athens, GA (US)

(21) Appl. No.: 19/132,384

(22) PCT Filed: Nov. 22, 2023
(86) PCT No.: PCT/US2023/080880

§ 371 Date: May 23, 2025

Related U.S. Application Data
(60) Provisional application No. 63/384,922, filed on Nov. 23, 2022.
(60) Provisional application No. 63/498,238, filed on Apr. 25, 2023.

(51) Int. Cl. A61K 9/51; A61K 35/14; A61K 39/00; A61K 39/39; A61K 45/06; A61K 47/68; A61K 47/69; A61P 35/00; C07K 16/28

(52) U.S. Cl. CPC A61K 9/5115; A61K 9/5146; A61K 35/14; A61K 39/39; A61K 45/06; A61K 47/68; A61K 47/69; A61P 35/00; C07K 16/2851; A61K 2039/55555; A61K 2039/55588

(57) ABSTRACT

Nanoparticles having a calcium core, such as calcium hydroxide (Ca(OH)₂) and calcium carbonate (CaCO₃) particles are provided. The nanoparticles can further include a shell such as one formed of silica or oleic acid. The nanoparticles can further include a coating, such as one formed of polyethylene glycol and optionally further including a lipid. The nanoparticles can further include a targeting agent, such as one that targets dendritic cells, T cells, or other immune cells. The nanoparticles can further include or otherwise be used in combination with an active agent, optionally selected from an antigen, chemotherapeutic drug, immune system modulator, or immune checkpoint modulator. Pharmaceutical compositions including the nanoparticles and methods of use thereof for increasing immune response, e.g., against cancer and infections are also provided.

### CROSS-REFERENCE TO RELATED APPLICATIONS

This application claims the benefit of and priority to U.S. Ser. No. 63/384,922 filed Nov. 23, 2022, and U.S. Ser. No. 63/498,238 filed Apr. 25, 2023, which are specifically incorporated by reference herein in their entireties.

### STATEMENT REGARDING FEDERALLY SPONSORED RESEARCH OR DEVELOPMENT

This invention was made with government support under CA257851, and CA247769 awarded by the National Institutes of Health. The government has certain rights in the invention.

### REFERENCE TO SEQUENCE LISTING

The Sequence Listing submitted as a text file named “UGA2023-053-03-PCT.xml,” created on Nov. 22, 2023, and having a size of 9,937 bytes is hereby incorporated by reference pursuant to 37 C.F.R § 1.52(e) (5).

### FIELD OF THE INVENTION

This invention is generally in the field of increasing immune responses, for example against cancer.

### BACKGROUND OF THE INVENTION

The role of the immune system in the fight against cancer is now well established. For example, dendritic cells (DCs) are the most effective type of antigen-presenting cells (APCs) and play important roles in protection against malignancies (Palucka, et al., Nature Reviews Cancer 2012, 12 (4), 265-277.). DCs constitutively sample their environment for antigens, process them, and migrate to the secondary lymphoid where they prime naïve T cells. During the process, DCs undergo maturation, marked by the upregulation of antigen-presenting molecules (major histocompatibility complexes MHC-I and MHC-II) and costimulatory molecules (e.g. CD80, CD86 and CD40) (Wculek, et al., Nature Reviews Immunology 2020, 20 (1), 7-24). DCs also secrete cytokines including interleukin 12 (IL-12) and type I interferons that shape T cell responses (Vignali, et al., Nature immunology 2012, 13 (8), 722-728, Parker, et al., Nature Reviews Cancer 2016, 16 (3), 131-144). As such, DCs function as an important bridge between the innate and adaptive immune responses.

However, tumor microenvironment (TME) is often rich in immunosuppressive factors, which negatively affect DC infiltration and antigen cross-presentation, curbing the immunity or inducing tolerance. The implications are broad because many cancer treatments rely on or benefit from DC-mediated immunity. For instance, radiotherapy (RT) and chemotherapy may induce immunogenic cell death (ICD), but as monotherapies they are often ineffective at eliciting a robust immunity. Immune checkpoint inhibitors may cause long-lasting remissions but many tumors are identified as immunologically “cold” and do not respond to immunotherapy (Binnewies, et al., Nature medicine 2018, 24 (5), 541-550). Some have explored immune modulators such as toll-like receptor (TLR) agonists, which may stimulate DCs and overcome the immunosuppressive TME (Pham, et al., Experimental & molecular medicine 2010, 42 (6), 407-419). However, issues including fast clearance, off-target toxicity, and suboptimal efficiency have limited their use in the clinic.

Additionally, cytotoxic T cells also play an important role. Cancer cells possess tumor-associated antigens (TAAs) that, like viruses and bacteria, can be recognized by the immune system and killed by cytotoxic T cells (CTLs) in an antigen-specific manner. However, solid tumors are often characterized by an immunosuppressive environment that inhibits T cell activation and proliferation or renders them anergic. Strategies, including immune checkpoint inhibitors (ICIs), are being developed to directly or indirectly enhance the functions of endogenous T cells. However, a significant proportion of patients do not respond to ICIs. Alternatively, antigen-specific T cells can be expanded or engineered outside the patient's body and reintroduced into the host. These include adoptive T cell transfer and CAR-T therapies, which have made significant progress and are entering the clinic. However, the efficacy of these therapies can still be limited by issues such as toxicity and the hostile tumor microenvironment. There is a need for new immunotherapy options that can be used either as a stand-alone treatment or in combination to augment existing immunotherapies.

Thus, there remains a need for compositions and methods to improve immune responses such as immune cell maturation and infiltration immune response therapies such as cancer therapy.

It is an object of the invention to provide compositions and methods to improve immune responses including, but not limited to, immune cell maturation and infiltration immune response therapies such as cancer therapy.

### SUMMARY OF THE INVENTION

Compositions and methods for improving responses such as immune cell maturation and infiltration in cancer therapy are disclosed.

The compositions include nanoparticles having a core including calcium. Examples include calcium hydroxide cores (herein CHNP), calcium carbonate (herein CCNP), calcium citrate (CaCit), calcium phosphate (Ca₃(PO₄)₂), CaCL₂, calcium sulfate (CaSO₄), CaC₂O₄, Ca(NO₃)₂, calcium silicate (Ca₂SiO₄), calcium fluoride (CaF₂), CaBr₂, and CaI₂.

The nanoparticles can include a shell, for example a silica or oleic acid shell.

In some forms the particles are hexagonal in shape. In, the experiments provided below, the CHNPs had an average diameter (the long diagonal of the hexagons) of about 219.9±17.8 nm, and the CCNPs had an average diameter of 150 to 160 nm, but other diameters are also contemplated. In some forms a silica shell has a thickness of about ~20 nm. In some embodiments, the nanoparticles further include a coating or other moiety such as polyethylene glycol (PEG) or lipid-PEG coating on, over, or incorporated with the shell.

In some embodiments, the nanoparticles include one or more targeting agents, such as a dendritic cell or T cell targeting agent. Such targeted particles can be referred to as AnCHNP, CCNP-Ab, etc. The targeting agent can be covalently associated with the nanoparticles, directly or indirectly via a linker. In some embodiments, the targeting agent targets one or more immune cells, e.g., dendritic cells, T cells (effector T cells (e.g., cytotoxic, helper, regulatory, or a combination thereof), memory T cells, Gamma-delta T cells (γδ T cells), Follicular helper T cells (Tfh), Natural killer T cells (NKT cells)), macrophages, natural killer cells, and/or neutrophils.

In some embodiments, the nanoparticles and/or formulations including them can further include a tumor-antigen and/or an immunomodulator such as an immune system modulator or immune cell modulator. In still another embodiment, the nanoparticle can include an additional cancer therapeutic such as an immune checkpoint inhibitor or chemotherapeutic agent. Some embodiments further include an adjuvant and/or antigen (e.g., an antigenic peptide).

Pharmaceutical compositions including an effective amount of the disclosed calcium particle compositions, and/or cells treated in vitro or ex vivo using the disclosed calcium particles, are also provided.

Methods of using the nanoparticle formulations, treated cells, and pharmaceutical compositions are also provided. The methods typically include increasing an immune response, for example, an activity of dendritic cells or T cells. The immune response can be induced by increased calcium signaling modulated by the calcium core particles.

The methods can be carried out in a subject in need thereof (i.e., in vivo), in vitro, or ex vivo. The subject can have a benign or malignant tumor or an infection. In some embodiments, the subject has cancer and optionally is undergoing cancer therapy, for example, vaccination, radiation therapy, chemotherapy, or immunotherapy. Exemplary cancers include, but are not limited to vascular, bone, muscle, bladder, brain, breast, cervical, colorectal, esophageal, kidney, liver, lung, nasopharangeal, pancreatic, prostate, skin, stomach, uterine, or germ cell cancer.

For example, a method of treating a subject in need thereof can include administering to the subject an effective amount of a disclosed nanoparticle formulation or ex vivo treated cells, preferably in a pharmaceutical composition, optionally further including one or more of an antigen, an immunomodulator (e.g., immune system modulator, immune cell modulator, etc.), an immune checkpoint inhibitor, and a chemotherapeutic agent.

### BRIEF DESCRIPTION OF THE DRAWINGS

FIGS. 1A-1I show synthesis and characterizations of AnCHNPs. FIG. 1A is a schematic illustration showing the nanoparticle synthesis, surface coating, and antibody-conjugation steps. FIG. 1B shows SEM images of CHNPs and SCHNPs. Scale bars, 200 nm. FIG. 1C shows TEM images of CHNPs (left) and SCHNPs (right). Scale bars represent 200 nm (black) and 100 nm (white) in length, respectively. FIG. 1D shows EDS elemental mapping which shows the core/shell structure of SCHNPs. Scale bar, 250 nm. FIG. 1E shows XRD spectra of SCHNPs, CHNPs, as well as Ca(OH)₂ reference (Ca(OH)₂ Ref). FIG. 1F shows EDS spectra of SCHNPs. FIG. 1G shows DLS spectra of CHNPs, PCHNPs and AnCHNPs, tested in water. FIG. 1H is a bar graph showing Zeta-potentials of CHNPs, SCHNPs, PCHNPs and AnCHNPs, measured in PBS (solutions). FIG. 1I is a scheme showing use of AnCHNPs to boost anti-cancer immunity. AnCHNPs are taken up by DCs and promote their maturation and migration to the secondary lymphoid organs such as tumor-draining lymph nodes (TDLNs), where they prime the native T cells. The activated DCs also secrete cytokines such as IL-12 that enhance the efficacy of effector T cells. The immunomodulation of AnCHNPs is most effective when they are used following radio- or chemo-therapy, which triggers the release of tumor antigens and possibly DAMPs.

FIGS. 2A-2H show the stability and intracellular degradation of AnCHNPs. FIG. 2A shows time-dependent released of Ca2+ from PCHNPs, tested in ammonium acetate buffer at pH 7.4 and 5.5. FIG. 2B shows TEM images showing the degradation the calcium core of PCHNPs in water. Scale bar: 100 nm. FIG. 2C is a bar graph showing DC uptake of AnCHNPs (Cy5-labeled, 5 μg/mL). Compared with PCHNPs, AnCHNPs showed significantly increased cellular uptake. *, p<0.05. FIG. 2D is a bar graph showing inhibition of DC uptake of AnCHNPs (Cy5-labeled, 5 μg/mL) by endocytosis inhibitors, including sodium azide (50 mM), dynasore (80 μM), nystatin (25 μM), and chlorpromazine (100 μM). *, p<0.05. FIG. 2E is a bar graph showing DC lysosomal pH changes, after incubation with AnCHNPs (5 or 10 μg/mL). FIG. 2F is a bar graph showing DC [Ca2+]int changes after cells being treated with AnCHNPs or CaCl2 (5 or 10 μg/mL). FIG. 2G is a bar graph showing DC [Na+]int changes, after incubation with AnCHNPs or CaCl₂) (5 or 10 μg/mL). FIG. 2H is a bar graph showing DC [K+]int changes, after incubation with AnCHNPs or CaCl₂) (5 or 10 μg/mL).

FIGS. 3A-3E show the impact of AnCHNPs on DC maturation and migration, tested with BMDCs after incubation with AnCHNPs or CaCl₂ (5 or 10 μg/mL). FIGS. 3A-3C show populations and mean fluorescence intensities (MFIs) of MHC-II+ and CD205+ in DCs. FIG. 3A shows quadrants showing the population changes. FIGS. 3B and 3C are histograms showing the fluorescence intensity (MFI) and population changes. FIG. 3D is a bar graph showing the impact of silica nanoparticles on DC maturation. FIG. 3E shows a transwell assay, tested with CFSE-labeled DCs. B16F10-OVA cells with (+) or without (−) 100 Gy pre-irradiation were seeded onto the bottom chamber. CFSE+ cells in the bottom at 24 h were quantified by flow cytometry. *, p<0.05; **, p<0.01; ***, p<0.001.

FIGS. 4A-4C show the impact of AnCHNPs on DC maturation, tested with BMDCs/B16F10-OVA (pre-irradiated, 100 Gy) co-cultures in the presence of AnCHNPs or CaCl₂) (5 or 10 μg/mL). FIG. 4A are quadrant graphs showing the changes of CD80⁺CD86⁺ and MHC-II⁺SIINFEKL-H-2Kb⁺ populations among DCs (CD11c⁺), analyzed by flow cytometry. In the control group, live B16F10-OVA were used in the co-culture and PBS was added into the incubation media. FIG. 4B includes bar graphs showing the frequencies of CD80⁺CD86⁺, CD40⁺, MHC-II⁺, and MHC-II⁺SIINFEKL-H-2Kb⁺ cells among DCs. FIG. 4C is bar graphs showing Pro-(IL-6, IL-12, and TNF-α) and anti-inflammatory (IL-10) cytokines in the supernatant of the co-cultures, analyzed by ELISA. *, p<0.05; **, p<0.01; ***, p<0.001.

FIGS. 5A-5F show studies evaluating DC activation by AnCHNPs at a molecular level. FIG. 5A is a cartoon showing the endocytosis of AnCHNPs leads to degradation in lysosome and Ca2+ release in cytosol. The increase of intracellular [Ca2+] activates pathways of transcription factors NF-κB and NFAT, which can elicit gene expression of activation markers and cytokine release. FIG. 5B is a heatmap of the top 10 most upregulated genes in AnCHNPs-treated BMDCs (vs Ctrl). FIG. 5C shows GO enrichment analysis of the top 10 GO terms resulting from upregulated DGEs in AnCHNPs-treated BMDCs (vs Ctrl). FIG. 5D shows GSEA analysis of enrichment plots for a priori gene sets for top four most upregulated pathways in in AnCHNPs-treated BMDCs (vs Ctrl). FIG. 5E shows Western blot examination of proteins of interest. BMDCs were treated with OVA (10 μg/mL) (Ctrl) or OVA (10 μg/mL)+AnCHNPs (5 μg/mL) (AnCHNPs) for 24 h then lysed for western blot analysis. Equal amounts of cell lysates were used for immunoblotting. NF-κB, phosphor-NF-κB, IκBαphosphor-IκBα, NFAT1 and Calcineurin were examined. GAPDH was used as the loading control of cytoplasmic protein. FIG. 5F is a bar graph showing expression of selected genes of cytokines and chemokines by RT-qPCR. *, p<0.05; **, p<0.01; ***, p<0.001. GSEA, gene set enrichment analysis; NES, normalized enrichment score.

FIGS. 6A-6D shows the impact of AnCHNPs on immune responses, tested in B16F10-OVA-tumor-bearing C57BL/6 mice. FIG. 6A is a scheme showing the experimental design. On Day 0, the animals received radiation (10 Gy) to tumors, followed by i.t. administration of AnCHNPs (200 μg/kg) (n=10). CaCl₂) plus RT and PBS plus RT were tested in control groups (n=10). Half of the animals from each group were euthanized on Day 3, while the rest were euthanized on Day 7. Tumor, TDLNs, spleen, and serum samples were collected for flow cytometry or ELISA analyses. FIG. 6B shows overall DC population in tumors on Day 3 and 7. FIG. 6C shows populations of CD86⁺CD80⁺, CD40⁺, MHC-II⁺, and MHC-II⁺SIINFEKL-H-2Kb⁺DCs in both tumors and TDLNs on Day 3 and 7. FIG. 6D shows T lymphocyte populations, including CTLs (CD45⁺CD3⁺CD8⁺), effector CTLs (IFN-γ⁺CD45⁺CD3⁺CD8⁺), and Tregs (CD45⁺CD3⁺CD4⁺Foxp3⁺), in both tumor and spleen on Day 3 and 7. CTL/Treg ratios were also calculated. FIG. 6E is a bar graph showing serum levels of cytokines, including IL-12, IFN-γ, IL-10, IL-1β, IL-6 and TNF-α, on Day 3 and 7. *, p<0.05; **, p<0.01; ***, p<0.001; ****, p<0.0001.

FIGS. 7A-7F show the therapeutic benefits of AnCHNPs when used in combination with RT, tested in both B16F10-OVA and MB49 tumor bearing C57BL/6 mice. FIGS. 7A-7D show the results of therapy studies with the B16F10-OVA model. FIG. 7A shows a scheme of the B16F10-OVA study. On Day 0 and 2, animals received radiation (10 Gy) applied to tumors, followed by i.t. administration of 200 μg/kg AnCHNPs (RT+AnCHNPs, n=5). PBS alone (PBS), PBS plus RT (RT+PBS), and AnCHNPs alone (AnCHNPs) were tested (n=5). For T cell depletion, anti-CD4 or anti-CD8 antibodies (i.p., 10 mg/kg, on Day 0 and 4) were administered in addition to the RT-and-AnCHNPs combination (RT+AnCHNPs+aCD4 and RT+AnCHNPs+αCD8, respectively; n=5). FIG. 7B shows average tumor growth, animal survival, and body weight curves. *, p<0.05; **, p<0.01; ***, p<0.001; ****, p<0.0001. FIG. 7C shows individual tumor growth curves. FIGS. 7D-7F shows therapy studies with the MB49 model. FIG. 7D shows a scheme of the study. On Day 0 and 2, animals received radiation (10 Gy) applied to tumors, followed by i.t. administration of AnCHNPs (200 μg/kg) (n=5). PBS alone (PBS), and PBS plus RT (RT+PBS), were tested (n=5). FIG. 7E shows average tumor growth, animal survival, and body weight curves. *, p<0.05. FIG. 7F shows individual tumor growth curves.

FIGS. 8A-8E show the results on evaluation of the benefits of AnCHNPs when used in combination with chemotherapy or immunotherapy. FIGS. 8A-8C show efficacy of dual therapy with AnCHNPs and carboplatin, tested in the B16F10-OVA model. FIG. 8A is a scheme of the therapy study. On Day 0 and 2, animals received carboplatin (i.p., 40 mg/kg on Day 0), followed by i.t. administration of 200 μg/kg AnCHNPs (carboplatin+AnCHNPs, n=5). PBS alone (PBS), and carboplatin alone (Carboplatin) were tested for comparison (n=5). FIG. 8B shows average tumor growth, animal survival, and body weight curves. *, p<0.05; **, p<0.01. FIG. 8C shows individual tumor growth curves. FIGS. 8D-8E show the efficacy of dual therapy with AnCHNPs and anti-PD-L1 antibodies, tested in the B16F10 model. FIG. 8D is a scheme showing the experimental design. On Day−2, 0, 2 and 4, animals received anti-PD-L1 antibodies (i.p., 10 mg/kg), followed by i.t. administration of 200 μg/kg AnCHNPs (aPD-L1+AnCHNPs, n=5). PBS alone (PBS), and anti-PD-L1 alone (aPD-L1) were tested for comparison (n=5). FIG. 8E shows average tumor growth, animal survival, and body weight curves. **, p<0.01.

FIG. 9A-9C show additional physiochemical characterizations of calcium nanoparticles, including calcium hydroxide nanoparticles (CHNPs), silica-coated calcium hydroxide nanoparticles (SCHNPs), and PEGylated calcium hydroxide nanoparticles (PCHNPs). FIG. 9A shows FT-IR spectra of CHNPs, SCHNPs, and PCHNPs. APTES (3-aminopropyl)triethoxysilane), which was used in silica coating, and PEG-diacid, which was used in surface PEGylation, were also analyzed. FIG. 9B shows EDS analysis of CHNPs. The Ca-to-O molar ratio was ~1:2. FIG. 9C shows Zeta potentials of CHNPs, SCHNPs, PCHNPs, and AnCHNPs, tested in PBS.

FIGS. 10A-10D show calcium release in solutions and in vitro. Quantification of calcium levels in solutions was achieved using an ion-selective electrode. In vitro quantification was based on a chromogenic complex formed between calcium ions and 0-cresolphthalein, this chromophore was measured at OD=575 nm. FIG. 10A is a standard calibration curve for potentiometry measurements, established with calcium salt (CaCl₂), 150 ppm and 2000 ppm) with known concentrations. FIG. 10B shows time-dependent Ca²⁺ release from CHNPs, tested in ammonium acetate buffers at pH 7.4 and 5.5. FIG. 10C is a bar graph showing cytotoxicity of AnCHNPs, CaCl₂), and PEGylated silica nanoparticles, tested with BMDCs using ATPlite-1step luminescence assay. FIG. 10D contains bar graphs showing lysosomal pH changes after cells being treated with AnCHNPs (5 and 10 μg/mL), measured with BMDCs using LysoSensor™ Yellow/Blue DND-160 (PDMPO), which has predominantly yellow fluorescence (440 nm) in acidic organelles, and in less acidic organelles it has blue fluorescence (540 nm). Dual-emission measurements may permit ratio imaging of the pH in acidic organelles.

FIGS. 11A-11B show the impact of AnCHNPs on immune responses of DCs and T cells, tested in B16F10-OVA-tumor-bearing C57BL/6 mice. FIG. 11A shows populations of CD86+CD80+, CD40+, MHC-II+, and MHC-II+SIINFEKL-H-2Kb+ DCs in spleen on Day 3 and 7. FIG. 11B shows T lymphocyte populations, including CTLs (CD45+CD3+CD8+), effector CTLs (IFN-γ+CD45+CD3+CD8+), and Tregs (CD45+CD3+CD4+Foxp3+), in TDLN on Day 3 and 7. CTL/Treg ratios were also calculated. *: p<0.05; **: p<0.01; ***: p<0.001; ****: p<0.0001.

FIG. 12 shows the impact of AnCHNPs on antigen specific cellular immunity. Splenocytes taken from the AnCHNPs-treated group were co-incubated with B16F10-OVA cells for 6 h ex vivo, IFN-γ⁺ CTL frequency was measured by flow cytometry. Splenocytes from PBS- and CaCl₂)-treated groups were also studied.

FIG. 13 shows flow cytometry gating strategy for the DC migration study.

FIG. 14 shows flow cytometry gating strategy for the in vivo immune profiling study that examines populations of DCs in tumors and TDLNs.

FIG. 15 shows flow cytometry gating strategy for the in vivo immune profiling study that examines populations of lymphocytes in tumors and spleen.

FIGS. 16A-16N show the results of nanoparticle synthesis and characterization. FIG. 16A is a TEM image of CaCO₃ nanoparticles. FIG. 16B is Zoom-in TEM image of CaCO₃ nanoparticles; scale bar: 100 nm. FIG. 16C is an SEM image of CaCO₃ nanoparticles. FIG. 16D is a plot showing size distribution of CaCO₃ nanoparticles based on TEM results. FIG. 16E is a TEM image of CaCO₃@OA nanoparticles. FIG. 16F is a Zoom-in TEM image of CaCO₃@OA nanoparticles; scale bar, 100 nm. FIG. 16G is an SEM image of CaCO₃@OA nanoparticles. FIG. 16H is a plot showing size distribution of CaCO₃@OA nanoparticles based on TEM results. FIG. 16I is a TEM Energy Dispersive X-ray Spectroscopy (EDX) plot of CaCO₃ nanoparticles. FIG. 16J is an X-ray Diffraction (XRD) of CaCO₃ nanoparticles (upper plot) and bulk CaCO₃ (low plot). FIG. 16K is an Fourier-transform infrared spectroscopy (FT-IR) plot comparing OA, CaCO₃@OA, and CaCO₃ nanoparticles. FIG. 16L is a plo showing Zeta potentials of CCNP and CCNP-Ab, measured in HEPES buffer (pH=7.4). FIG. 16M is a plot showing DLS measurement of CaCO₃ nanoparticles (in ethanol), CaCO₃@OA (in hexane), CCNP (in HEPES), and CCNP-Ab (in HEPES). FIG. 16N is a plot showing calcium release from CCNP-Ab, tested at pH=7.4 and 5.0 at room temperature.

FIGS. 17A-17J show the results of in vitro studies. FIG. 17A is a plot showing cytotoxicity of PMA@CCNP-Ab and CaCl₂), measured in EL4 cells. Nanoparticle dose was based on equivalent calcium concentrations. FIG. 17B is an IC50 viability curve based on viability data from FIG. 17A. FIG. 17C is a plot showing cellular uptake data. PMA@CCNP and PMA@CCNP-Ab were labeled with Cy5. The mean fluorescence intensity (MFI) of Cy5 was measured after 24 h co-culture with EL4. FIG. 17D is a plot showing changes in intracelluar calcium levels. Fluo-3AM was used as a calcium indicator. FIGS. 17E and 17F are images of Western blotting to analyze the effects of PMA@CCNP-Ab on NF-κB (FIG. 17E) and NFAT (FIG. 17F) pathways. FIGS. 17G and 17H are each a series of plots showing immune profiling of OT-1 CTLs after cells were treated with PMA@CCNP-Ab for 48 h (FIG. 17G) and 72 h (FIG. 17H). FIG. 17I is a plot showing IFN-γ secretion from OT-1 cells (primed with irradiated B16-OVA) after being treated with PMA@CCNP-Ab and control groups. FIG. 17J is a plot showing IL-2 secretion from OT-1 cells (primed with irradiated B16-OVA) after being treated with PMA@CCNP-Ab and control groups.

FIGS. 18A-18D show evaluation of immunostimulatory effects of PMA@CCNP-Ab in vivo. B16-OVA-tumor-bearing C57BL/6 mice were irradiated (15 Gy), followed by i.t. injection of PMA@CCNP-Ab on Day 2, 5, and 8. Flow cytometry was performed on samples from tumor (FIG. 18A), spleen (FIG. 18B), and lymph node (FIG. 18C) harvested on Day 15. FIG. 18D is a dot plot showing the results of co-culture of splenocytes and B16-OVA cancer cells and evaluation of activated CTLs (CD8⁺IFN-γ⁺) using flow cytometry.

FIGS. 19A-19C show evaluation of the therapeutic benefit of PMA@CCNP-Ab in vivo in B16 tumor-bearing C57BL/6 mice. PMA@CCNP-Ab nanoparticles were injected i.t. on day 0, 1 and 3. PBS or CaCl₂) salt was injected for comparison. In addition, anti-CD8 antibodies were injected in addition to PMA@CCNP-Ab to evaluate the impact of CTLs on the therapeutic effects. FIG. 19A is an animal survival curve. FIG. 19B is a tumor growth curve. FIG. 19C is a series of plots showing individual tumor growth curves.

### DETAILED DESCRIPTION OF THE INVENTION

The disclosed compositions are based at least on the discovery that safe and effective calcium modulators that can boost immune cell activation, e.g., DC-mediated and/or T cell-mediated anticancer immunity.

Ca²⁺ as a second messenger plays an important role in DC maturation and migration. Resting, immature DCs maintain a low-level of cytosolic calcium or [Ca²⁺]ᵢₙₜ. Cytokines, pathogen-associated molecular patterns, or damage-associated molecular patterns may bind to DC receptors and trigger an increase of [Ca²⁺]ᵢₙₜ, which in turn activate signaling cascades that ultimately induce costimulatory and antigen-presenting molecules (Shumilina, et al., American Journal of Physiology-Cell Physiology 2011, 300 (6), C1205-C1214). [Ca²⁺]ᵢₙₜ is tightly regulated by calcium-selective ion channels and transporters on the plasma membrane, endoplasmic reticulum, and the inner mitochondrial membrane. Previously, calcium ionophores (e.g. ionomycin) are shown to be able to elevate [Ca²⁺]ᵢₙₜ and activate DCs in a laboratory setting (Liu, et al., Journal of Biological Chemistry 1978, 253 (17), 5892-5894). However, these ionophores lack specificity for DCs, and may cause toxicity when administered systemically (Jiang, et al., Nature 1995, 375 (6527), 151-155). Moreover, DC maturation and activation requires an endured increase of [Ca²⁺]ᵢₙₜ (Santegoets, et al., Journal of leukocyte biology 2008, 84 (6), 1364-1373), which is challenging or not possible with small molecule ionophores that are rapidly cleared after injection.

Calcium also plays a central role in T cell activation as a second messenger. Calcium signaling begins with stimulation of the TCR pathway and ultimately leads to activation of the transcription factor NFAT through activation of the calcium-sensitive phosphatase calcineurin.

There is an unmet need for safe and effective calcium modulators that can boost DC-mediated and T cell-mediated anticancer immunity.

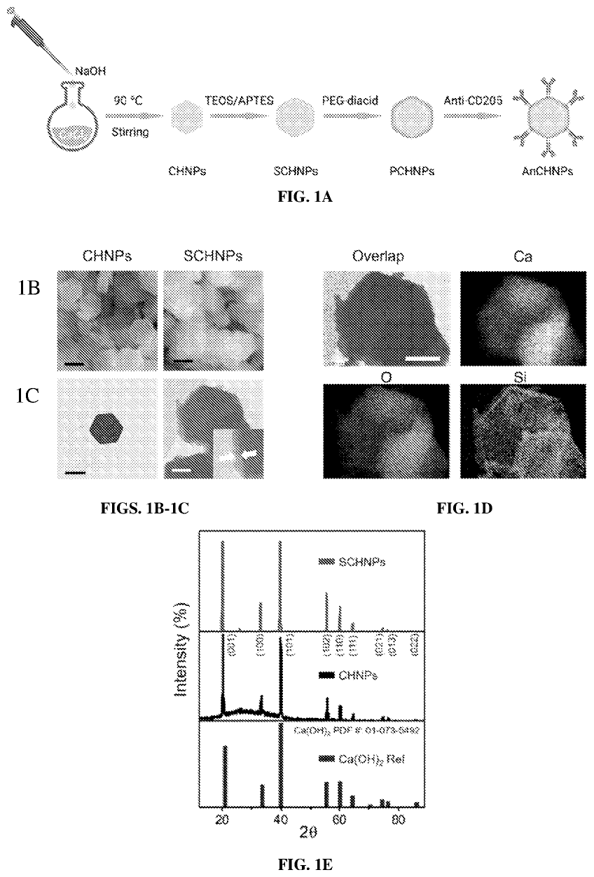

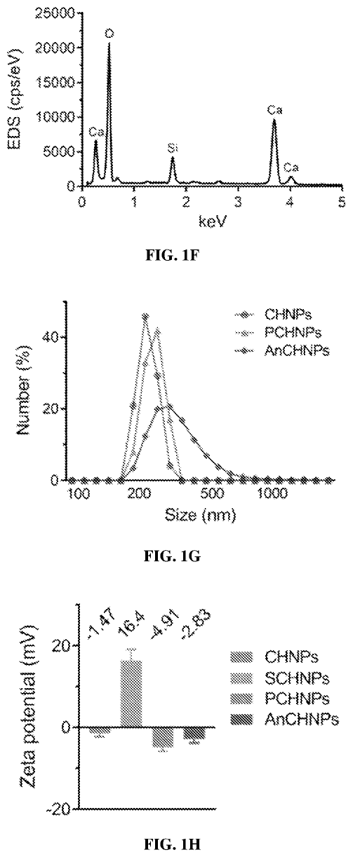

Herein, the Examples demonstrate the use of calcium nanoparticles as a DC-targeted immunomodulator. Briefly, Ca(OH)₂ nanoparticles were synthetized through co-precipitation and coupled with antibodies specific to anti-CD205 (a.k.a. DEC205), a type I integral membrane protein expressed primarily on DCs (Jiang, et al., Nature 1995, 375 (6527), 151-155). The resulting antibody-conjugated calcium hydroxide nanoparticles (AnCHNPs) were shown to be selectively taken up by DCs and released calcium therein to enable a sustained increase of [Ca²⁺]ᵢₙₜ. This increase in [Ca²⁺]ᵢₙₜ promoted DC maturation, migration, cross-presentation, in turn augmenting T cell immunity (FIG. 1I). These results were demonstrated in vitro with bone marrow derived dendritic cells (BMDCs), and in vivo using AnCHNPs as an adjuvant in combination with RT, immunotherapy, or chemotherapy. The Examples demonstrate that CHNPs including a DC targeting moiety is useful as an adjuvant in combination with RT, immunotherapy, or chemotherapy.

Additional Examples show that T cells efficiently internalized calcium nanoparticles, e.g., PMA@CCNP-Ab, resulting in increased intracellular calcium levels. Delivery of calcium and PMA to T cells promoted their activation as evidenced by increased expression or secretion of CD69, IFN-γ, and TNF-α. This was observed both in the EL4 cell line and in primary T cells from OT1 mice. In vivo testing in B16-OVA tumor-bearing C57/BL6 mice showed that PMA@CCNP-Ab resulted in enhanced tumor infiltration by cytotoxic T cells and increased CTL/Treg ratios. Therapeutic benefits associated with PMA@CCNP-Ab's ability to enhance T cell activation were observed. In addition, PMA@CCNP-Ab can be used to enhance cell-based therapies, including adoptive T-cell transfer and CAR-T therapies.

### I. DEFINITIONS

The term “nanoparticle” refers to any particle having a diameter greater than 1 nm and less than 1000 nm.

The term “targeting agent” and “targeting moiety” refers to a chemical compound that can direct a nanoparticle to a site on a selected cell or tissue type, can serve as an attachment molecule, or serve to couple or attach another molecule. The term “direct,” as relates to chemical compounds, refers to causing a nanoparticle to preferentially attach to a selected cell or tissue type. This targeting agent, generally binds to its target with high affinity and specificity.

The terms “treatment” and “treating”, as used herein, refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “tumor” or “neoplasm” refers to an abnormal mass of tissue containing neoplastic cells. Neoplasms and tumors may be benign, premalignant, or malignant.

The term “cancer” or “malignant neoplasm” refers to a cell that displays uncontrolled growth, invasion upon adjacent tissues, and often metastasis to other locations of the body.

The term “individual,” “subject,” and “patient” are used interchangeably to refer to any individual who is the target of administration or treatment. The subject can be a vertebrate, for example, a mammal. Thus, the subject can be a human or veterinary patient.

The term “therapeutically effective” means that the amount of the composition used is of sufficient quantity to ameliorate one or more causes or symptoms of a disease or disorder. Such amelioration only requires a reduction or alteration, not necessarily elimination. A therapeutically effective amount of a composition for treating cancer is preferably an amount sufficient to cause tumor regression or to sensitize a tumor to radiation or chemotherapy.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. COMPOSITIONS

### A. Core Composition

The compositions include particles, typically nanoparticles, that have a core including calcium. The particles are designed to enter cells and release calcium ions (e.g., Ca²⁺) within the cells. The particles typically include one or more of the following features: Controlled calcium release. T-cell activation utilizes a sustained increase in intracellular calcium concentration ([Ca²⁺]ᵢₙₜ) and increases of [Ca²⁺]ᵢₙₜ activates signaling cascades that ultimately induce costimulatory and antigen-presenting molecules in dendritic cells. Achieving a sustained increase in [Ca₂₊]ᵢₙₜ is difficult with calcium salts (due to the ion-impermeable plasma membrane) or bare calcium nanoparticles (due to rapid particle dissolution in the tumor microenvironment). To solve this problem, a shell and/or coating layer can be used that prevents nanoparticles from rapid degradation, allowing nanoparticles to enter cells through endocytosis and gradually release calcium ions inside cells.

Low toxicity: Unlike cytokine or interferon-based immunomodulators, the calcium nanoparticles can have low toxicity and can be administered repeatedly without causing systemic toxicity. After treatment, the nanoparticles can degrade to Ca²⁺ and other partnering components such as CO₃²⁻, which are safely excreted, metabolized or absorbed by the host.

Targeted delivery: The nanoparticles can be conjugated with targeting ligands to facilitate targeted delivery of calcium and/or loaded with active agents e.g., antigen and/or PKC antagonists.

Unique mechanism of action: Cell activation can be suppressed or blocked at multiple stages, dampening cellular immunity. In the disclosed approach, calcium delivery can bypasses upstream signaling, which is believed to allow cell activation even in immunosuppressive environments.

In some embodiments, the particles have a calcium hydroxide core. Also referred to as Ca(OH)₂ nanoparticles and CHNPs, the experiments below show such particles can be synthesized through a co-precipitation method using CaCl₂) and NaOH as precursors.

In some embodiments, the particles have a calcium carbonate core. Also referred to CaCO₃ and CCNPs, the experiments below show such particles can be synthesized through a co-precipitation method with calcium chloride and ammonium bicarbonate precursors.

Other calcium core particles are also contemplated and include, but are not limited to calcium citrate (CaCit), calcium phosphate (Ca₃(PO4)₂), CaCL₂, calcium sulfate (CaSO₄), CaC₂O₄, Ca(NO₃)₂, calcium silicate (Ca₂SiO₄), calcium fluoride (CaF₂), CaBr₂, and CaI₂, each of which may also be specifically excluded.

Exemplary methods of making calcium core particles are discussed in the experiments below.

See, also e.g., Rimsueb, et al., ACS Omega, 5, 13, 7418-7423 (2020) doi.org/10.1021/acsomega.0c00032; Khalifehzadeh and Arami, Advances in Colloid and Interface Science, Volume 279, May 2020, 102157, doi.org/10.1016/j.cis.2020.102157; Leukel, et al., Langmuir, 34, 24, 7096-7105 (2018) doi.org/10.1021/acs.langmuir.8b00927; Li, et al., Theranostics. 2016 Oct. 7; 6(13):2380-2393. doi: 10.7150/thno. 15914; Putnis, et al., Crystallization via Nonclassical Pathways Volume 2: Aggregation, Biomineralization, Imaging & Application, Chapter 1 pp 1-35, ACS Symposium Series Vol. 1383 (2021). DOI: 10.1021/bk-2021-1383.ch001; Jiang, et al., ACS Appl. Nano Mater. 2022, 5, 9, 13069-13077, doi.org/10.1021/acsanm.2c02852, WO 2020/150623, and WO 2023/039415.

The disclosed particles are typically nanoscale in size, for example, having a diameter of 10 nm up to, but not including, about 1 micron. However, it will be appreciated that in some embodiments, and for some uses, the particles can be smaller or larger (e.g., microparticles, etc.). Although many of the compositions disclosed herein are referred to as nanoparticle compositions, it will be appreciated that in some embodiments and for some uses the particle can be somewhat larger than nanoparticles. For example, compositions can also include particles having a diameter of between about 1 micron to about 1000 microns. Such compositions can be referred to as microparticle compositions. Thus, all the particle compositions provided here can be microparticles, but are typically more preferably nanosized nanoparticles.

Nanoparticles are often utilized for intratissue applications and penetration of cells. Thus, in some embodiments, the particles are nanoparticles that have any diameter from 10 nm up to about 1,000 nm, or any subrange or specific integer therebetween. For example, the nanoparticles can have a diameter from 10 nm to 900 nm, from 10 nm to 800 nm, from 10 nm to 700 nm, from 10 nm to 600 nm, from 10 nm to 500 nm, from 20 nm from 500 nm, from 30 nm to 500 nm, from 40 nm to 500 nm, from 50 nm to 500 nm, from 50 nm to 400 nm, from 50 nm to 350 nm, from 50 nm to 300 nm, or from 50 nm to 200 nm, from 10 nm to 100 nm. For example, in some embodiments, the particles are about 15 nm, 25 nm, 60 nm, 100 nm, 150 nm, 200 nm, 250 nm, 300 nm, or any other integer value or range of values between 1 nm and 1000 nm inclusive. In some embodiments the nanoparticles can have a diameter less than 400 nm, less than 300 nm, or less than 200 nm. For example, the nanoparticle can have a diameter from between 50 nm and 300 nm.

The disclosed sizes can be the particle size with or without a shell and/or coating. Thus, in some embodiments, the sizes are the average diameters of the particle core.

In one example, the average diameters of the core of the nanoparticles are between about 15 nm and about 800 nm, or between about 20 nm and about 500 nm, or between about 50 nm and about 350 nm, or any subrange or specific integer there between. In some embodiments, the average diameters of the nanoparticles are about 100 nm or 150 nm or 200 nm to about 200 nm or 250 nm or 300 nm.

Particles size can be measured or determined by, for example, dynamic light scattering, electronic microscopy such as scanning electron microscopy (SEM), and transmission electron microscopy (TEM).

In some embodiments the particles in a particle composition are monodispersed. In some embodiments, the particles in a particle composition are of various sizes (i.e., polydispersed).

### B. Shell

In some embodiments, the calcium core is surrounded by a shell. Shells can be or include metal-organic frameworks, protein shells (e.g., ferritin, albumin, and virus-like particles), noble metals (Au, Ag, Pt, et.), carbon, etc. Shells can be formed from, for example, silica, mesoporous silica, carbon; sulfides such as ZnS, CoS, CuS, Cu2S, FeS, MoS, Al2S3, Y2S3, and MnS, etc.; oxides such as Fe3O4, Fe₂O₃, Gd2O3, TiO2, Al2O3, MnO2, etc.; fluorides such as NaYF4, YF3, LaF3, CeF3, PrF3, and GdFe3; fatty acids such as oleic acid, myristic acid, palmitic acid, palmitoleic acid, stearic acid, oleic acid, linoleic acid, arachidic acid, eicosapentaenoic acid (EPA), docosahexaenoic acid (DHA); Alkyl amines such as octylamine, nonylamine, decylamine, undecylamine, laurylamine, tridecylamine, tetradecylamine, pentadecylamine, hexadecylamine, heptadecylamine, octadecylamine, oleylamine; MgO, CuO, and ZnO.

In a preferred embodiment, the shell is formed of silica. In some of the experiments below, the silica shell was added to core particles using a mixture of tetraethyl orthosilicate (TEOS) and (3-aminopropyl)triethoxysilane) (APTES) as silane precursors so that the resulting nanoparticles present amine groups on the surface.

In some embodiments, the shell is formed of oleic acid. In some of the experiments below, the oleic acid shell was added to core particles by dispersing particles in a mixture of ethanol and oleic acid.

In some embodiments, a protective shell is added to reduce, prevent, or otherwise delay degradation of the particles. Preferably, the shell is composed of material(s) that is/are low-toxic, stable at neutral pH, and/or biodegradable. In some embodiments, the shell is hydrophobic.

### C. Coating

To further enhance the nanoparticles, a coating can be added. In some embodiments, the coating can improve dispersion in aqueous solutions and/or delay core release and/or improve half-life. Such coating is preferably applied over or integrated with a shell, but application directly over the core (e.g., in the absence of a shell), is also contemplated.

In some of the experiments below, PEG-diacid coating was added to silica-shelled CHNPs by dispersing particles in a mixture of dimethyl sulfoxide (DMSO) and PEG-diacid.

In some of the experiments below, 1,2-distearoyl-sn-glycero-3-phosphoethanolamine-N-[carboxy (polyethylene glycol)-2000] (DSPE-PEG-COOH) was added to oleic acid-shelled CaCO₃ (CaCO₃@OA) particles hydrophobic interactions by dispersing the particles in a mixture of hexane and DSPE-PEG-COOH.

Thus, the particles optionally, but preferably, include a coating. Also referred to herein as a layer, or external layer, the coating/layer is typically over the core and optionally, but preferably, over or integrated with the shell of the particles. In some embodiments, the coating enhances the particles' compatibility with aqueous solutions. Additionally or alternatively, the coating can be added to extend the half-lives of the nanoparticle in aqueous environments and/or improve nanoparticle uptake by cells.

### 1. Composition of the Coating

The coating can be composed of, for example, polar or non-polar polymers and co-polymers, peptides, proteins, lipids, silica, metal oxides, or combinations thereof. In some embodiments, the coating is composed of conjugates or fusions of two or more of the foregoing alone or in further combination with one or more active agents and/or targeting moieties.

For example, in some embodiments, the thickness of the coating, inclusive or exclusive of a shell, ranges from 1 nm to 200 nm, or 10 nm to 100 nm, or 25 nm to 75 nm inclusive, or any subrange or specific integer therebetween, such as 50 nm.

While PEG is a preferred polymer base for forming a coating, optionally with additional moieties such as charge modifying moieties (e.g., carboxyl groups) and/or target moieties such as antibodies or others mentioned herein or elsewhere, other coatings are also contemplated, and examples are discussed below.

a. Polymers

In some embodiments, the layer or coating around the particles is formed of one or more polymers. The polymer can be polar, non-polar, or amphiphilic, and can be a single polymer or a copolymer. Polymer refers to a molecular structure including one or more repeat units (monomers), connected by covalent bonds. A biocompatible polymer refers to a polymer that does not typically induce an adverse response when inserted or injected into a living subject. A copolymer refers to a polymer formed of two or more different monomers. The different units may be arranged in a random order, in an alternating order, or as a “block” copolymer, i.e., including one or more regions each including a first repeat unit (e.g., a first monomer or block of monomers), and one or more regions each including a second repeat unit (e.g., a second block), etc. Block copolymers may have two (a diblock copolymer), three (a triblock copolymer), or more numbers of distinct blocks.

In preferred embodiments, particularly where the surface in contact with the coating (e.g., the core or the shell, etc.) is hydrophobic, the coating is formed of an amphiphilic molecule. The term “amphiphilic” refers to a molecule that has both a polar portion and a non-polar portion. In some embodiments, the polar portion (e.g., a hydrophilic portion such as a hydrophilic polymer) is soluble in water, while the non-polar portion (e.g., a hydrophobic portion such as a hydrophobic polymer) is insoluble in water. The polar portion may have either a formal positive charge, or a formal negative charge. Alternatively, the polar portion may have both a formal positive and a negative charge, and be a zwitterion or inner salt.

The hydrophilic portion of the amphiphilic material can form a corona around the particle that increases the particle's solubility in aqueous solution. In a particular embodiment, the amphiphilic material is a hydrophobic, biodegradable polymer terminated with a hydrophilic block.

The hydrophilic portion and hydrophobic portion can be biocompatible hydrophilic and hydrophobic polymers respectively. Exemplary biocompatible polymers include, but are not limited to, polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polylactides, polyglycolides, polysiloxanes, polyurethanes and copolymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt; polyacrylic acid polymers such as polymers of acrylic and methacrylic esters such as poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyalkylenes such as polyethylene, polypropylene poly(ethylene glycol), poly(ethylene oxide), and poly(ethylene terephthalate), poly(vinyl alcohols), poly(vinyl acetate), poly vinyl chloride polystyrene and polyvinylpryrrolidone, derivatives thereof, linear and branched copolymers and block copolymers thereof, and blends thereof.

Other exemplary biodegradable polymers include, but are not limited to, polyesters, polydopamine, poly(ortho esters), poly(ethylene imines), poly(caprolactones), poly(hydroxybutyrates), poly(hydroxyvalerates), polyanhydrides, poly(acrylic acids), polyglycolides, poly(urethanes), polycarbonates, polyphosphate esters, polyphosphazenes, derivatives thereof, linear and branched copolymers and block copolymers thereof, and blends thereof. In particularly preferred embodiments the co-polymer include one or more biodegradable hydrophobic polyesters such as poly(lactic acid), poly(glycolic acid), and poly(lactic-co-glycolic acid), and/or these polymers conjugated to polyalkylene oxides such as polyethylene glycol or block copolymers such as the polypropylene oxide-polyethylene oxide PLURONICS®.

The molecular weight of the biodegradable oligomeric or polymeric segment or polymer can be varied to tailor the properties of the polymer.

In some embodiments, the hydrophilic polymers or segment(s) or block(s) include, but are not limited to, homo polymers or copolymers of polyalkene glycols, such as poly(ethylene glycol), poly(propylene glycol), poly(butylene glycol), and acrylates and acrylamides, such as hydroxyethyl methacrylate and hydroxypropyl-methacrylamide.

The hydrophobic portion of amphiphilic materials can provide a non-polar polymer matrix coating for loading non-polar drugs.

b. Lipids

The coating can be, or include, one or more lipids. Lipids and other components useful in preparing the disclosed nanoparticle compositions having a lipid-based coating are known in the art. Suitable neutral, cationic and anionic lipids include, but are not limited to, sterols and lipids such as cholesterol, phospholipids, lysolipids, lysophospholipids, and sphingolipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; phosphoethanolamines such as 1,2-distearoyl-sn-glycero-3-phosphoethanolamine (DSPE), 1,2-diacyl-sn-glycero-3-phosphoethanolamine, including, but not limited to, 1,2-dioleylphosphoethanolamine (DOPE), 1,2-dihexadecylphosphoethanolamine (DHPE); and phophatidylcholines such as 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

The lipid can be a sphingomyelin metabolites such as, without limitation, ceramide, sphingosine, or sphingosine 1-phosphate.

Exemplary catonic lipids include, but are not limited to, N-[1-(2,3-dioleoyloxy) propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy) propyl]-N,N-dimethyl amine (DODAP), 1,2-diacyloxy-3-dimethylammonium propanes, N-[1-(2,3-dioleyloxy) propyl]-N,N,N-trimethylammonium chloride (DOTMA), 1,2-dialkyloxy-3-dimethylammonium propanes, dioctadecylamidoglycylspermine (DOGS), 3-|N—(N′,N′-dimethylamino-ethane)carbamoyl|cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9 (Z)-octadecenoyloxy)ethyl]-2-(8 (Z)-heptadecenyl-3-(2-hydroxyethyl) imidazolinium chloride (DOTIM), and 1-[2-(hexadecanoyloxy)ethyl]-2-pentadecyl-3-(2-hydroxyethyl) imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids can be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine).

A sterol component may be included to confer a physicochemical and biological behavior. Such a sterol component may be selected from cholesterol or its derivative e.g., ergosterol or cholesterolhemisuccinate.

The coating can include a single type of lipid, or a combination of two or more lipids, or one or more lipids in combination with other materials.

c. Polyethers and Polyquaterniums

The coating can be, or include, a polyether. Exemplary polyethers include, but are not limited to, oligomers and polymers of ethylene oxide. In preferred embodiments, the polyether is a Polyethylene glycol (PEG). PEGs are prepared by polymerization of ethylene oxide and are commercially available over a wide range of molecular weights from 300 g/mol to 10,000,000 g/mol, and can have branched, star, or comb geometries. The numbers that are often included in the names of PEGs indicate their average molecular weights (e.g. a PEG with n=9 would have an average molecular weight of approximately 400 daltons, and would be labeled PEG 400.) Most PEGs include molecules with a distribution of molecular weights (i.e., they are polydisperse). The size distribution can be characterized statistically by its weight average molecular weight (Mw) and its number average molecular weight (Mn), the ratio of which is called the polydispersity index (Mw/Mn). Mw and Mn can be measured by mass spectrometry. In some embodiment the PEG is an amino (polyethylene glycol) (also referred to as a PEG amine).

In some embodiments, the PEG or PEG amine is up about 25,000, or more. In some embodiments, the PEG or PEG amine is about PEG 350 to about PEG 25,000, or about PEG 350 to about PEG 20,000. In some embodiments, the PEG or PEG amine is about PEG 350 to about PEG 5000, or between about PEG 750 and about PEG 5000, or between about PEG 1000 and PEG 3000. In a particular embodiment, the PEG is PEG 2000.

In particular embodiments, the coating is a polyether-lipid (e.g., phospholipid) conjugate coating.

In some embodiments, the coating includes or is formed of one or more polyquaterniums. Polyquaternium is the International Nomenclature for Cosmetic Ingredients designation for several polycationic polymers that are used in the personal care industry. Polyquaternium is a neologism used to emphasize the presence of quaternary ammonium centers in the polymer. INCI has approved at least 40 different polymers under the polyquaternium designation. Different polymers are distinguished by the numerical value that follows the word “polyquaternium”, and include, e.g., polyquaternium-1 through polyquaternium-20, polyquaternium-22, polyquaternium-24, polyquaternium-27 through polyquaternium-37, polyquaternium-39, and polyquaternium-42 through polyquaternium-47. In particular embodiments, the polyquaternium is polyquaternium-7, -10, or -30.

d. Charge Modifying Moieties

The coating and/or shell can include a charge modifying moiety, e.g., at the terminal end of some or all of molecules from which it is formed. For example, the can be formed of a material having the structure A-X where A is a hydrophobic molecule or hydrophobic polymer, and X is a terminal moiety that imparts a charge, e.g., a negative charge to the particle. The material can have the structure A-B-X where A is a hydrophobic molecule or hydrophobic polymer, B is a hydrophilic molecule or hydrophilic polymer, and X is a terminal moiety that imparts a chart, e.g., a negative charge. In some embodiments, the shell includes an anionic lipid; a negatively charged moiety attached to a cationic, neutral lipid, an anionic lipid, and/or to a linker such as PEG; or a combination thereof. In particular embodiments, the terminal moiety is an acidic group or an anionic group pendant on a hydrophilic group (PEG). Acidic groups include, for example, carboxylic acids, protonated sulfates, protonated sulfonates, protonated phosphates, singly- or doubly protonated phosphonates, and singly- or doubly protonated hydroxamate. Anionic groups include, for example, carboxylates, sulfates, sulfonates, singly- or doubly deprotonated phosphate, singly- or doubly deprotonated phosphonate, and hydroxamate. Positive charge moieties include, but are not limited to, primary, secondary, and tertiary amines, guanidines, imines, and imidazoles, etc.

In some embodiments, the coating is formed partly or completely of a material including a lipid (e.g., a phospholipid such as DSPE conjugated to PEG conjugated to a negatively charged terminal moiety such as COOH).

### D. Targeting Agents and Other Functional Molecules

Functional molecules can be associated with, linked, conjugated, or otherwise attached directly or indirectly to the disclosed particles. One class of functional elements is targeting molecules.

For example, it is also possible to inject the disclosed particles systemically and rely on either passive or active targeting of NPs to target tissue. Thus, in some embodiments, the particles include a targeting agent, most typically conjugated to one or more components of the coating. The targeting moiety can specifically recognize and bind to a target molecule specific for a cell type, a tissue type, or an organ. The target molecule can be or target a cell surface polypeptide, lipid, or glycolipid or a ligand thereof. The targeting agent can be covalently associated with the nanoparticles, directly or indirectly via a linker.

### 1. Exemplary Forms of Targeting Agents

Targeting molecules can be proteins, peptides, nucleic acid molecules, saccharides or polysaccharides that bind to a receptor or other molecule on the surface of a targeted cell. The degree of specificity and the avidity of binding to the graft can be modulated through the selection of the targeting molecule. For example, antibodies are very specific. These can be polyclonal, monoclonal, fragments, recombinant, or single chain, many of which are commercially available or readily obtained using standard techniques.

In some embodiments, the targeting agent is an antibody. The term “antibody” refers to natural or synthetic antibodies that selectively bind a target antigen. The term includes polyclonal and monoclonal antibodies. The antibody can be any type of immunoglobulin that is known in the art. For instance, the antibody can be of any isotype, e.g., IgA, IgD, IgE, IgG, IgM, etc. The antibody can be monoclonal or polyclonal. The antibody can be a naturally-occurring antibody, e.g., an antibody isolated and/or purified from a mammal, e.g., mouse, rabbit, goat, horse, chicken, hamster, human, etc. Alternatively, the antibody can be a genetically-engineered antibody, e.g., a humanized antibody or a chimeric antibody or a fragment, variant, or fusion protein thereof. The antibody can be in monomeric or polymeric form.

In addition to intact immunoglobulin molecules, also included in the term “antibodies” are fragments or polymers or fusions of those immunoglobulin molecules, and human or humanized versions of immunoglobulin molecules that selectively bind the target antigen. Exemplary fragments and fusions include, but are not limited to, single chain antibodies, single chain variable fragments (scFv), di-scFv, tri-scFv, diabody, triabody, teratbody, disulfide-linked Fvs (sdFv), Fab′, F(ab′)₂, Fv, and single domain antibody fragments (sdAb).

In some embodiments, the targeting moiety can be or include one, two, or more scFv. For example, the targeting moiety can be a scFv or a di-scFv.

### 2. Exemplary Methods for Attaching Targeting Agents

Targeting moieties, therapeutic molecules, and other functional moieties can be coupled to particles using standard techniques. For example, the moieties and molecules can be coupled directly or indirectly to the shell or coating.

Functionality refers to conjugation of a ligand to the surface of the particle via a functional chemical group (carboxylic acids, aldehydes, amines, sulfhydryls and hydroxyls) present on the surface of the particle and present on the ligand to be attached. Functionality may be introduced into the particles in at least two ways. The first is during the preparation of the particles, for example during by incorporation of a shell and/or coating with chemical groups.

A second is post-particle preparation, by direct crosslinking particles and ligands with homo- or heterobifunctional crosslinkers. This second procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation.

One useful protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. Typically, the “coupling” of the ligand to the “activated” polymer is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfo NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

By using either of these protocols it is possible to “activate” almost all polymers containing either hydroxyl or carboxyl groups in a suitable solvent system that will not dissolve the polymer matrix (i.e., shell and/or coating)

A useful coupling procedure for attaching ligands with free hydroxyl and carboxyl groups to polymers involves the use of the cross-linking agent, divinylsulfone. This method would be useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine.

Any suitable coupling method known to those skilled in the art for the coupling of ligands and polymers with double bonds, including the use of UV crosslinking, may be used for attachment of molecules to the polymer.

Coupling is preferably by covalent binding but it may also be indirect, for example, through a linker bound to the polymer or through an interaction between two molecules such as strepavidin and biotin. It may also be by electrostatic attraction by dip-coating.

In the experiments below, antibodies were conjugated to coated particles using EDC/NHS chemistry.

### 3. Exemplary Target Molecules

a. Targeting Antigen Presenting Cells

In preferred embodiments, the targeting agent facilitate targeting of the nanoparticle to antigen presenting cells such as dendritic cells. Of the main types of antigen-presenting cells (B cell, macrophages and DCs), the DC is the most potent and is responsible for initiating all antigen-specific immune responses. One biological feature of DCs is their ability to sense conditions under which antigen is encountered, initiating a process of DC maturation. Using receptors for various microbial and inflammatory products, DCs respond to antigen exposure in different ways depending on the nature of the pathogen (virus, bacteria, protozoan) encountered. This information is transmitted to T cells by altered patterns of cytokine release at the time of antigen presentation in lymph nodes, altering the type of T cell response elicited. Thus, targeting DCs provides the opportunity not only to quantitatively enhance the delivery of antigen and antigen responses in general, but to qualitatively control the nature of the immune response depending on the desired vaccination outcome.

Dendritic cells express a number of cell surface receptors that can mediate the endocytosis of bound antigen. Targeting exogenous antigens to internalizing surface molecules on systemically-distributed antigen presenting cells facilitates uptake of antigens and thus overcomes a major rate-limiting step in immunization and thus in vaccination.

Dendritic cell targeting molecules include monoclonal or polyclonal antibodies or fragments thereof that recognize and bind to epitopes displayed on the surface of dendritic cells. Dendritic cell targeting molecules also include ligands which bind to a cell surface receptor on dendritic cells. One such receptor, the lectin DEC-205, has been used in vitro and in mice to boost both humoral (antibody-based) and cellular (CD8 T cell) responses by 2-4 orders of magnitude (Hawiger, et al., J. Exp. Med., 194 (6): 769-79 (2001); Bonifaz, et al., J. Exp. Med., 196 (12): 1627-38 (2002); Bonifaz, et al., J. Exp. Med., 199 (6): 815-24 (2004)). In these experiments, antigens were fused to an anti-DEC205 heavy chain and a recombinant antibody molecule was used for immunization.

A variety of other endocytic receptors, including a mannose-specific lectin (mannose receptor) and IgG Fc receptors, have also been targeted in this way with similar enhancement of antigen presentation efficiency. Other suitable receptors which may be targeted include, but are not limited to, DC-SIGN, 33D1, SIGLEC-H, DCIR, CD11c, heat shock protein receptors and scavenger receptors.

Other receptors which may be targeted include the toll-like receptors (TLRs). TLRs recognize and bind to pathogen-associated molecular patterns (PAMPs). PAMPs target the TLR on the surface of the dendritic cell and signals internally, thereby potentially increasing DC antigen uptake, maturation and T-cell stimulatory capacity. PAMPs conjugated to the particle surface or co-encapsulated include unmethylated CpG DNA (bacterial), double-stranded RNA (viral), lipopolysacharride (bacterial), peptidoglycan (bacterial), lipoarabinomannin (bacterial), zymosan (yeast), mycoplasmal lipoproteins such as MALP-2 (bacterial), flagellin (bacterial) poly(inosinic-cytidylic) acid (bacterial), lipoteichoic acid (bacterial) or imidazoquinolines (synthetic). Thus, in some embodiments, the disclosed nanoparticles are preferably conjugated with targeting moieties to enhance the uptake of the nanoparticle by DCs. In one embodiment, the nanoparticles are conjugated to antibodies which specifically bind to molecules on the surface of DCs. Antigens present on the surface of DCs include, but are not limited to, DEC-205 (CD-205), DC-SIGN, and mannose receptor (MR), Fc receptors, and CD40. For example, an anti-DEC-205 antibody may be conjugated to carbon nanoparticles in order to augment the uptake of the nanoparticle by DCs. Anti CD-205, DC-SIGN, MR and CD40 antibodies are commercially available (Bio-Rad, product code MCA4755 (anti-CD205); R &D systems; catalog #MAB 161 (anti DC-SIGN); Abcam, an8918 (anti MR antibody). CP-870,893 (Pfizer) is a fully human CD40 agonist IgG2 mAb that exhibits immune-mediated and non-immune mediated effects on tumor cell death (Gladue et al. J Clin Oncol. 2006; 24 (18S): 103s). SGN-40 is a humanized IgG1 immunoglobulin and a partial agonist of CD40 that induces apoptosis and antibody-dependent cellular cytotoxicity against a panel of malignant B cell lines in vitro and results in tumor regression in human multiple myeloma and lymphoma xenograft models in vivo (Tai, et al., Cancer Res. 2004; 64(8):2846-52; Law, et al., Cancer Res. 2005; 65(18):8331-8. And Kelley, et al., Br J Pharmacol. 2006; 148(8):1116-23). In another embodiment, nanoparticles may be conjugated to receptor ligands, wherein the corresponding receptor is expressed on the surface of the DCs. For example, a DC receptor may include, but is not limited to, ICAM-2 and PDI.

In some of the examples below, the targeting agent is an anti-CD205 that targets dendritic cells.

b. Targeting T Cells and Other Immune Cells

In some embodiments, the targeting moiety targets T cells. The T cells can be effector cells (e.g., cytotoxic, helper, regulatory, or a combination thereof), memory T cells, Gamma-delta T cells (γδ T cells), Follicular helper T cells (Tfh), Natural killer T cells (NKT cells), or a combination thereof.

Targets include, but are not limited to, CD3, CD4, CD8, CD103, C-X-C motif chemokine receptor 6 (CXCR6), CD69, PD-1, CD90, TIGIT, CCR7, CD45RA, CD45RO, CD62L, CD95, 4-1BB, LAG-3, TIM-3, and CTLA4.

In particular embodiments, the target T cells are or include CD+8 T cells.

Exemplary T cell targeting moieties are discussed in US published application No. 20210386782.

Exemplary antibodies are discussed in more detail below. It will be appreciated that not only can the antibodies themselves be used in the disclosed compositions and methods, but that the complementarity determining regions (CDRs), preferable in heavy and light chain variable region frameworks, and in some examples the entire heavy and light chain variable regions, can be used to form other antibody formats discussed herein including but not limited to humanized and/or chimeric antibodies, fusion proteins such as scFv, etc. Thus, such antibodies and antibodies fragments including the CDRs, preferably in their native orientation, preferably in a suitable heavy and light chain variable region, are expressly provided for each of the exemplified antibodies herein.

### CD3

CD3 is expressed by all T cells. GenBank accession numbers for exemplary sequences for human CD3 proteins include, for example, T-cell surface glycoprotein CD3 delta chain isoform A P_000723.1 precursor; T-cell surface glycoprotein CD3 delta chain isoform B NP_001035741.1; precursor T-cell surface glycoprotein CD3 epsilon chain P07766.2 precursor; T-cell surface glycoprotein CD3 gamma chain NP_000064.1 precursor; T-cell surface glycoprotein CD3 zeta chain isoform 1 NP_932170.1 precursor and T-cell surface glycoprotein CD3 zeta chain isoform 2 NP_000725.1 precursor.

Exemplary Anti-CD3 antibodies include, but are not limited to, those disclosed in US20150166661, US20170204194, U.S. Pat. Nos. 7,728,114, 8,551,478, US20140193399, US20030216551, US20060275292, WO2017136659, and WO2016179003. Anti-CD3 antibodies specific for human CD3 available from commercial suppliers, include, but are limited to, clone 289-13801 (Cat. No. ABIN234581, Antibodies-Online), clone 4AOKT3 (Cat. No. ABIN2145039, Antibodies-Online), clone 4D10A6 (Cat. No. ABIN969472), clone B477 (Cat. No. ABIN965782, Antibodies-Online), clone B-B11 (Cat. No. ABIN1383795, Antibodies-Online), clone hCD3 (Cat. No. ABIN2136389, Antibodies-Online), clone HIT3a (Cat. No. ABIN2136387, Antibodies-Online), clone Okt 03 (Cat. No. ABIN457398, Antibodies-Online), clone UCHT1 (Cat. No. ABIN135720, Antibodies-Online), clone BC3 (Cat. No. 830301, BioLegend), clone Hu113 (Cat. No. MAB9929-100, R&D Systems Inc.), clone B-B11 (Cat. No. AM31215PU-N, Origene), clone N26-R (Cat. No. NBP1-79054, Novus Biologicals Canada), clone 1A7E5G5 (Cat. No. 10977-MM03, Sino Biological Inc), clone UCHT-1 (Cat. No. T-1363, BMA Biomedicals).

### CD4

CD4 is expressed by helper T cells. Targeting this antigen could be used to selectively deplete CD4 T cells in diseases where CD4 T cells preferentially contribute to pathology. For example, malignant T cells in cutaneous T cell lymphoma are usually CD4+ and targeting these cells could be used to selectively deplete malignant T cells from skin without harming the CD8+ T cell population.

A sequence for human CD4 protein is available in GenBank at Acc. No. NP_000607.1. Anti-CD4 antibodies include, but are not limited to, those disclosed in U.S. Pat. Nos. 7,452,534, 5,871,732, 8,877,913, 8,399,621, 7,947,272, 7,452,981, 8,440,806, 8,586,715, 8,673,304, and 8,685,651. Anti-CD4 antibodies specific for human CD4 available from commercial suppliers, include, but are not limited to, clone 8 (Cat. No. 10400-MM08, Sino Biological Inc.), clone 22 (Cat. No. 10400-MM22, Sino Biological Inc.), clone 6F7B4C5 (Cat. No. 10400-MM03, Sino Biological Inc.), clone CE9.1 (Cat. No. A1091-200, Biovision Inc.), clone CL0395 (Cat. No. AMAb90754, Atlas Antibodies), clone 34915 (Cat. No. MAB3791, R&D Systems), clone 34930 (Cat. No. MAB379-100, R&D Systems), clone 10B5 (Cat. No. GTX84720, GeneTex), clone 13B8.2 (Cat. No. GTX44212, GeneTex), clone MEM-241 (Cat. No. GTX21089, GeneTex), clone 4A11 (Cat. No. ABIN2136522, Antibodies Online), clone 4B12 (Cat. No. ABIN180655, Antibodies Online), and clone 6 Eli) (Cat No. ABIN2136524, Antibodies Online).

### CD8

CD8 is expressed by cytotoxic T cells. In some inflammatory diseases, such as allograft rejection, CD8+ T cells are thought to cause the majority of tissue damage (Harper, S. J. et al., (2015). Proc Natl Acad Sci USA 112(41):12788-12793). Thus, depending on the biology of the inflammatory process, it may be desirable to deplete CD8+ T cells without harming other T cell subsets. The sequence for human CD8 protein is available in GenBank at Acc. No. NP_001759.3. Anti-CD8 antibodies include, but are not limited to, those disclosed in U.S. Pat. No. 9,518,131, WO9015152, and US20090304659. Anti-CD8 antibodies specific for human CD8 available from commercial suppliers, include, but are not limited to clone C8/144B (Cat. No. 925-MSM2-P1, Enquire Bioreagents), clone C8/468 (Cat. No. 925-MSM1-P1, Enquire Bioreagents), clone 37006 (Cat. No. MAB1509, R&D Systems), clone 2ST8.5H7 (Cat. No. GTX75282, GeneTex), clone LT8 (Cat. No. LT8, GeneTex), clone OKT-8 (Cat. No. GTX14198, GeneTex), clone Bu88 (Cat. No. AM05583PU-N, Origene Technologies), clone B-Z31 (Cat. No. AM31251PU-N, Origene Technologies), clone MCD8 (Cat. No. AM39011PU-N, Origene Technologies), clone RAVB3 (Cat. No. AM06078PU-N, Origene Technologies), clone RFT-8 (Cat. No. AM08158PU-N, Origene Technologies), clone 14 (Cat. No. NBP2-50467, Novus Biologicals Canada), clone X107 (Cat. No. NBP2-50469, Novus Biologicals Canada), and clone UCH-T4 (Cat. No. NBP2-50468, Novus Biologicals Canada).

### CD103

CD103 is expressed by Resident memory T cells (TRM) in peripheral tissues in both humans and mice and is enriched on TRM that populate mucosae and epithelia (Sathaliyawala, T., et al., (2013). Immunity 38 (1): 187-197). CD103 is also known as integrin subunit alpha E (ITGAE). The sequence for human CD103 protein is available in GenBank at Acc. No. NP_002199.3. Anti-CD103 antibodies include, but are limited to, those disclosed in US20110142861, US20110142860, and US20050266001. Anti-CD103 antibodies specific for human CD103 available from commercial suppliers, include, but are limited to, clone B-Ly7 (Cat. No. NBP1-43370H, Novus Biologicals Canada), clone BP6 (Cat. No. NBP2-50446H, Novus Biologicals Canada), clone LF61 (Cat. No. NB100-65272H, Novus Biologicals Canada), clone AX.14 (Cat. No. AM05205PU-N, Origene Technologies), clone B-ly7 (Cat. No. AM39027PU-N, Origene Technologies), clone 3H1798 (Cat. No. C2445-63A, United States Biological), clone 3H1797 (Cat. No. C2445-63, United States Biological), clone 3H1797 (Cat. No. C2445-63J1, United States Biological), and clone 3H1797 (Cat. No. C2445-63K, United States Biological).

### CXCR6

CXCR6 is expressed by TRM in tissues and is required for their optimal development (Zaid, A., (2017). J Immunol 199 (7): 2451-2459). The sequence for human CXCR6 protein is available in GenBank at Acc. No. NP_006555.1. Anti-CXCR6 antibodies include, but are limited to, those disclosed in U.S. Pat. No. 9,872,905 and WO2004019046. Anti-CXCR6 antibodies specific for human CXCR6 available from commercial suppliers, include, but are limited to, clone 56811 (Cat. No. MAB699-100, R&D Systems), clone MM0226-2B44 (Cat. No. NBP2-12243, R&D Systems), clone 14L333 (Cat. No. 216429, R&D Systems), clone K041E5 (Cat. No. 356001, BioLegend), clone K041E5 (Cat. No. 356002, BioLegend), and select polyclonal antibodies specific for human CXCR6 (e.g., Cat. No. GTX77935, GeneTex; Cat. No. SP1286P, Origene Technologies; Cat. No. NLS1102, Novus Biologicals Canada; Cat. No. abx148716, Abbexa; Cat. No. 170358, United States Biological).

### CD69

CD69 is a surface molecule that is expressed at high and constant levels by TRM regardless of activation status in all tissues tested so far, and is the most inclusive marker of TRM in human skin (Watanabe, R. et al. (2015). Science Translational Medicine 7 (279): 279ra239). CD69 is also expressed by activated T cells in tissues, e.g., at inflamed sites, and is upregulated in vitro within 12 hours of stimulation. CD69 is not expressed by circulating T cells or FOXP3 regulatory T cells, at least in human skin (Clark, R. A., et al. (2007). Blood 109(1): 194-202).

The sequence for human CD69 protein is available in GenBank at Acc. No. NP_001772.1. Anti-CD69 antibodies known in the art and useful in the present methods include, but are not limited to, those disclosed in US20150118237, U.S. Pat. No. 8,440,195, US20130224111, U.S. Pat. Nos. 7,867,475, 8,182,816, WO2018074610, and WO2018150066. Anti-CD69 antibodies specific for human CD19 are available from commercial suppliers, include, but are limited to, clone 4AF50 (Cat. No. ABIN2145225, Antibodies-Online), clone FN50 (Cat. No. ABIN302090, Antibodies-Online), clone 298633 (Cat. No. MAB2359-SP, R&D Systems), clone 298614 (Cat. No. MAB23591, R&D Systems), monoclonal anti-CD69 antibody (Cat. No. AM03132PU-N, OriGene TEchnologies), clone 15B5G2 (Cat. No. NBP2-25242SS, Novus Biologicals Canada), clone 7H192 (Cat. No. C2424-01E, US Biological Life Sciences), clone 4H3 (Cat. No. 124672, US Biological Life Sciences), clone 7H192 (Cat. No. C2424-01, US Biological Life Sciences), clone HP-4B3 (Cat. No. LS-C134543-100, LifeSpan BioSciences), or select polyclonal antibodies specific for human CD69 (e.g., Cat. No. ABIN2136942, Antibodies-Online; Cat. No. AF2359, R&D Systems; Cat. No. GTX37447, GeneTex Inc.; Cat. No. AP21168PU-N, OriGene Technologies; Cat. No. 124671, US Biological Life Sciences).

### PD-1 and CTLA4

PD-1 and CTLA4 are proteins found on T cells that helps keep the body's immune responses in check. When PD-1 is bound to another protein called PD-L1, it helps keep T cells from killing other cells, including cancer cells. Similarly, when CTLA-4 is bound to another protein called B7, it helps keep T cells from killing other cells. Some anticancer drugs, called immune checkpoint inhibitors, are used to block PD-1 and CTLA4. When these proteins are blocked, the “brakes” on the immune system are released and the ability of T cells to kill cancer cells is increased.

Anti-PD-1 and anti-CTLA antibodies are known in the art are discussed in more detail elsewhere herein. Any of such antibodies can be used as activity agents and/or targeting moieties.

### CD90

Thy-1 or CD90 (Cluster of Differentiation 90) is a 25-37 kDa heavily N-glycosylated, glycophosphatidylinositol (GPI) anchored conserved cell surface protein with a single V-like immunoglobulin domain, originally discovered as a thymocyte antigen. Antibodies to human CD90 are known in the art, see, for example, F15-42-1 (e.g., ThermoFisher Cat #MA5-16671), eBio5E10 (5E10) (e.g., ThermoFisher Cat #11-0909-42), 2V9S6 (ThermoFisher Cat #MA5-42657), SU35-07 (e.g., ThermoFisher Catalog #MA5-32124), HL1766 (ThermoFisher Catalog #MA5-47174), etc.

### TIGIT

TIGIT is expressed by activated CD8+ T and CD4+ T cells, natural killer (NK) cells, regulatory T cells (Tregs), and follicular T helper cells in humans. In sharp contrast with DNAM-1/CD226, TIGIT is weakly expressed by naive T cells. Antibodies to human TIGIT are known in the art, see, for example, MBSA43 (e.g., ThermoFisher Cat #12-9500-42), BLR047F (e.g., ThermoFisher Cat #A700-047), OTI3B6 (e.g., ThermoFisher Cat #CF812550), OTI5G1 (e.g., ThermoFisher Cat #CF812567), OTI3A10 (e.g., ThermoFisher Cat #CF813029), etc.

### CD45RA and CD45RO

The tyrosine phosphatase CD45 is alternatively spliced to generate isoforms of different molecular weights (180-220 kDa) which are differentially expressed on hematopoietic cells (LaSalle and Haflter, et al., Cell Immunol. 1991 November; 138 (1): 197-206. doi: 10.1016/0008-8749 (91) 90144-z.). Monoclonal antibodies reacting with either the 180-kDa (UCHL-1, CD45RO) or the 200- to 220-kDa (2H4, CD45RA) isoform have been used to subdivide T cell populations based on their expression of one or the other of these two epitopes. CD45RA T cells have “naive” characteristics of unresponsiveness to recall antigens and prominence in cord blood, while CD45RO T cells are considered “memory” T cells because they proliferate to recall antigens and increase following PHA activation of cord blood.

Antibodies to human CD45RA are known in the art, see, for example, HI100 (e.g., ThermoFisher Cat #11-0458-42), MEM-56 (e.g., ThermoFisher Cat #MHCD45RA01), 4KB5 (e.g., ThermoFisher Cat #MA5-12490), JS-83 (e.g., ThermoFisher Cat #11-9979-42), etc.

Antibodies to human CD45RO are known in the art, see, for example, UCHL1 (e.g., ThermoFisher Cat #MA5-11532), IL-A116 (e.g., ThermoFisher Cat #MA5-28402), T200, 797 (e.g., ThermoFisher Cat #5788-MSM7-P1), etc.

### CD62L

L-selectin, also known as CD62L, is a cell adhesion molecule found on the cell surface of leukocytes, and the blastocyst. L-selectin is expressed on naive T cells and is rapidly shed following T cell priming. L-selectin expression is re-activated in cytotoxic T cells once they exit the lymph node. Mature central memory T cells express L-selectin while effector memory cells do not. L-selectin is also expressed by naive B cells, with the loss of L-selectin distinguishing activated B cells destined to differentiate to antibody-secreting cells. L-selectin is expressed on circulating neutrophils and is shed following neutrophil priming. Expression of L-selectin in neutrophils decreases with neutrophil aging. Classical monocytes express high levels of L-selectin while in circulation. Shedding of L-selectin from monocytes occurs during trans-endothelial migration.

Antibodies to human CD62L are known in the art, see, for example, LT-TD180 (e.g., ThermoFisher Cat #MA1-19715), DREG56 (e.g., ThermoFisher Cat #17-0629-42), IVA94 (e.g., ThermoFisher Cat #MA5-44129), etc.

### CD95

The Fas receptor, also known as Fas, FasR, apoptosis antigen 1 (APO-1 or APT), cluster of differentiation 95 (CD95) or tumor necrosis factor receptor superfamily member 6 (TNFRSF6), is a protein that in humans is encoded by the FAS gene. CD95 (Fas/APO-1) and its ligand, CD95L, have long been viewed as a death receptor/death ligand system that mediates apoptosis induction to maintain immune homeostasis. In addition, these molecules are important in the immune elimination of virus-infected cells and cancer cells.

Antibodies to human CD95 are known in the art, see, for example, JJ0942 (e.g., ThermoFisher Cat #MA5-32489), DX2 (e.g., ThermoFisher Cat #11-0959-42), H.831.6 (e.g., ThermoFisher Cat #MA5-14882), SM1/23 (e.g., ThermoFisher Cat #17-0959-42), etc.

### 4-1BB

4-1BB (CD137; TNFRS9), an activation-induced costimulatory molecule, is an important regulator of immune responses. 4-1BB was originally discovered from activated cells, and on account of this, it was originally referred to as induced lymphocyte activation (ILA) in humans, but is also constitutively expressed on a number of cells, albeit at low levels, including Foxp3+ Tregs and DCs (Vinay and Kwon BMB Rep. 2014 March; 47 (3): 122-129).

Antibodies to human 4-1BB are known in the art, see, for example, 4B4 (e.g., ThermoFisher Cat #11-1379-42), ARC1963 (e.g., ThermoFisher Cat #MA5-38063), BBK-2 (e.g., ThermoFisher Cat #MA5-13739), 4H3 (e.g., ThermoFisher Cat #25-5906-42), 2G1 (e.g., ThermoFisher Cat #MA5-42580), 819 (e.g., ThermoFisher Cat #MA5-46628), etc.

### LAG-3

LAG-3 (CD223) is a cell surface molecule expressed on activated T cells (Huard et al. Immunogenetics 39:213-217, 1994), NK cells (Triebel et al. J Exp Med 171:1393-1405, 1990), B cells (Kisielow et al. Eur J Immunol 35:2081-2088, 2005), and plasmacytoid dendritic cells (Workman et al. J Immunol 182:1885-1891, 2009) that plays an important role in the function of these lymphocyte subsets. In addition, the interaction between LAG-3 and its major ligand, Class II MHC, is thought to play a role in modulating dendritic cell function (Andreae et al. J Immunol 168:3874-3880, 2002), and recent preclinical studies have documented a role for LAG-3 in CD8 T cell exhaustion (Blackburn et al. Nat Immunol 10:29-37, 2009).

Antibodies to human LAG-3 are known in the art, see, for example, 3DS223H (e.g., ThermoFisher Cat #17-2239-42), BLR028F (e.g., ThermoFisher Cat #A700-028), 1F14 (e.g., ThermoFisher Cat #80867-1-RR100UL), OTI8F6 (e.g., ThermoFisher Cat #A700-027), etc.

### TIM-3

Tim-3 is a co-inhibitory receptor that is expressed on IFN-γ-producing T cells, FoxP3+ Treg cells and innate immune cells (macrophages and dendritic cells) where it has been shown to suppress their responses upon interaction with their ligand(s) (Das, et al., Immunol Rev. 2017 March; 276(1): 97-111).

Antibodies to human TIM-3 are known in the art, see, for example, F38-2E2 (e.g., ThermoFisher Cat #78-3109-42), 4C4G3 (e.g., ThermoFisher Cat #60355-1-IG), 1E5 (e.g., ThermoFisher Cat #MA5-32841), 1E6 (e.g., ThermoFisher Cat #MA5-32839), 1E3 (e.g., ThermoFisher Cat #368-3109-42), etc.

### E. Active Agents

The disclosed particles can have a molecular and even therapeutic effect without any additional active agent, and thus in some embodiments, the particles alone are the active material and the particles do not include (i.e., are free from) an additional active agent. Alternatively, the particle can optionally include one or more active agents. For example, in some embodiments, the outer layer or coating is, or includes an active agent. In some embodiments, the active agent or agents are conjugated to a component of the hydrophilic layer or otherwise attached to the surface of the layer, or incorporated, loaded or encapsulated into the layer itself. In some such embodiments, the core of the particles remains free of additional active agents. Additionally or alternatively, active agents, including, but not limited to those discussed here, can be separate from the particles and administered in a different formulation (i.e., different admixture), or the same formulation (i.e., same admixture). Thus, contemplated are particles with or without active agents, pharmaceutical compositions include particles with or without active agents alone or in further combination with active agents, and methods that include administering pharmaceutical compositions to subject in need thereof alone or in combination with (together or separately) one or more active agents are other adjunct treatments. Any of the active agents provided in this section or elsewhere herein can serve any one or more of these roles.

The active agent or agents can be, for example, nucleic acids, proteins, and/or small molecules. Exemplary active agents include, for example, tumor antigens, CD4+ T-cell epitopes, cytokines, chemotherapeutic agents, radionuclides, small molecule signal transduction inhibitors, photothermal antennas, immunologic danger signaling molecules, other immunotherapeutics, enzymes, antibiotics, antivirals, anti-parasites (helminths, protozoans), growth factors, growth inhibitors, hormones, hormone antagonists, antibodies and bioactive fragments thereof (including humanized, single chain, and chimeric antibodies), antigen and vaccine formulations (including adjuvants), peptide drugs, anti-inflammatories immunomodulators (including ligands that bind to Toll-Like Receptors (including but not limited to CpG oligonucleotides) to activate the innate immune system, molecules that mobilize and optimize the adaptive immune system, molecules that activate or up-regulate the action of cytotoxic T lymphocytes, natural killer cells and helper T-cells, and molecules that deactivate or down-regulate suppressor or regulatory T-cells), agents that promote uptake of the delivery vehicle into cells (including dendritic cells and other antigen-presenting cells), nutraceuticals such as vitamins, and oligonucleotide drugs (including DNA, RNAs, antisense, aptamers, small interfering RNAs, ribozymes, external guide sequences for ribonuclease P, and triplex forming agents).

### 1. Antigens

Antigens may be provided as single antigens or may be provided in combination and can be derived from tumors, infectious agents, or elsewhere. These may be particularly preferred additional agents when antigen presenting cells are targeted.

a. Tumor Antigens

A tumor antigen can be a tumor specific antigen (present only on tumor cells) or a tumor associated antigen (present on some tumor cells and also in some normal cells).

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17 (4B): 2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include or be a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Acc. No. S73003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including but not limited to MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARa fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15 (58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion proteins.

Other examples of cancer associated antigens include, but are not limited to mesothelin, EGFRvIII, TSHR, CD19, CD123, CD22, CD30, CD171, CS-1, CLL-1, CD33, GD2, GD3, BCMA, Tn Ag, prostate specific membrane antigen (PSMA), ROR1, FLT3, FAP, TAG72, CD38, CD44v6, CEA, EPCAM, B7H3, KIT, IL-13Ra2, interleukin-11 receptor a (IL-11Ra), PSCA, PRSS21, VEGFR2, LewisY, CD24, platelet-derived growth factor receptor-beta (PDGFR-beta), SSEA-4, CD20, Folate receptor alpha (FRa), ERBB2 (Her2/neu), MUC1, epidermal growth factor receptor (EGFR), NCAM, Prostase, PAP, ELF2M, Ephrin B2, IGF-1 receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In other embodiments, the antigen is one that is expressed by neovasculature associated with a tumor. The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed fusion proteins.

Suitable antigens are known in the art and are available from commercial government and scientific sources. The antigens may be purified or partially purified polypeptides derived from tumors or viral or bacterial sources. The antigens can be recombinant polypeptides produced by expressing DNA encoding the polypeptide antigen in a heterologous expression system.

b. Viral Antigens

A viral antigen can be isolated from and or derived from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue DINS1, Dengue DINS2, and Dengue DINS3.

Viral antigens may be derived from a particular strain such as a papilloma virus, a herpes virus, e.g., herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

c. Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

d. Parasite Antigens

Parasite antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

### 2. Chemotherapeutic Drugs

Exemplary active agents include, for example, chemotherapeutics, especially antineoplastic drugs. The majority of chemotherapeutic drugs can be divided into alkylating agents, antimetabolites, anthracyclines, plant alkaloids, topoisomerase inhibitors, monoclonal antibodies, and other antitumor agents. In particular embodiments, the additional active agent is an alkylating agent (such as temozolomide, cisplatin, carboplatin, oxaliplatin, mechlorethamine, cyclophosphamide, chlorambucil, dacarbazine, lomustine, carmustine, procarbazine, chlorambucil and ifosfamide), an antimetabolite (such as fluorouracil, gemcitabine, methotrexate, cytosine arabinoside, fludarabine, and floxuridine), anantimitotic or vinca alkaloid (such as vincristine, vinblastine, vinorelbine, and vindesine), an anthracycline (including doxorubicin, daunorubicin, valrubicin, idarubicin, and epirubicin, as well as actinomycins such as actinomycin D), a cytotoxic antibiotic (including mitomycin, plicamycin, and bleomycin), or a topoisomerase inhibitor (including camptothecins such as irinotecan and topotecan and derivatives of epipodophyllotoxins such as amsacrine, etoposide, etoposide phosphate, and teniposide).

### 3. Immune System Modulators

Immune system modulators are a type of immunotherapy that enhance the body's immune response against cancer. Immune system modulators include cytokines, Bacillus Calmette-Guerin (BCG), and immunomodulatory drugs. Cytokines that are sometimes used to treat cancer include Interferons (INFs) and Interleukins. Researchers have found that one type of interferon, called INF-alpha, can enhance an immune response to cancer cells by causing certain white blood cells, such as natural killer cells and dendritic cells, to become active. INF-alpha may also slow the growth of cancer cells or promote their death. IL-2 boosts the number of white blood cells in the body, including killer T cells and natural killer cells. Increasing these cells can cause an immune response against cancer. IL-2 also helps B cells (another type of white blood cell) produce certain substances that can target cancer cells. BCG is used to treat bladder cancer. When inserted directly into the bladder with a catheter, BCG causes an immune response against cancer cells. Immunomodulatory drugs (also called biological response modifiers) stimulate the immune system. They include thalidomide (Thalomid®); lenalidomide (Revlimid®); pomalidomide (Pomalyst®); and imiquimod (Aldara®, Zyclara®).

### 4. Immune Checkpoint Modulators

The active agents can be immune checkpoint modulators. Immune checkpoints can be stimulatory or inhibitory, and tumors can use these checkpoints to protect themselves from immune system attacks. Currently approved checkpoint therapies block inhibitory checkpoint receptors, but investigations into therapies that activate stimulatory checkpoints are also underway. Thus, the immune checkpoint modulator can be one that blocks an inhibitory checkpoint, or activates a stimulatory checkpoint. Typically, the immune checkpoint modulator is one that induces or otherwise activates or increases an immune response against target cells for example cancer cells or infected cells.

In preferred embodiments, the immune checkpoint modulator blocks an inhibitory checkpoint. Blockade of negative feedback signaling to immune cells thus results in an enhanced immune response against tumors. Thus, in some embodiments the immune checkpoint modulator is administered to the subject in an effective amount to block an inhibitory checkpoint. Exemplary compounds are those that block or otherwise inhibit, for example, PD-1, PD-L1, or CTLA4.

a. PD-1 Antagonists

In some embodiments, the active agents are PD-1 antagonists. Activation of T cells normally depends on an antigen-specific signal following contact of the T cell receptor (TCR) with an antigenic peptide presented via the major histocompatibility complex (MHC) while the extent of this reaction is controlled by positive and negative antigen-independent signals emanating from a variety of co-stimulatory molecules. The latter are commonly members of the CD28/B7 family. Conversely, Programmed Death-1 (PD-1) is a member of the CD28 family of receptors that delivers a negative immune response when induced on T cells. Contact between PD-1 and one of its ligands (B7-H1 or B7-DC) induces an inhibitory response that decreases T cell multiplication and/or the strength and/or duration of a T cell response. Suitable PD-1 antagonists are described in U.S. Pat. Nos. 8,114,845, 8,609,089, and 8,709,416, and include compounds or agents that either bind to and block a ligand of PD-1 to interfere with or inhibit the binding of the ligand to the PD-1 receptor, or bind directly to and block the PD-1 receptor without inducing inhibitory signal transduction through the PD-1 receptor.

In some embodiments, the PD-1 receptor antagonist binds directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

It is believed that PD-1 signaling is driven by binding to a PD-1 ligand (such as B7-H1 or B7-DC) in close proximity to a peptide antigen presented by major histocompatibility complex (MHC) (see, for example, Freeman, Proc. Natl. Acad. Sci. U. S. A, 105:10275-10276 (2008)). Therefore, proteins, antibodies or small molecules that prevent co-ligation of PD-1 and TCR on the T cell membrane are also useful PD-1 antagonists.

In preferred embodiments, the PD-1 receptor antagonists are small molecule antagonists or antibodies that reduce or interfere with PD-1 receptor signal transduction by binding to ligands of PD-1 or to PD-1 itself, especially where co-ligation of PD-1 with TCR does not follow such binding, thereby not triggering inhibitory signal transduction through the PD-1 receptor.

Other PD-1 antagonists include antibodies that bind to PD-1 or ligands of PD-1 such as PD-L1 (also known as B7-H1) and PD-L2 (also known as B7-DC), and other antibodies.

Suitable anti-PD-1 antibodies include, but are not limited to, those described in the following publications:
PCT/IL03/00425 (Hardy et al., WO/2003/099196)
PCT/JP2006/309606 (Korman et al., WO/2006/121168)
PCT/US2008/008925 (Li et al., WO/2009/014708)
PCT/JP03/08420 (Honjo et al., WO/2004/004771)
PCT/JP04/00549 (Honjo et al., WO/2004/072286)
PCT/IB2003/006304 (Collins et al., WO/2004/056875)
PCT/US2007/088851 (Ahmed et al., WO/2008/083174)
PCT/US2006/026046 (Korman et al., WO/2007/005874)
PCT/US2008/084923 (Terrett et al., WO/2009/073533)
Berger et al., Clin. Cancer Res., 14:30443051 (2008).

A specific example of an anti-PD-1 antibody is MDX-1106 (see Kosak, US20070166281 (pub. 19 Jul. 2007) at par. 42), a human anti-PD-1 antibody, preferably administered at a dose of 3 mg/kg.

Exemplary anti-B7-H1 antibodies include, but are not limited to, those described in the following publications:
PCT/US06/022423 (WO/2006/133396, pub. 14 Dec. 2006)
PCT/US07/088851 (WO/2008/083174, pub. 10 Jul. 2008)
US 2006/0110383 (pub. 25 May 2006)

A specific example of an anti-B7-H1 antibody is MDX-1105 (WO/2007/005874, published 11 Jan. 2007)), a human anti-B7-H1 antibody.

For anti-B7-DC antibodies see U.S. Pat. Nos. 7,411,051, 7,052,694, 7,390,888, and U.S. Published Application No. 2006/0099203.

The antibody can be a bi-specific antibody that includes an antibody that binds to the PD-1 receptor bridged to an antibody that binds to a ligand of PD-1, such as B7-H1. In some embodiments, the PD-1 binding portion reduces or inhibits signal transduction through the PD-1 receptor.

Other exemplary PD-1 receptor antagonists include, but are not limited to B7-DC polypeptides, including homologs and variants of these, as well as active fragments of any of the foregoing, and fusion proteins that incorporate any of these. In a preferred embodiment, the fusion protein includes the soluble portion of B7-DC coupled to the Fc portion of an antibody, such as human IgG, and does not incorporate all or part of the transmembrane portion of human B7-DC.

The PD-1 antagonist can also be a fragment of a mammalian B7-H1, preferably from mouse or primate, preferably human, wherein the fragment binds to and blocks PD-1 but does not result in inhibitory signal transduction through PD-1. The fragments can also be part of a fusion protein, for example an Ig fusion protein.

Other useful polypeptides PD-1 antagonists include those that bind to the ligands of the PD-1 receptor. These include the PD-1 receptor protein, or soluble fragments thereof, which can bind to the PD-1 ligands, such as B7-H1 or B7-DC, and prevent binding to the endogenous PD-1 receptor, thereby preventing inhibitory signal transduction. B7-H1 has also been shown to bind the protein B7.1 (Butte et al., Immunity, Vol. 27, pp. 111-122, (2007)). Such fragments also include the soluble ECD portion of the PD-1 protein that includes mutations, such as the A99L mutation, that increases binding to the natural ligands (Molnar et al., PNAS, 105:10483-10488 (2008)). B7-1 or soluble fragments thereof, which can bind to the B7-H1 ligand and prevent binding to the endogenous PD-1 receptor, thereby preventing inhibitory signal transduction, are also useful.

PD-1 and B7-H1 anti-sense nucleic acids, both DNA and RNA, as well as siRNA molecules can also be PD-1 antagonists. Such anti-sense molecules prevent expression of PD-1 on T cells as well as production of T cell ligands, such as B7-H1, PD-L1 and/or PD-L2. For example, siRNA (for example, of about 21 nucleotides in length, which is specific for the gene encoding PD-1, or encoding a PD-1 ligand, and which oligonucleotides can be readily purchased commercially) complexed with carriers, such as polyethyleneimine (see Cubillos-Ruiz et al., J. Clin. Invest. 119 (8): 2231-2244 (2009), are readily taken up by cells that express PD-1 as well as ligands of PD-1 and reduce expression of these receptors and ligands to achieve a decrease in inhibitory signal transduction in T cells, thereby activating T cells.

Exemplary PD-1 inhibitors include, but are not limited to,
Pembrolizumab (formerly MK-3475 or lambrolizumab, Keytruda) was developed by Merck and first approved by the Food and Drug Administration in 2014 for the treatment of melanoma.
Nivolumab (Opdivo) was developed by Bristol-Myers Squibb and first approved by the FDA in 2014 for the treatment of melanoma.
pidilizumab, by CureTech
AMP-224, by GlaxoSmithKline and MedImmune
AMP-514, by GlaxoSmithKline and MedImmune
PDR001, by Novartis
cemiplimab, by Regeneron and Sanofi

Exemplary PD-L1 inhibitors include, but are not limited to,
Atezolizumab (Tecentriq) is a fully humanised IgG1 (immunoglobulin 1 antibody developed by Roche Genentech. In 2016, the FDA approved atezolizumab for urothelial carcinoma and non-small cell lung cancer.
Avelumab (Bavencio) is a fully human IgG1 antibody developed by Merck Serono and Pfizer. Avelumab is FDA approved for the treatment of metastatic merkel-cell carcinoma. It failed phase III clinical trials for gastric cancer.
Durvalumab (Imfinzi) is a fully human IgG1 antibody developed by AstraZeneca. Durvalumab is FDA approved for the treatment of urothelial carcinoma and unresectable non-small cell lung cancer after chemoradiation.
BMS-936559, by Bristol-Myers Squibb
CK-301, by Checkpoint Therapeutics

See, e.g., Iwai, et al., Journal of Biomedical Science, (2017) 24:26, DOI 10.1186/s12929-017-0329-9.

b. CTLA4 Antagonists

Other molecules useful in mediating the effects of T cells in an immune response are also contemplated as active agents. For example, in some embodiments, the molecule is an agent binds to an immune response mediating molecule that is not PD-1. In a preferred embodiment, the molecule is an antagonist of CTLA4, for example an antagonistic anti-CTLA4 antibody. An example of an anti-CTLA4 antibody is described in PCT/US2006/043690 (Fischkoff et al., WO/2007/056539).

Dosages for anti-PD-1, anti-B7-H1, and anti-CTLA4 antibody, are known in the art and can be in the range of 0.1 to 100 mg/kg, with shorter ranges of 1 to 50 mg/kg preferred and ranges of 10 to 20 mg/kg being more preferred. An appropriate dose for a human subject is between 5 and 15 mg/kg, with 10 mg/kg of antibody (for example, human anti-PD-1 antibody, like MDX-1106) most preferred.

Specific examples of CTLA antagonists include Ipilimumab, also known as MDX-010 or MDX-101, a human anti-CTLA4 antibody, preferably administered at a dose of about 10 mg/kg, and Tremelimumab a human anti-CTLA4 antibody, preferably administered at a dose of about 15 mg/kg. See also Sammartino, et al., Clinical Kidney Journal, 3 (2): 135-137 (2010), published online December 2009.

In other embodiments, the antagonist is a small molecule. A series of small organic compounds have been shown to bind to the B7-1 ligand to prevent binding to CTLA4 (see Erbe et al., J. Biol. Chem., 277:7363-7368 (2002). Such small organics could be administered alone or together with an anti-CTLA4 antibody to reduce inhibitory signal transduction of T cells.

### 5. Immune Cell Modulators

The active agents can be immune cell modulators. Immune cell modulators include, but are not limited to, compounds that increase survival, expansion, activity, and/or persistence of T cells. Such compounds include inhibitors of the PI3K/A T/mTOR pathway including, but not limited to, BEZ235, LY294002, GDC-0941, BYL719, GSK2636771, TGX-221, AS25242, CAL-101, IPI-145, MK-2206, GSK690693, GDC-0068, A-674563, CCT128930, AZD8055, INK128, rapamycin, PF-04691502, everolimus, BI-D1870, H89, PF-4708671, FMK, AT7867, NU7441, PI-103, NU7026, PIK-75, ZSTK474, and PP-121. See, e.g., WO 2015/188119.

Protein Kinase C (PKC) antagonists can further enhance a calcium-based to boost T-cell immunity. Examples include, but are not limited to, phorbol 12-myristate 13-acetate (PMA) (also known as 12-O-tetradecanoylphorbol 13-acetate (TPA), Ingenol 3-angelate (I3A), bryostatin, bisindolylmaleimide I (otherwise known as 2-[1-(3-Dimethylaminopropyl) indol-3-yl]-3-(indol-3-yl) maleimide or GFX (GF109203X)), Calphostin C, and Go6976 (5,6,7,13-Tetrahydro-13-methyl-5-oxo-12H-indolo[2,3-a]pyrrolo[3,4-c]carbazole-12-propanenitrile.

In some of the experiments below, PMA was incorporated into CCNP-Ab to form PMA@CCNP-Ab. Results showed that PMA@CCNP-Ab could significantly increase the population of CD69+ in OT-1 CTLs. The frequencies of IFN-γ- and TNF-α-positive CTLs, and secretion of these cytokines therefrom, were increased further supporting T-cell activation.

### III. PHARMACEUTICAL COMPOSITIONS

Pharmaceutical compositions including the disclosed particles alone or in combination with additional active agents and/or adjuvants are provided. Additionally or alternatively, the pharmaceutical compositions can include cells, e.g., immune cells treated in vitro or ex vivo with the disclosed particles. Pharmaceutical compositions can be for, for example, administration by parenteral (e.g., intramuscular, intraperitoneal, intravenous (IV), intrathecal, or subcutaneous) injection.

In some embodiments, the compositions are administered systemically, for example, by intravenous or intraperitoneal administration, in an amount effective for delivery of the compositions to targeted cells.

In certain embodiments, the compositions are administered locally, for example, by subcutaneous injection, or injection directly into a site to be treated. In some embodiments, the compositions are injected or otherwise administered directly to one or more tumors. Typically, local injection causes an increased localized concentration of the compositions which is greater than that which can be achieved by systemic administration, and/or may reduce toxicity to other tissues (e.g., non-tumor cells). In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can effect a sustained release of the particles to the immediate area of the implant.

The particles, for example nanoparticles, can be provided to the cell either directly, such as by contacting it with the cell, or indirectly, such as through the action of any biological process. For example, the particles, for example nanoparticles, can be formulated in a physiologically acceptable carrier or vehicle, and injected into a tissue or fluid surrounding the cell.

### A. Formulations for Parenteral Administration

In a preferred embodiment the compositions are administered in an aqueous solution, by parenteral injection.

The formulation can be in the form of a suspension or emulsion. In general, pharmaceutical compositions are provided including effective amounts of particles optionally include pharmaceutically acceptable diluents, preservatives, solubilizers, emulsifiers, adjuvants and/or carriers. Such compositions can include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions., or by heating the compositions.

In some embodiments, increasing temperature of a colloidal solution of particles is avoided. In some embodiments, coated nanoparticles can be prepared in a thin film, which can optionally undergo heating. For example, phospholipid can be mixed with nanoparticles in organic solvents such as chloroform. After evaporating chloroform, a thin film is left on the vessel interior surface. Nanoparticles can be shipped in this manner. Before treatment, water/buffer solutions are added to the vessel to redisperse nanoparticles in aqueous solutions.

### B. Other Formulations

The particles can also be applied topically. Topical administration can include application to the lungs, nasal, oral (sublingual, buccal), vaginal, or rectal mucosa. These methods of administration can be made effective by formulating the particles with transdermal or mucosal transport elements. In particular embodiments, the route of administration is nasal administration.

A wide range of mechanical devices designed for pulmonary delivery of therapeutic products can be used, including but not limited to, nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa can be incorporated into a tablet, gel, capsule, suspension or emulsion. Standard pharmaceutical excipients are available from any formulator.

Oral formulations may be in the form of chewing gum, gel strips, tablets, capsules, or lozenges. Oral formulations may include excipients or other modifications to the particle which can confer enteric protection or enhanced delivery through the GI tract, including the intestinal epithelia and mucosa (see Samstein, et al., Biomaterials, 29 (6): 703-8 (2008).

Transdermal formulations may also be prepared. These will typically be ointments, lotions, sprays, or patches, all of which can be prepared using standard technology. Transdermal formulations can include penetration enhancers.

### C. Adjuvants

Adjuvants are known in the art and can be used in the disclosed compositions and methods. The adjuvant may be without limitation alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene] (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Adjuvants may be TLR ligands, such as those discussed above. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-gamma), macrophage colony stimulating factor, and tumor necrosis factor.

### IV. METHODS OF USE

The disclosed compositions can be used in vitro, ex vivo, or in vivo to increase immune responses. Calcium signaling is involved in the activation of different immune cells, including dendritic cells, T cells, macrophages, natural killer cells, and neutrophils. Thus, the disclosed compositions can be used to target these cells to modulate immune responses by them, e.g., by increasing calcium signaling therein. T cells include, for example, effector T cells (e.g., cytotoxic, helper, regulatory, or a combination thereof), memory T cells, Gamma-delta T cells (γδ T cells), Follicular helper T cells (Tfh), Natural killer T cells (NKT cells). In some embodiments, the compositions target a specific cell type. In some embodiments, the compositions target immune cells more generally, and thus target two or more different immune cell types.

In some embodiments, the compositions (e.g., through increased calcium signaling) improve one or more activities of the immune cells. Such activities include, but are not limited to, T cell activation and/or localization to a tumor site, and/or improve dendritic cell (DC) infiltration into a tumor site and/or DC maturation.

In some embodiments, the particles are utilized to activate or prime immune cells, including, but not limited to, antigen presenting cells and/or effector immune cells, in vitro or ex vivo. Such cells can be administered to subject in need thereof to cancer or infections.

A. In vivo Methods

In some embodiments, the particles are utilized to activate or prime immune cells in vivo.

The disclosed compositions can be administered in an effective amount to induce, increase or enhance an immune response. The “immune response” typically refers to responses that induce, increase, induce, or perpetuate the activation or efficiency of innate or adaptive immunity. The composition can be delivered parenterally (by subcutaneous, intradermal, or intramuscular injection) through the lymphatics, or by systemic administration through the circulatory system.

For example, in some embodiments, the compositions are administered to a subject in need thereof, to improve dendritic cell (DC) infiltration into a tumor site and/or DC maturation in the subject.

Additionally or alternatively, the compositions can be administered to a subject in need thereof, to improve T cell activation and/or numbers at a tumor, particularly cytotoxic T cell in the subject.

In the experiments below, AnCHNPs were injected at 200 μg/kg, or ~4 μg per mice. Nanoparticles at such a low doses have no tumoricidal effects on their own. Thus, in some embodiments, the compositions are administered in an amount or manner sufficient to induce an immune response without having a direct antitumor effect.

In some embodiments, the compositions are delivered non-systemically. In some embodiments, the composition is delivered locally, for example, by subcutaneous injection. In some embodiments, the composition is administered at a site adjacent to or leading to one or more lymph nodes which are close to the site in need of an immune response (i.e., close to a tumor or site of infection). In some embodiments, the composition is injected into the muscle. The composition can also be administered directly to a site in need of an immune response (e.g., a tumor or site of infection).

The immune response can be induced, increased, or enhanced by the composition compared to a control, for example an immune response in a subject induced, increased, or enhanced in the absence of the particles. Thus, the compositions and methods can be used to induce or increase an immune activating immune response.

Disclosed compositions can enhance the activity of dendritic cells (DCs). In some embodiments, the immune response includes increase in NF-κB signaling, cytokine activity and immune response in DCs. In some embodiments, particles induce DCs to express or secrete chemokines (e.g. CXCL-1, CCL5, CXCL2 and CXCL10) and cytokines (e.g. IL-1β, IL-12, and IL-6), which are known to attract and stimulate immune cells including T cells. Some embodiments include an increase in phospho-NF-κB, indicating the activation of the NF-κB pathway, and/or increased expression levels of calcineurin and de-phosphorylated NFAT.

The experiments below show that overall, sustained release of calcium from CHNPs leads to activation of both the NF-κB and NFAT pathways, inducing chemokines, cytokines, antigen-presenting, and costimulatory molecules, thereby enhancing DC-mediated immunity.

Disclosed compositions can also enhance the activity of T cells. Thus, in some embodiments, the compositions additionally or alternatively reduce inactivation and/or prolong activation and/or tumor infiltration and/or numbers of T cells (i.e., increase antigen-specific proliferation of T cells, enhance cytokine production by T cells, stimulate differentiation effector functions of T cells and/or promote T cell survival) or overcome T cell exhaustion and/or anergy and/or increase CTL/Treg ratios. In some embodiments, the compositions increase expression and/or secretion of CD69, IFN-γ, and/or TNF-α by T cells.

For example, the experiments below also show that T cells efficiently internalized PMA@CCNP-Ab, resulting in increased intracellular calcium levels, and delivery of calcium and PMA to T cells promoted their activation as evidenced by increased expression or secretion of CD69, IFN-γ, and TNF-α. In vivo testing in B16-OVA tumor-bearing C57/BL6 mice showed that PMA@CCNP-Ab resulted in enhanced tumor infiltration by cytotoxic T cells and increased CTL/Treg ratios. Therapeutic benefits associated with PMA@CCNP-Ab's ability to enhance T cell activation were observed.

The compositions can be administered as part of prophylactic vaccines or immunogenic compositions which confer resistance in a subject to subsequent exposure to cancer antigens or infectious agents, or as part of therapeutic vaccines, which can be used to initiate or enhance a subject's immune response to a pre-existing antigen, such as a viral antigen in a subject infected with a virus or with cancer.

The desired outcome of a prophylactic or therapeutic immune response may vary according to the disease or condition to be treated, or according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent.

Similarly, immune responses against cancer or infectious agents may completely treat a disease, may alleviate symptoms, or may be one facet in an overall therapeutic intervention against a disease.

### B. In Vitro and Ex Vivo Methods

In some embodiments, the method is one of adaptive cell therapy (ACT). For example, methods of adoptive cell therapy are known in the art and used in clinical practice. Generally adoptive cell therapy involves the isolation and ex vivo expansion of tumor specific cells to achieve greater number of cells than what could be obtained by vaccination alone. The tumor specific cells are then infused into patients with cancer in an attempt to give their immune system the ability to overwhelm remaining tumor via cells which can attack and kill cancer. Several forms of adoptive T cell therapy can be used for cancer treatment including, but not limited to, culturing tumor infiltrating lymphocytes or TIL; isolating and expanding one particular T cell or clone; using T cells that have been engineered to recognize and attack tumors (i.e., chimeric antigen receptor (CAR) cells. Additionally or alternatively, antigen presenting cells such as DCs can used as vaccine carriers or antigen-presenting cells (APCs) to prime naive T cells ex vivo or in vivo. Cytotoxic T lymphocytes (CTLs) and natural killer (NK) cells are used as major tool effector cells for ACT. See, e.g., Abaksuchina, et al., Vaccines (Basel), 2021 Nov. 19; 9(11):1363. doi: 10.3390/vaccines9111363.

Thus, in some methods, the disclosed particles are used to prime or activate T cells (e.g., cytotoxic, helper, regulatory, or a combination thereof), memory T cells, Gamma-delta T cells (γδ T cells), Follicular helper T cells (Tfh), Natural killer T cells (NKT cells), dendritic cells, and/or other immune cells in vitro or ex vivo, and later administer them to a subject in need thereof, such as a subject with cancer or an infection. In some embodiments, the cells are harvested from the subject, e.g., directly from the patient's blood, prior to ex vivo treatment with the disclosed particles. Methods of priming and activating T cells in vitro/ex vivo for adaptive T cell cancer therapy are known in the art. See, for example, Wang, et al., Blood, 109(11):4865-4872 (2007) and Hervas-Stubbs, et al., J. Immunol., 189(7):3299-310 (2012). The methods can be used in conjunction with the disclosed compositions and methods to increase the activation of the cells of the adoptive therapy, e.g., dendritic cells, T cells, etc.

Antigen-specific T-cell lines can be generated by in vitro stimulation with antigen followed by nonspecific expansion on CD3/CD28 beads. The ability to expand antigen-specific T cells can be assessed using IFN-gamma and granzyme B enzyme-linked immunosorbent spot. The phenotype of the resultant T-cell lines can be evaluated by flow cytometry. Amplification of antigen-specific T cell populations from Peripheral Blood Mononuclear Cells (PBMCs) is usually performed through repeated in-vitro stimulation with optimal length antigenic peptides in the presence of IL-2. Low doses of IL-2 (between 10 and 50 U/ml) have been used traditionally to avoid the activation/expansion of lymphokine-activated killer cells, as revealed in chromium release assays that were commonly employed to monitor specific T cell expansion. Concentrations of antigenic peptides can be 0.1-10 μM.

Historically, adoptive T cell therapy strategies have largely focused on the infusion of tumor antigen specific cytotoxic T cells (CTL) which can directly kill tumor cells. However, CD4+ T helper (Th) cells can also be used. Th can activate antigen-specific effector cells and recruit cells of the innate immune system such as macrophages and dendritic cells to assist in antigen presentation (APC), and antigen primed Th cells can directly activate tumor antigen-specific CTL. As a result of activating APC, antigen specific Th1 have been implicated as the initiators of epitope or determinant spreading which is a broadening of immunity to other antigens in the tumor. The ability to elicit epitope spreading broadens the immune response to many potential antigens in the tumor and can lead to more efficient tumor cell kill due to the ability to mount a heterogeneic response. In this way, adoptive cell therapy can be used to stimulate endogenous immunity.

Thus, in some embodiments, the compositions administered to a subject in need thereof (e.g., a subject with cancer or an infection), is a population of cells treated in vitro or ex vivo with the disclosed particles.

In some embodiments, ex vivo primed dendritic cells are administered as part of dendritic cell vaccine. Dendritic cell vaccines are a cross between a vaccine and a cell therapy. Due to their proficiency at antigen presentation, DCs are important directors of the induction of antitumor immunity. Dendritic cells can be used as a vaccine by preparing them together with either peptides or small portions of tumor antigens and they can then be injected into the body. DC activation can be particularly strong when the DC vaccine is injected intra-tumorally, and data indicates that the combination of DC-based vaccination with other cancer therapies may further increase the potential of DC-based cancer vaccines and improve patient survival. See also, e.g., Calmeiro, et al., Pharmaceutics. 2020 February; 12(2): 158.

### C. Combination Therapies

The compositions e.g., including particles and/or cells can be administered before, during or after a dose of cancer therapy. The subject can have a benign or malignant tumor. In some embodiments, the subject has cancer and is undergoing cancer therapy, for example, vaccination, radiation therapy, chemotherapy or immunotherapy.

In some embodiments, the compositions enhance the treatment of the cancer compared to administration of the vaccination, radiation, chemotherapy or immunotherapy alone. Administration of the compositions in combination with radiation and/or chemotherapy may enhance the treatment of the cancer compared to administration of radiation and chemotherapy without administration of the composition. Administration of the compositions in combination with radiation and immunotherapy may enhance the treatment of the cancer compared to administration of radiation and immunotherapy without administration of the composition. Administration of the compositions in combination with immunotherapy and chemotherapy may enhance the treatment of the cancer compared to administration of immunotherapy and chemotherapy without administration of the composition. Administration of the compositions in combination with radiation, chemotherapy and immunotherapy may enhance the treatment of the cancer compared to administration of radiation, chemotherapy and immunotherapy without administration of the composition

Thus, in some embodiments, the subject is one that is receiving a radiation-based therapy, including, but not limited to ionizing radiotherapy, phototherapy, or proton therapy.

Thus, the methods include administering the subject one or more doses of ionizing radiation therapy, phototherapy, or proton therapy. Typically, a dose of ionizing, phototherapy or proton therapy radiation is administered (e.g., minute(s), hour(s), or day(s)) after administration of a pharmaceutical composition including the disclosed composition. For example, in exemplary embodiments, a dose of radiation is administered 1 hour to 48 hours, or 1 hour to 24 hours, or 1 hour to 12 hours, or 1 hour to 6 hours, or 2 hours to 6 hours, or 1, 2, 3, 4, or 5 hours before administration of the disclosed pharmaceutical compositions.

In some embodiments, the subject is one that is receiving a chemotherapy.

In some embodiments, the subject is one that is receiving immunotherapy. The subject can be a subject that is receiving only one, or a combination of these therapies.

In some embodiments, the subject is one that is receiving a vaccination, e.g., antigen alone or in combination with an adjuvant.

### D. Subjects to Be Treated

The disclosed compositions including, e.g., particles and/or cells can be administered to a subject in need thereof. In some embodiments, the methods are to treat cancer or an infection. Thus, in some embodiments, the subject has cancer or an infection.

### 1. Cancer

The compositions are useful for treating cancer. In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth. The examples below indicate that the viruses and methods are useful for treating cancer, particular brain tumors, in vivo.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. The compositions are particularly effective in treating carcinomas. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, cancers such as vascular cancer such as multiple myeloma, adenocarcinomas and sarcomas, of bone, bladder, brain, breast, cervical, colo-rectal, esophageal, kidney, liver, lung, nasopharangeal, pancreatic, prostate, skin, stomach, uterine, and germ cell cancers. In some embodiments, the compositions are used to treat multiple cancer types concurrently. The compositions can also be used to treat metastases or tumors at multiple locations.

A representative but non-limiting list of cancers that can be treating using the disclosed compositions include cancers of the blood and lymphatic system (including leukemias, Hodgkin's lymphomas, non-Hodgkin's lymphomas, solitary plasmacytoma, multiple myeloma), cancers of the genitourinary system (including prostate cancer, bladder cancer, renal cancer, urethral cancer, penile cancer, testicular cancer,), cancers of the nervous system (including mengiomas, gliomas, glioblastomas, ependymomas) cancers of the head and neck (including squamous cell carcinomas of the oral cavity, nasal cavity, nasopharyngeal cavity, oropharyngeal cavity, larynx, and paranasal sinuses), lung cancers (including small cell and non-small cell lung cancer), gynecologic cancers (including cervical cancer, endometrial cancer, vaginal cancer, vulvar cancer ovarian and fallopian tube cancer), gastrointestinal cancers (including gastric, small bowel, colorectal, liver, hepatobiliary, and pancreatic cancers), skin cancers (including melanoma, squamous cell carcinomas, and basal cell carcinomas), breast cancer (including ductal and lobular cancer and triple negative breast cancers), and pediatric cancers (including neuroblastoma, Ewing's sarcoma, Wilms tumor, medulloblastoma).

### 2. Infections

The compositions are also useful for treating acute or chronic infectious diseases. Because viral infections are cleared primarily by T-cells, an increase in T-cell activity is therapeutically useful in situations where more rapid or thorough clearance of an infective viral agent would be beneficial to an animal or human subject. Thus, the compositions can be administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) viral infections. For example, pharmaceutical formulations including the composition can be administered topically to treat viral skin diseases such as herpes lesions or shingles, or genital warts. The composition can also be administered to treat systemic viral diseases, including, but not limited to, AIDS, influenza, the common cold, or encephalitis.

Representative infections that can be treated, include but are not limited to infections cause by microorganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Histoplasma, Hyphomicrobium, Legionella, Leishmania, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Plasmodium vivax, Trypanosoma brucei, Entamoeba histolytica, Toxoplasma gondii, Trichomonas vaginalis and Schistosoma mansoni.

In some embodiments, the type of disease to be treated or prevented is a chronic infectious disease caused by a bacterium, virus, protozoan, helminth, or other microbial pathogen that enters intracellularly and is attacked, e.g., by cytotoxic T lymphocytes.

In a preferred embodiment, infections to be treated are chronic infections cause by a hepatitis virus, a human immunodeficiency virus (HIV), a human T-lymphotrophic virus (HTLV), a herpes virus, an Epstein-Barr virus, or a human papilloma virus.

This invention can be further understood by the following numbered paragraphs:
1. A nanoparticle including a calcium core and a shell and/or a coating.
2. The nanoparticle of paragraph 1 wherein the core further includes hydroxide, and optionally is calcium hydroxide (Ca(OH)₂).
3. The nanoparticle of paragraph 1, wherein the core further includes carbonate, and optionally is calcium carbonate (CaCO₃).
4. The nanoparticle of paragraph 1, wherein the core is selected from calcium citrate (CaCit), calcium phosphate (Ca₃(PO4)₂), CaCL₂, calcium sulfate (CaSO₄), CaC₂O₄, Ca(NO₃)₂, calcium silicate (Ca₂SiO₄), calcium fluoride (CaF₂), CaBr₂, and CaI₂.
5. The nanoparticle of any one of paragraphs 1-4 including the shell.
6. The nanoparticle of paragraph 5, wherein the shell reduces, prevents, or otherwise delays degradation of the nanoparticle.
7. The nanoparticle of paragraphs 5 or 6, wherein the shell includes one or more of silica, mesoporous silica, carbon, a sulfide optionally ZnS, CoS, CuS, Cu2S, FeS, MoS, Al2S3, Y2S3, or MnS; an oxide optionally Fe3O4, Fe₂O₃, Gd2O3, TiO2, Al₂O₃, or MnO2; a fluoride optionally NaYF4, YF3, LaF3, CeF3, PrF3, or GdFe3; a fatty acid optionally oleic acid, myristic acid, palmitic acid, palmitoleic acid, stearic acid, oleic acid, linoleic acid, arachidic acid, eicosapentaenoic acid (EPA), or docosahexaenoic acid (DHA); an alkyl amine optionally octylamine, nonylamine, decylamine, undecylamine, laurylamine, tridecylamine, tetradecylamine, pentadecylamine, hexadecylamine, heptadecylamine, octadecylamine, oleylamine; MgO, CuO, or ZnO.
8. The nanoparticle of any one of paragraphs 1-7, wherein the nanoparticle includes the coating.
9. The nanoparticle of paragraph 8, wherein the coating improves dispersion in aqueous solutions and/or delays core release and/or improves half-life.
10. The nanoparticle of paragraphs 8 and 9, wherein the coating includes one or more polymers, peptides, proteins, lipids, or a combination thereof.
11. The nanoparticle of any one of paragraphs 8-10, wherein the coating includes PEG.
12. The nanoparticle of any one of paragraphs 1-11 including a targeting agent optionally wherein the target agent targets one or more immune cells, optionally wherein the one or more immune cells is selected from dendritic cells, T cells, macrophages, natural killer cells, neutrophils, and combinations thereof optionally wherein the T cells are selected from cytotoxic, helper, regulatory, memory T cells, gamma-delta T cells (γδ T cells), follicular helper T cells (Tfh), natural killer T cells (NKT cells), and combinations thereof.
13. The nanoparticle of paragraph 12, wherein the target agent targets dendritic cells.
14. The nanoparticle of paragraphs 12 and 13, wherein the target agent targets CD205 and optionally is an anti-CD205 antibody.
15. The nanoparticle of paragraph 12, wherein the targeting agent targets T cells.
16. The nanoparticle of paragraph 15, wherein the T cells include or are cytotoxic T cells.
17. The nanoparticle of paragraphs 15 or 16, wherein the targeting agent targets CD3 or PD-1, and optionally is an anti-CD3 or anti-PD-1 antibody.
18. The nanoparticle of any one of paragraphs 1-17, further including an active agent, optionally selected from an antigen, chemotherapeutic drug, immune system modulator, immune checkpoint modulator, or an immune cell modulator.
19. The nanoparticle of paragraph 18, including an immune cell modulator optionally wherein the immune cell modulator is a Protein Kinase C (PKC) antagonist optionally wherein the PKC antagonist is phorbol 12-myristate 13-acetate (PMA).
20. A pharmaceutical composition including the nanoparticles of any one of paragraphs 1-19.
21. The pharmaceutical composition of paragraph 20 further including an adjuvant.
22. The pharmaceutical composition of paragraphs 20 or 21 further including an antigen, chemotherapeutic drug, immune system modulator, immune checkpoint modulator, or immune cell modulator.
23. A pharmaceutical composition including immune cells treated in vitro or ex vivo with the nanoparticles of any one of paragraphs 1-19 optionally wherein the immune cells are selected from dendritic cells, T cells, macrophages, natural killer cells, neutrophils, and combinations thereof optionally wherein the T cells are selected from cytotoxic, helper, regulatory, memory T cells, gamma-delta T cells (γδ T cells), follicular helper T cells (Tfh), natural killer T cells (NKT cells), and combinations thereof.
24. A method of increasing calcium signaling in an immune cell including contacting the immune cell with an effective amount of the pharmaceutical composition of any one of paragraphs 20-22 to increase calcium signaling therein, optionally wherein the immune cells is selected from dendritic cells, T cells, macrophages, natural killer cells, neutrophils, and combinations thereof, optionally wherein the T cells are selected from cytotoxic, helper, regulatory, memory T cells, gamma-delta T cells (γδ T cells), follicular helper T cells (Tfh), natural killer T cells (NKT cells), and combinations thereof.
25. A method of enhancing an immune response in a subject in need thereof including administrating the subject an effective amount of the pharmaceutical composition of any one of paragraphs 20-23.
26. The method of paragraph 25, wherein the immune response includes one of more of increasing NF-κB signaling and/or cytokine activity in dendritic cells, improved dendritic cell infiltration into a tumor site, and/or improved dendritic cell maturation.
27. The method of paragraphs 25 or 26, wherein the immune response includes one or more of inducing dendritic cells to express or secrete chemokines (e.g. CXCL-1, CCL5, CXCL2 and/or CXCL10), cytokines (e.g. IL-1B, IL-12, and/or IL-6), or a combination thereof.
28. The method of paragraphs 26 or 27, wherein the immune response includes one or more of increased T cell activation, increased T cell localization to a tumor site, increased expression and/or secretion of CD69, IFN-γ, and/or TNF-α by T cells.
29. The method of any one of paragraphs 24-28, wherein the subject has cancer or an infection.
30. A method of treating or preventing cancer including administering a subject in need thereof an effective amount of the pharmaceutical composition of any one of paragraphs 20-23.
31. The method of paragraph 30, wherein the amount or means of administration is effective to induce an immune response against the cancer but not to have a direct anticancer effect.
32. A method of treating or preventing an infection including administering a subject in need thereof an effective amount of the pharmaceutical composition of any one of paragraphs 20-23.
33. The method of any one of paragraphs 25-32, further including treating the subject with one or more of surgery, radiotherapy, chemotherapy, or immunotherapy optionally an immune checkpoint modulator, an immune system modulator, or immune cell modulator.

This invention can be further understood by way of the following non limiting examples.

### EXAMPLES

### Example 1: Calcium Nanoparticles Stimulate Dendritic Cells and Boot Antitumor Immunity

### Materials and Methods

### Synthesis of Calcium Hydroxide or Ca(OH)2 Nanoparticles (CHNPs)

In a typical synthesis, 443.92 mg of calcium chloride (CaCl2, anhydrous, 97%, Sigma-Aldrich, Lot #SLBQ3073V) was first dissolved in 18.571 mL Milli Q H2O. Into the solution, 1.429 mL of 6 M sodium hydroxide (NaOH, Fisher, Lot #166374) was dropwise added. The resulting solution was stirred magnetically at 90° C. for 5 mins. The raw products were collected by centrifugation and then redispersed in ethanol (200 proof, Koptec, Lot #274014) with brief sonication. The washing step was repeated 3 times to remove unreacted precursors.

### Synthesis of Silica-Coated Calcium Hydroxide Nanoparticles (SCHNPs)

Fifty mg of CHNPs were dispersed in a mixture solvent containing 40 mL ethanol and 0.4 mL ammonia (28.0-30.0%, J.T.Baker, Lot #0000010971). The solution underwent vigorous stirring for 30 mins. After sonication for 30 secs, 300 μL of TEOS (tetraethyl orthosilicate, 98%, Sigma-Aldrich, Lot #STBJ8253) was dropwise added into the solution, followed by the addition of 180 μL of APTES ((3-aminopropyl)triethoxysilane, 98%, Sigma-Aldrich, Lot #MKCM7627). The resulting solution underwent stirring at room temperature for 20 hrs. SCHNPs were collected by centrifugation and washed three times with ethanol.

### Synthesis of PEG-Diacid Coated Calcium Hydroxide Nanoparticles (PCHNPs)

Twenty mg of SCHNPs were dispersed in 10 mL DMSO (dimethyl sulfoxide, 99.9%, Sigma-Aldrich, Lot #MKBF8194V) and transferred to a 20 mL glass vial. Under magnetic stirring, 200 mg PEG-diacid (MW 2,000, JenKem tech, Lot #ZZ192P158), 20 mg EDC (N-(3-Dimethylaminopropyl)-N′-ethylcarbodiimide, 97%, Sigma-Aldrich, Lot #507429), and 15 mg NHS (N-Hydroxysuccinimide, 98%, Sigma-Aldrich, Lot #130672), dissolved in 10 mL DMSO, was added into the nanoparticle suspension. The resulting solution underwent magnetic stirring at 60° C. for 20 hrs. PCHNPs were collected by centrifugation and washed 2 times with Milli Q H2O. Synthesis of Anti-CD205 conjugated calcium hydroxide Nanoparticles (AnCHNPs)

PCHNPs (0.5 mg) were dispersed in 1 mL cold sterile PBS and kept under magnetic stirring at 4° C. Ten μL anti-CD205 antibodies (mouse monoclonal HD30, Sigma-Aldrich, Lot #531834) was added into the PCHNP solution. After 25 mins, 2 μL ethanolamine (99%, Sigma-Aldrich, Lot #398136) was added into solution. After reaction for another 5 mins, AnCHNPs were collected by centrifugation and washed with PBS once. Fresh-made AnCHNPs were used for subsequent in vitro and in vivo studies, unless specified otherwise. All nanoparticle doses were expressed as Ca concentrations unless specified otherwise.

### Physiochemical Characterizations of Nanoparticles

Scanning Electron Microscopy (SEM) and Energy Dispersive X-ray Spectra (EDS) elemental mapping images were acquired on a FEI Teneo field emission SEM equipped with an Oxford EDS system. Transmission Electron Microscopy (TEM) was carried out on an FEI Tecnai20 transmission electron microscope operating at an accelerating voltage of 200 kV. High resolution TEM was performed on a Hitachi transmission electron microscope H9500 operating at a 300 kV accelerating voltage. X-ray diffraction (XRD) analysis was carried out on a Bruker D8-Advance system using dried samples placed on a cut glass slide with Cu Kα1 radiation (λ=1.5406 Å). Dynamic Light Scattering (DLS) and zeta potential measurements were carried out on a Malvern Zetasizer Nano ZS system. Fourier-transform infrared (FT-IR) spectra were recorded on a Nicolet iS10 FT-IR spectrometer.

### Nanoparticle Stability and Calcium Release

CHNPs and PCHNPs were dispersed in 100 μL ammonium acetate buffer solutions (pH=5.5 or 7.4) and loaded into a Slide-A-Lyzer™ MINI Dialysis Device (MWCO=2K, Cat #69550, ThermoFisher, US). The dialysis unit was placed into a 5 mL Eppendorf tube containing 4.5 mL of the same ammonium acetate buffer. The tube was placed on a shaker (20 rpm) at room temperature. At different time points (0, 0.25, 0.5, 1, 2, 4, 8, 10, and 24 hrs), 500 μL solution was taken from the Eppendorf tube and its Ca2+ content was measured by a calcium ion-selective electrode (HORIBA LAQUAtwin Ca-11). 500 μL fresh buffer was added back to the Eppendorf tube to keep the total volume at 4.5 mL. All samples were analyzed in triplicates. In addition, TEM images were acquired for PCHNPs taken at 0, 2, 4, 8, 12, 24 hrs.

### Cell Culture

B16F10-OVA cells (murine melanoma) were grown in high glucose DMEM (ATCC® 30-2002TM) supplemented with G418 ingredient. B16F10 cells (murine melanoma) were grown in high glucose DMEM (ATCC® 30-2002TM). Bone marrow derived dendritic cells (BMDCs) were established from germ cells extracted from the bone marrow of C57BL/6 mice and cultured in RPMI-1640 (Corning, 10-040-CV) containing GM-SCF according to a published protocol (Jiang, et al., Advanced Materials 2019, 31 (46), 1904058). MB49 cells (murine bladder carcinoma) were grown in RPMI-1640 (Corning, 10-040-CV). All cell culturing mediums were supplemented with 10% fetal bovine serum (FBS), 100 units/mL of penicillin, and 100 units/mL streptomycin (MediaTech, USA). All cells were maintained in a humidified, 5% carbon dioxide atmosphere at 37° C.

### Cell Cytotoxicity

ATPlite-1step luminescence assay kit (PerkinElmer, Lot #107-21051) was used to determine cellular ATP contents following the manufacturer's protocol. BMDCs were seeded into 96-well plates at a density of 1×104 cells per well and incubated overnight. The cells were then treated with CaCl₂) solution, AnCHNPs and SiO₂-PEG shell at a dose range of 0.05-100 μg/mL for 24 hrs. The luminescence intensity of each well was measured on a microplate reader (Synergy Mx, BioTeK) and normalized to that of the control cells.

### Cell Uptake

BMDCs were seeded into 6-well plates at a density of 1×10⁶ cells per well and incubated overnight. The cells were then treated with Cy-5 labeled PCHNPs and AnCHNPs (5 μg/mL) for 2 hrs. Furthermore, different endocytosis inhibitors Sodium azide (NaN₃, 99.5%, Sigma-Aldrich, Lot #S2002), Dynasore (C₁₈H₁₄N₂O₄, 98%, Sigma-Aldrich, Lot #324410), Nystatin (Sigma-Aldrich, Lot #N4014), Chlorpromazine (C₁₇H₁₉ClN₂S·HCl, 98%, Sigma-Aldrich, Lot #C8138) were used. The Fluorescence of Cy-5 taken up by DCs were measured by flow cytometry.

### Lysosomal pH

LysoSensor™ Yellow/Blue DND-160 (PDMPO) kit (Invitrogen, Lot #2174576) was used to investigate lysosomal pH changes after BMDCs taking up AnCHNPs. Briefly, BMDCs were seeded into a 96-well plate at a density of 1×10⁴ cells per well and incubated overnight. At different time points (0, 1, 2, 4, 8, and 24 hrs), incubation medium was taken away, replenished with prewarmed (37° C.) probe-containing (1 μM) medium. Cells were incubated for 5 mins under the same growth condition. Then the loading solution was replaced with fresh culturing medium, and the fluorescence (dual-excitation at 329 and 384 nm and dual-emission at 440 and 540 nm) were measured on a microplate reader (Synergy Mx, BioTeK). In acidic organelles LysoSensor™ Yellow/Blue DND-160 (PDMPO) shows predominantly yellow fluorescence, and in less acidic organelles it emits blue fluorescence. The lysosomal pH can be estimated based on the blue/yellow fluorescence ratio.

### [Ca2+]int Measurement

Fluo-3 AM kit (Cayman, 14960) was used to measure [Ca2+]int in BMDCs after treatment with AnCHNPs. Briefly, BMDCs were seeded into a 96-well plate at a density of 1×10⁴ cells per well and incubated overnight. At different time points (0, 1, 2, 4, 8, and 24 hrs), medium was taken from the well, replenished with prewarmed (37° C.) probe-containing medium (to a final concentration of 5 μM). Cells were incubated for 30 mins under the same growth conditions. Then the loading solution was replaced with fresh medium, removing dye molecules nonspecifically attached to cell surface. Cells were incubated for another 30 mins to allow for complete de-esterification of the acetoxymethyl esters. Fluorescence (ex/em: 485/520 nm) was recorded on a microplate reader (Synergy Mx, BioTeK).

### [Na+]int and [K+]int Measurement

SBFI-AM (sodium-binding benzofuran isophthalate acetoxymethyl ester, Setareh Biotech, Lot No.: 50609), PBFI-AM (potassium-binding benzofuran isophthalate acetoxymethyl ester, Setareh Biotech, Lot No.: 5027) were used to measure [Na+]int and [K+]int in BMDCs respectively after treatment with AnCHNPs, following manufacturer's protocol. Briefly, BMDCs were seeded into a 96-well plate at a density of 1×104 cells per well and incubated overnight. At different time points (0, 1, 2, 4, 8, and 24 hrs), medium was taken from the well, loading solution containing probes (final concentration of 10 μM) were added into wells. Cells were incubated for 30 mins under the same growth conditions. Then the loading solution was replaced with fresh medium, removing dye molecules nonspecifically attached to cell surface. Fluorescence (ex: 340/380 nm, em: 505 nm) was recorded on a microplate reader (Synergy Mx, BioTeK) and the ratio was used to determine the concentration of Na+ and K+ respectively.

### Investigate BMDCs' Maturation, Migration, and Antigen-Presentation In Vitro

Maturation BMDCs were seeded onto a 6-well plate at a density of 1×106 cells per well one day before the experiment. BMDCs were treated with PBS, CaCl2 solution (5 or 10 μg/mL), and AnCHNPs (5 or 10 μg/mL). After incubation for 24 hrs, supernatant was removed, and BMDCs were harvested by cell lifter. BMDCs were subsequently stained with MHCII-FITC (#107616) and CD205-APC (#138206) and analyzed flow cytometry. Also, BMDCs were treated with SiO₂-PEG shell (10 μg/mL), harvested after 24 hrs incubation, stained with MHCII-FITC (#107616), CD80-PerCP-Cy5.5 (#560526), CD86-BV605 (#563055), CD40-PE (#12-0401-83), and OVA-APC (#17-5743-82), and analyzed by flow cytometry.

Migration B16F10-OVA cells after receiving 100 Gy irradiation (320 kv) were transferred into the lower chamber of a 6-well Transwell® Permeable Support system at a density of 1×10⁵ cells per well. For control, un-irradiated B16F10-OVA cells were used. CFSE-labeled BMDCs at a density of 1×10⁶ cells per well were seeded onto the upper chamber of the well. BMDCs were treated with: PBS, CaCl₂) solution (5 or 10 μg/mL), and AnCHNPs (5 or 10 μg/mL). LPS (1 μg/mL) was tested as a positive control (supporting information). After 24 hrs incubation, cells in the lower chamber were harvested by cell lifter and readied for flow cytometry. Percentage of CFSE positive cells was quantified.

### Activation and Antigen-Presentation

Irradiated B16F10-OVA cells (100 Gy, 320 kv) were transferred into a 6-well plate at a density of 1×10⁵ cells per well. For comparison, un-irradiated B16F10-OVA cancer cells were tested. BMDCs at a density of 1×10⁶ cells per well were seeded into each well. The co-cultures were treated with: PBS, CaCl₂) solution (5 or 10 μg/mL), AnCHNPs (5 or 10 μg/mL). After 24 hrs incubation, the cells were harvested by cell lifter, stained with MHCII-FITC (#107616), CD80-PerCP-Cy5.5 (#560526), CD86-BV605 (#563055), CD40-PE (#12-0401-83), and OVA-APC (#17-5743-82), and analyzed by flow cytometry. Moreover, the supernatant was collected and its IL-6, IL-10, IL-12, TNF-α contents were measured by ELISA using R&D Systems Mouse IL-6, IL-10, IL-12, TNF-α DuoSet kits (Minneapolis, MN). The results were analyzed using the Four Parameter Logistic Curve method by Myassay.com.

### RNA Sequencing (RNA-Seq) and Data Analysis

BMDCs were seeded onto a 100 mm petri dish at a density of 1×10⁶ cells per well and incubated overnight. Cells were treated with OVA (10 μg/mL) or OVA (10 μg/mL)+AnCHNPs (5 μg/mL). After incubation for 12 hrs, cells were harvested by a cell lifter. The NucleoSpin® miRNA kit (Takara, Lot #2010/002) was used for extracting RNA from three independent samples of BMDCs with different treatments. RNA quality was analyzed using a 2100 Bioanalyzer (Agilent Technologies, Santa Clara, CA). The purified RNA samples were sent to Novogene Corporation (Sacramento, CA) for library construction and sequencing using the Illumina HiSeq™ 2000 platform to obtain expression libraries of 50-nt read length. RNAseq data were analyzed as previously described. In brief, differentially expressed genes (DEGs) were identified using the DESeq R package functions estimateSizeFactors and nbinomTest. P value <0.05 and fold change >1.5 or fold change <0.5 was set as the threshold for significantly differential expression. Hierarchical cluster analysis of DEGs was performed to explore transcript expression patterns, and Gene Ontology (GO) was performed to identify the potential function of all DEGs. GSEA was conducted using GSEA desktop application software with annotated gene sets of Molecular Signature Database v6.2. The detailed RNA-seq information of this assay is available in GSE208276 deposited in the NIH Gene Expression Omnibus (GEO) database.

### RT-qPCR

RT-qPCR was performed on a QuantStudio 3 system using SYBR Green as an indicator. The PCR reaction mixture included 10 ng of cDNA, 500 nM of each primer (synthesized by Sigma, St. Louis, MO), 5 μL of 2× SYBR Green PCR Master Mix (Quantabio, Cat #101414-284), and RNase-free water which was added to increase the final volume to 10 μL. The qRT-PCR reaction was carried out for 40 cycles at 95° C. for 15 secs and 60° C. for 1 min. The data were quantified based on the ΔΔCt method using GAPDH and histone as internal standards for normalization. Melting curve analysis for all qRT-PCR products was performed which showed a single DNA duplex. Primer sequences are:

NOS2:
For
(SEQ ID NO: 1)
5′-AGAGCCACAGTCCTCTTTGC-3′;
Rev
(SEQ ID NO: 2)
5′-GCTCCTCTTCCAAGGTGCTT-3′.
CCL5:
For
(SEQ ID NO: 3)
5′-CTGCTGCTTTGCCTACCTCT-3′;
Rev
(SEQ ID NO: 4)
5′-CGAGTGACAAACACGACTGC-3′.
CXCL1:
For
(SEQ ID NO: 5)
5′-CTGGGATTCACCTCAAGAACATC-3′;
Rev
(SEQ ID NO: 6)
5′-CAGGGTCAGGCAAGCCTC-3′.
IL-12b:
For
(SEQ ID NO: 7)
5′-ATGAGAACTACAGCACCAGCTTC-3′;
Rev
(SEQ ID NO: 8)
5-ACTTGAGGGAGAAGTAGGAATGG-3′.
IL-1b:
For
(SEQ ID NO: 9)
5′-TCGTGCTGTCGGACCCATAT-3′;
Rev
(SEQ ID NO: 10)
5′-GTCGTTGCTTGGTTCTCCTTGT-3′.

### Western Blot

BMDCs were seeded onto a 100 mm petri dish at a density of 1×10⁶ cells per cell and incubated overnight. The cells were then treated with OVA (10 μg/mL) or OVA (10 μg/mL) plus AnCHNPs (5 μg/mL). After incubation for 24 hrs, cells were harvested and lysed with a RIPA buffer supplemented with 1× proteinase inhibitor cocktail (Amresco). Protein concentration was determined using bicinchoninic acid (BCA) protein assay (Thermo Fisher Scientific). Protein lysates were loaded onto 10% SDS-PAGE and transferred to a PVDF membrane. Nonspecific binding to the membrane was blocked by incubation with 5% nonfat milk at room temperature for 1 h. The membrane was incubated with primary antibodies at the dilutions specified by the manufacturers at 4° C. overnight. This is followed by incubation with secondary antibodies for 1 h at room temperature, and then treatment with ECL reagents (Thermo Fisher Scientific). The membrane was then exposed to X-ray films (Santa Cruz). All the imaging results were analyzed by ImageJ. The antibodies used are: NFAT1 (Cell Signaling Cat #4389S); Pan-Calcineurin A (Cell Signaling Cat #2614S); IκBα, Phospho-IκBα, NF-κB p65, Phospho-NF-κB p65 (Cell Signaling Cat #9936T); GAPDH (Cell Signaling Cat #5174S). Animal Models

All experimental procedures were conducted following a protocol approved by the Institutional Animal Care and Use Committee (IACUC) of the University of Georgia. C57BL/6 mice (female, 4 weeks old) were purchased from the Envigo Laboratories and maintained under pathogen-free conditions. The animal models were established by subcutaneously injecting 2×105 B16F10-OVA, B16F10, or MB49 cells in 50 μL PBS into the right hind limb of each mouse after 2 weeks of settlement (6 weeks old).

### Flow Cytometry to Profile Immune Cells

C57BL/6 mice bearing B16F10-OVA tumors were randomly divided into three groups (n=10 for each group), which were treated with: (1) 10 Gy X-ray irradiation (320 kv)+PBS (50 μL), and (2) 10 Gy X-ray irradiation+CaCl2 solution (200 μg/kg, i.t.), (3) 10 Gy X-ray irradiation+AnCHNPs (200 μg/kg, i.t.). The treatment began when tumor size reached ~100 mm3 (Day 0). All injections were performed at five sites of the tumor to ensure good coverage. CaCl2 and AnCHNPs were injected in 50 μL PBS, 1 h after the radiation. On Day 3, 5 mice from each group were euthanized. The rest of the animals were euthanized on Day 7. Tumor, spleen, and tumor-draining lymph node were harvested for immune response profiling. Tumors were cut into small pieces with scissors and digested by incubating with DMEM containing 1 mg/mL collagenase type V (Worthington Biochemical Corporation) at 37° C. for 45 mins. The digested tissues were gently meshed though a 250 μm cell strainer (Thermo scientific, Lot #UB2685874A). Red blood cells were lysed with Ack lysing buffer (Gibco) according to the manufacturer's instructions. The single-cell suspensions were washed with cold sterile PBS and resuspended in staining buffer. Following counting and aliquoting, cells were stained with fluorophore-conjugated antibodies for 30 mins at 4° C. Spleen and lymph nodes were processed following similar procedures, except that a 70 μm cell strainer (Corning Falcon, Ref #352235) was used and that no collagenase type V was used. The following anti-mouse antibodies from BD Biosciences were used: CD45-APC-Cy7 (#557659), CD4-BV605 (#563151), FoxP3-PE (#563101), CD11c-PE-Cy7 (#558079), CD86-BV605 (#563055), CD80-PerCP-Cy5.5 (#560526). CD40-PE (#12-0401-83) was purchased from Invitrogen. OVA-APC (#17-5743-82) was purchased from eBioscience. MHCII-FITC (#107616), CD205-APC (#138206), IFN-γ-APC (#505810), CD3-FITC (#100206) and CD8-BV510 (#100752) were purchased from BioLegend. Live/dead DAPI was purchased from Thermal Fisher. Multi-parameter staining was used to identify the following cell populations of interest: (a) CD8+ T cells (CD45+CD3+CD8+), (b) CD8+IFNγ+ T cells (CD45+CD3+CD8+IFNγ+), (c) CD4+ T cells (CD45+CD3+CD4+), (d) Treg cells (CD45+CD3+CD4+FoxP3+), (e) MHC-II+ DCs (CD11c+MHC-II+), (f) CD80+ DCs (CD11c+MHC-II+CD80+), (g) CD86+ DCs (CD11c+MHC-II+CD86+), (h) CD40+ DCs (CD11c+MHC-II+CD40+), (i) OVA+DCs (CD11c+MHC-II+SIINFEKL-H-2Kb+). For intracellular FoxP3 and IFN-γ staining, cells were fixed and permeabilized using Permeabilization Solution Kit (BD, 554714), and washed before flow cytometry (Quanteon, Agilent). For assessing tumor-specific T-cell response, splenocytes were co-cultured with B16F10-OVA cells for 6 hrs before staining and flow cytometry. The data were processed by FlowJo 10.0. Doublets were excluded based on forward and side scatter. Dead cells were excluded based on positive DAPI staining. In addition, blood samples were collected on Day 3 and 7 for cytokine analysis. Specifically, IL-1β, IL-6, IL-10, IL-12, TNF-α, and IFN-γ in the serum were measured using R&D Systems Mouse DuoSet ELISA kits (Minneapolis, MN) following the manufacturer's protocol. Results were analyzed using the Four Parameter Logistic Curve method from Myassay.com.

### Therapy Studies

Combination with radiotherapy (RT): The studies were performed in C57BL/6 mice bearing B16F10-OVA or MB49 tumors. For B16F10-OVA tumor models, when tumor sizes reached ~50 mm3, the animals were randomized to receive the following treatments (n=5 for each treatment group): (1) PBS (i.t., 50 μL*2, Day 0 and Day 2), no irradiation; (2) AnCHNPs (i.t., 200 μg/kg*2, Day 0 and Day 2); (3) RT (10 Gy*2, Day 0 and Day 2)+PBS (i.t., 50 μL*2, Day 0 and Day 2); (4) RT (10 Gy*2, Day 0 and Day 2)+AnCHNPs (i.t., 200 μg/kg*2, Day 0 and Day 2); (5) RT (10 Gy*2, Day 0 and Day 2)+AnCHNPs (i.t., 200 μg/kg*2, Day 0 and Day 2)+anti-CD8 antibodies (i.p., 10 mg/kg*2, Day 0 and Day 4); (6) RT (10 Gy*2, Day 0 and Day 2)+AnCHNPs (i.t., 200 μg/kg*2, Day 0 and 2)+anti-CD4 antibodies (i.p., 10 mg/kg*2, Day 0 and Day 4). All i.t. injections were performed at five sites of a tumor to ensure good coverage. Antibodies and AnCHNPs were injected in 100 μL and 50 μL PBS respectively. AnCHNPs were injected 1 h after radiation if RT was applicable. The tumor size and body weight were inspected daily. Tumors were measured in two dimensions with a caliper and their volumes were calculated using (length)×(width) 2/2. After therapy, tumors and major organs were collected and sectioned into 4-μm-thick slices for H&E and Ki-67 staining. For MB49 tumor models, animals received the following treatments (n=5 in each group): (1) PBS (i.t., 50 μL*2, Day 0 and Day 2), no irradiation; (2) RT (10 Gy*2, Day 0 and Day 2)+PBS (i.t., 50 μL*2, Day 0 and Day 2); (3) RT (10 Gy*2, Day 0 and Day 2)+AnCHNPs (i.t., 200 μg/kg*2, Day 0 and Day 2). The treatment protocols are similar to those described for B16F10-OVA studies.

Combination with chemotherapy: This was investigated in C57BL/6 mice bearing B16F10-OVA tumors. When tumor sizes reached ~50 mm3, the animals were randomized to receive the following treatments (n=5 for each group): (1) PBS (i.t., 50 μL*2, Day 0 and Day 2); (2) Carboplatin (i.p., 40 mg/kg, 100 μL, Day 0); (3) Carboplatin (i.p., 40 mg/kg, 100 μL, Day 0)+AnCHNPs (i.t., 200 μg/kg*2, 50 μL, Day 0 and Day 2). The tumor size and body weight were inspected daily. The tumor was measured in two dimensions with a caliper and tumor volume was estimated as (length)×(width) 2/2.

Combination with immunotherapy: This was investigated in C57BL/6 mice bearing B16F10 tumors. When tumor sizes reached ~50 mm3, the animals were randomized to receive the following treatments (n=5 for each group): (1) PBS (i.t., 50 μL, Day 0 and Day 2); (2) Anti-PD-L1 antibodies (i.p., 10 mg/kg, Day-2, 0, 2 and 4); (3) Anti-PD-L1 antibodies (i.p., 10 mg/kg, Day-2, 0, 2 and 4)+AnCHNPs (i.t., 200 μg/kg, Day 0 and Day 2). Antibodies were intraperitoneally injected and AnCHNPs were i.t. injected on day 0 and day 2. All injections were performed at five sites of the tumor to ensure good coverage. Antibodies and AnCHNPs were injected in 100 μL and 50 μL PBS respectively. The tumor size and body weight were inspected every other day. The tumor was measured in two dimensions with a caliper and tumor volume was estimated as (length)×(width) 2/2.

### Statistical Analysis

All in vitro studies were performed in triplicates unless specified otherwise. Half-maximum inhibitory concentration (IC50) was determined by Doseresp using Origin 9. For in vivo studies, all measurements were performed three times unless specified otherwise. All data were represented as mean±S.D. Comparisons of multiple assays were performed using one-way ANOVA test and comparisons of two groups were performed using a paired t-test, with a P value of 0.05 or less representing statistical significance.

### Results

### Nanoparticle Synthesis, Surface Modification, and Physiochemical Characterizations

Calcium hydroxide nanoparticles (CHNPs) were synthesized through a co-precipitation method using CaCl₂) and NaOH as precursors (FIG. 1A). Scanning electron microscopy (SEM) (FIG. 1B) and transmission electron microscopy (TEM) (FIG. 1C) revealed that CHNPs are hexagonal in shape, with an average diameter (the long diagonal of the hexagons) of 219.9±17.8 nm. X-ray powder diffraction (XRD) confirmed that the nanocrystals are hexagonal-sheet phase Ca(OH)2 (PDF #01-073-5492, FIG. 1E).

CHNPs were then coated with silica (FIG. 1A). A mixture of tetraethyl orthosilicate (TEOS) and (3-aminopropyl)triethoxysilane) (APTES) was used as silane precursors so that the resulting nanoparticles present amine groups on the surface. Polyethylene glycol (PEG) diacid was subsequently conjugated (m.w.=2000) onto the surface of silica through EDC/NHS coupling. SEM and energy dispersive spectroscopy (EDS) confirm the successful coating (FIG. 1d&f). TEM reveals that the coating thickness is ~20 nm (FIG. 1C). XRD shows that the coating did not negatively affect the crystallinity of the Ca(OH)2 core (FIG. 1E).

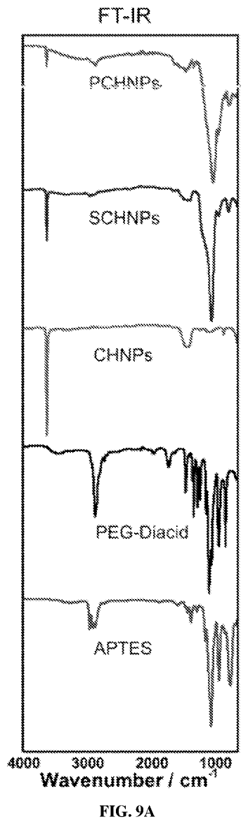

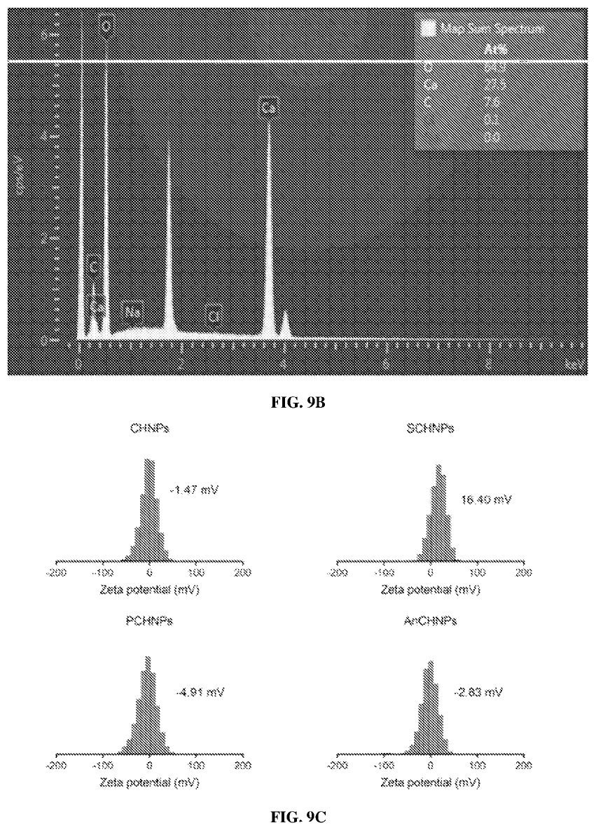

The PEGylated Ca(OH)₂/SiO2 core/shell nanoparticles (PCHNPs) can be well dispersed in water. Their hydrodynamic size is 245.2±30.26 nm, compared to 227.3±27.02 nm for bare Ca(OH)₂/SiO₂ nanoparticles (FIG. 1G). The surface of PCHNPs was almost neutral (−4.91 mV, FIG. 1H). As a comparison, bare Ca(OH)₂/SiO₂ nanoparticles are slightly positively charged (+16.4 mV) due to surface amine groups. Successful PEGylation was also confirmed by Fourier-transform infrared (FT-IR), finding characteristic stretching (2882 cm−1) and bending (1467 and 1341 cm⁻¹) peaks of C—H, as well as the stretching peak of C—O—C(10³³ cm⁻¹) with PCHNPs (FIG. 9A).

Lastly, anti-CD205 antibodies were coupled onto PSCHNPs using EDC/NHS chemistry. The resulting conjugates, i.e. AnCHNPs, were stable in aqueous solutions (FIG. 1I). By quantifying protein and calcium, it is estimated that each nanoparticle carries on average 27 antibody molecules. Coupling with antibodies increased the hydrodynamic size of the nanoparticles to 295.3±46.7 nm (FIG. 1G). Meanwhile, the surface charge was slightly increased to −2.83 mV over the conjugation (FIG. 1H).

In summary, Ca(OH)₂ nanoparticles were synthesized, coated with silica, and PEGylated on the surface. Anti-CD205 antibodies were successfully conjugated onto the nanoparticles.

AnCHNPs Uptake by DCs and their Impact on [Ca²⁺]ᵢₙₜ

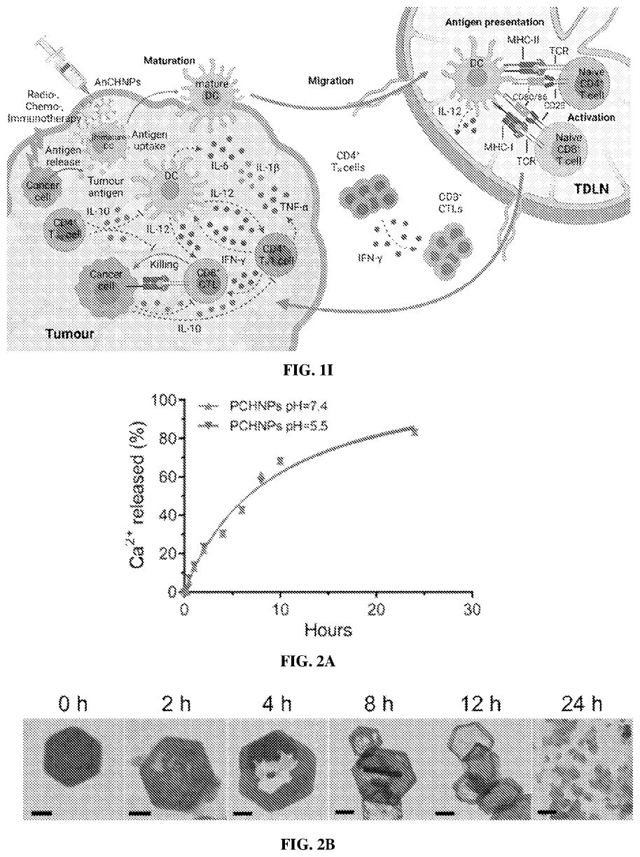

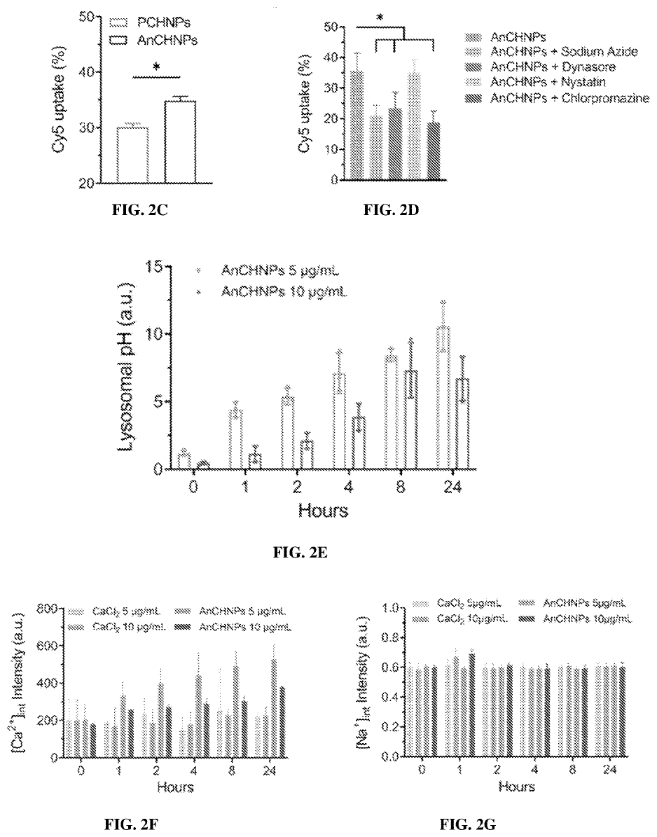

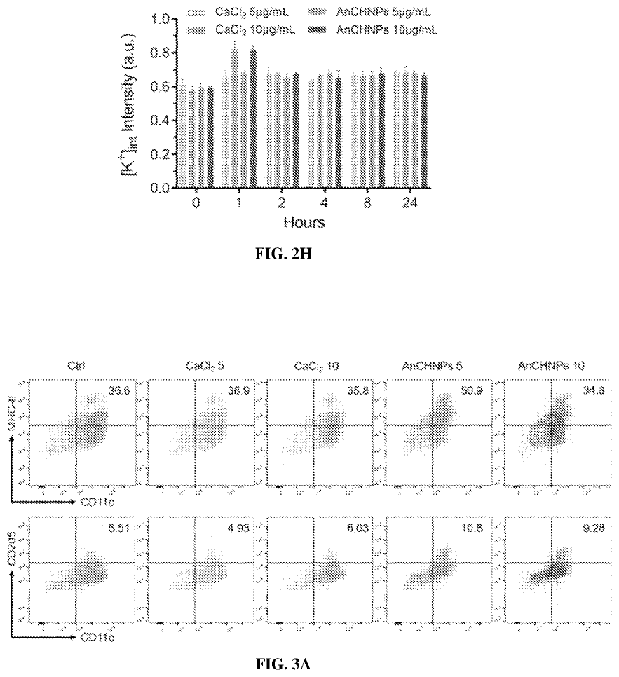

The silica coating slows down but does not prevent the Ca(OH)₂ core from degradation. A sustained calcium release from PCHNPs was observed in buffer solutions at neutral pH (FIG. 2A). The accumulative release reached ~80% at 24 h, with a half-life of ~7 h (FIG. 2A). The degradation rate barely changed when the pH of the solution was reduced to 5.5. Samples taken from PCHNPs solutions at different times were also examined under TEM. In accordance with the release results, there was a gradual dissolution of the Ca(OH)₂ core (FIG. 2B). Meanwhile, the silica shell remained largely intact, effectively functioning as a capsule for calcium.

Subsequent experiments studied cellular uptake of AnCHNPs by BMDCs. To this end, AnCHNPs were labelled with Cy5 and incubated with BMDCs (Bone marrow derived dendritic cells) at 5 or 10 μg/mL (based on Ca, the same below). For comparison, Cy5-labeled PCHNPs were also tested. Flow cytometry found significantly increased nanoparticle uptake with AnCHNPs relative to PCHNPs (FIG. 2C). The uptake was reduced when AnCHNPs were co-incubated with azide, a general inhibitor of endocytosis. The uptake was also inhibited by chlorpromazine and dynasore (FIG. 2D), which blocks clathrin- and dynamin-dependent endocytosis, respectively. Meanwhile, nystatin, which inhibits the caveolae endocytosis pathway, had no effect on the particle uptake. These results indicate that AnCHNPs enter DCs through receptor-mediated endocytosis, which was observed by others with anti-CD205 antibodies (Tel, et al., European journal of immunology 2011, 41 (4), 1014-1023, Schreibelt, et al., Blood, The Journal of the American Society of Hematology 2012, 119 (10), 2284-2292).

Incubation with AnCHNPs caused an increase of lysosomal pH (FIG. 2E), likely due to proton neutralization by Ca(OH)2. Meanwhile, Fluo-3AM assay found time-dependent increase of [Ca²⁺]int (FIG. 2F). This is owing to the degradation of Ca(OH)2 particles and in parallel, an release of calcium into the cytosol. The [Ca²⁺]int increase lasted more than 24 h, which coincides with what was observed in solutions. As a comparison, CaCl₂ salt at the same calcium doses induced little [Ca²⁺]int increase (FIG. 2F). Meanwhile, [Na⁺]ᵢₙₜ and [K⁺]ᵢₙt levels remained largely unchanged according to SBFI-AM and PBFI-AM assays (FIGS. 2G & 2H).

Overall, the results confirmed that AnCHNPs are taken up by DCs through clathrin- and dynamin-dependent endocytosis and gradually degrade inside the cells to enable a sustained [Ca²⁺]ᵢₙₜ increase.

### Impact of AnCHNPs on DC Maturation and Migration

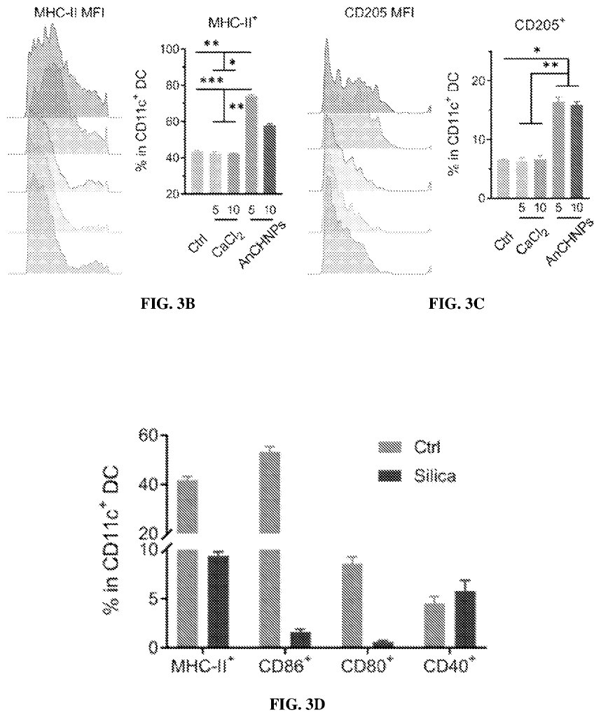

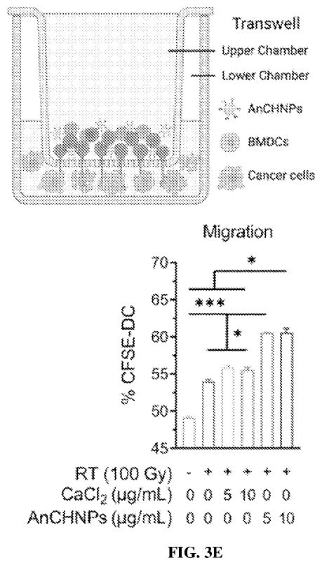

AnCHNPs were first incubated with BMDCs at 5 or 10 μg/mL in the absence of cancer cells and surface MHC-II analyzed by flow cytometry (FIG. 3A). Relative to untreated DCs, both the population and expression levels of MHC-II⁺ DCs (MFI) were significantly increased when BMDCs were treated with AnCHNPs (FIG. 3B), indicating enhanced DC maturation. AnCHNPs also induced CD205 expression in DCs (FIG. 3C), which potentially creates a positive feedback loop causing more AnCHNPs uptake, furthering cell maturation. Note that upregulation of CD205 in activated DCs was reported by others (Butler, et al., Immunology 2007, 120 (3), 362-371). As a comparison, CaCl₂ had no impact on either MHC-II or CD205 expression (FIG. 3A-3C). Silica nanoparticles of similar sizes to AnCHNPs also showed no positive impact on MHC-II expression (FIG. 3D).

Subsequent studies examined the impact of AnCHNPs on DC migration in a transwell assay, where B16F10-OVA cells, with or without irradiation (100 Gy), were seeded onto the lower chamber and CFSE-labeled BMDCs loaded onto the insert. Compared to un-irradiated B16F10-OVA cells, those receiving irradiation led to enhanced transwell migration of DCs (FIG. 3E), due to radiation-induced release of DAMPs and chemokines that promote chemotactic movement (Randolph, et al., Annual review of immunology 2008, 26 (1), 293-316). Incubation with AnCHNPs significantly increased the numbers of DCs migrating to the bottom chamber, indicating that the nanoparticles can boost DCs' ability to sense chemotactic signals and move towards the source. As a comparison, CaCl₂ minimally affected DC migration.

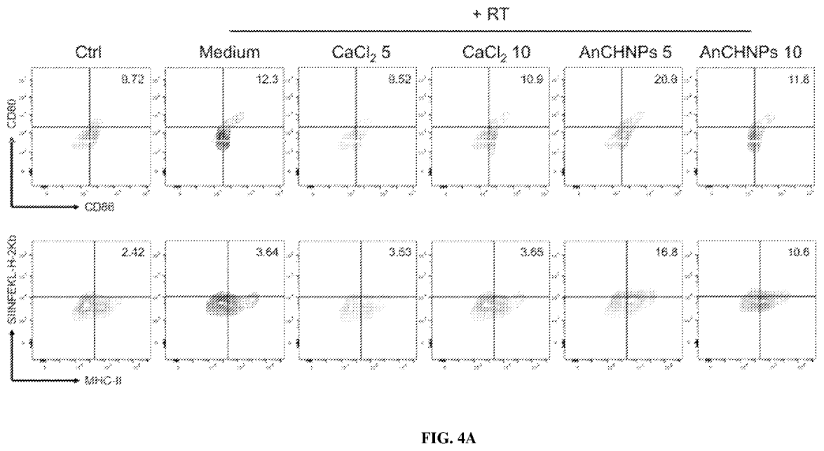

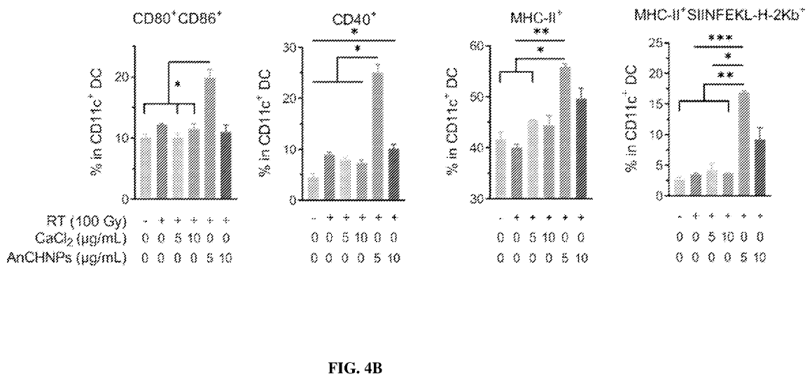

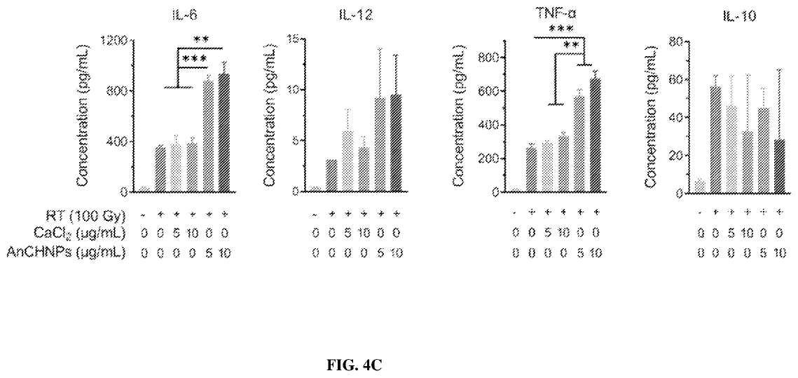

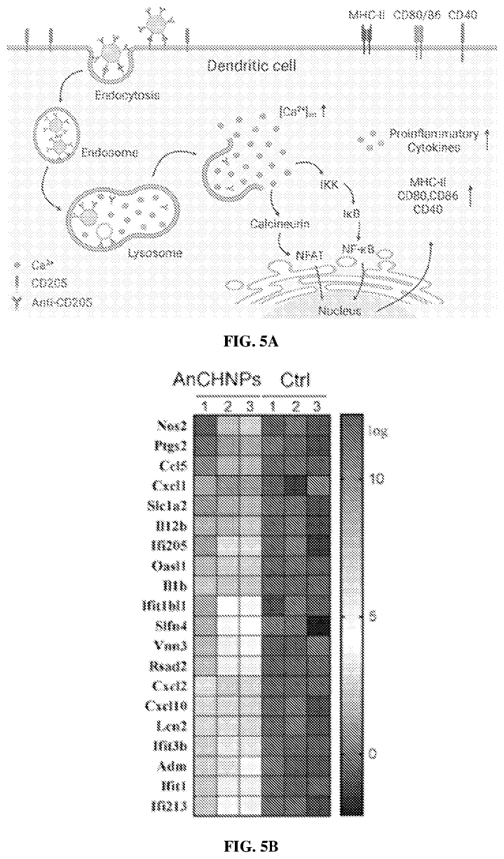

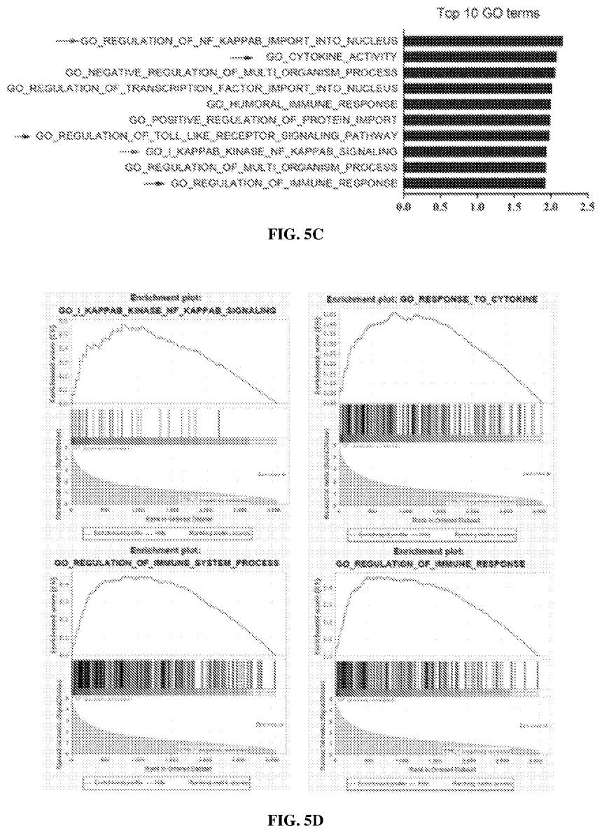

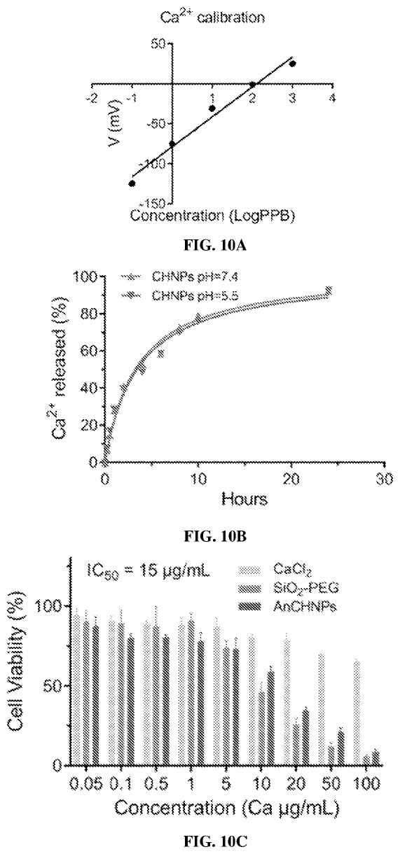

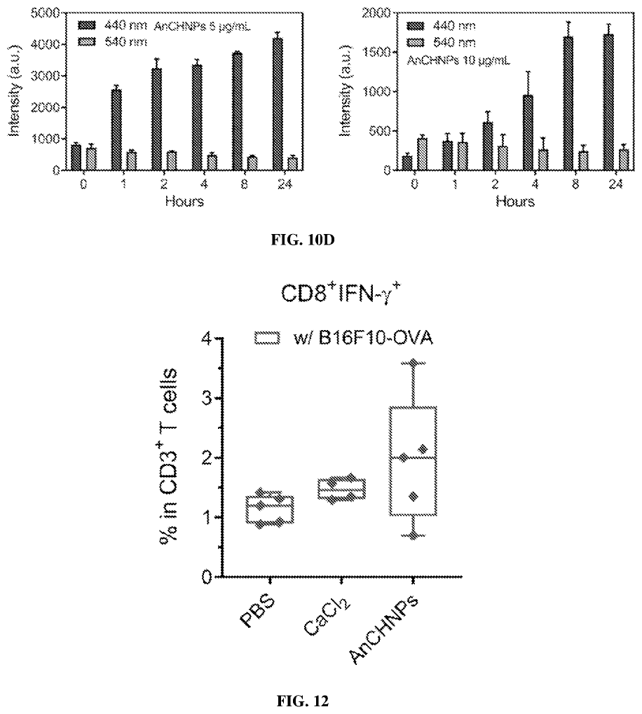

Next, DC maturation and activation when co-culturing them with pre-irradiated (100 Gy) B16F10-OVA cells was examined. Treatment with AnCHNPs significantly increased the frequencies of CD80⁺CD86⁺ DCs in this setting (FIG. 4A). Other maturation markers, including CD40 and MHC-II, were also elevated (FIG. 4B). In addition, there was a significant increase of surface SIINFEKL-H-2Kb, indicating enhanced antigen presentation of DCs when treated with AnCHNPs. Of note, AnCHNPs worked more efficiently at 5 μg/mL than at 10 μg/mL, which was likely due to negative impact of nanoparticles on cell viability at the higher concentration (FIG. 10C). As a comparison, calcium salt and silica nanoparticles had no positive impact on DC activation (FIG. 3D). Cytokines in the supernatant of the co-cultures were also measured. Relative to DCs treated with carrier only or CaCl₂), those treated with AnCHNPs showed elevated secretion of pro-inflammatory cytokines, including IL-6, IL-12, and TNF-α (FIG. 4C), but a reduced secretion of IL-10 (though not significant, p=0.3307), an anti-inflammatory cytokine.

Overall, these in vitro results support the conclusion that AnCHNPs effectively enhance maturation, migration, and antigen presentation of DCs.

### Mechanisms Underlying DC Activation by AnCHNPs

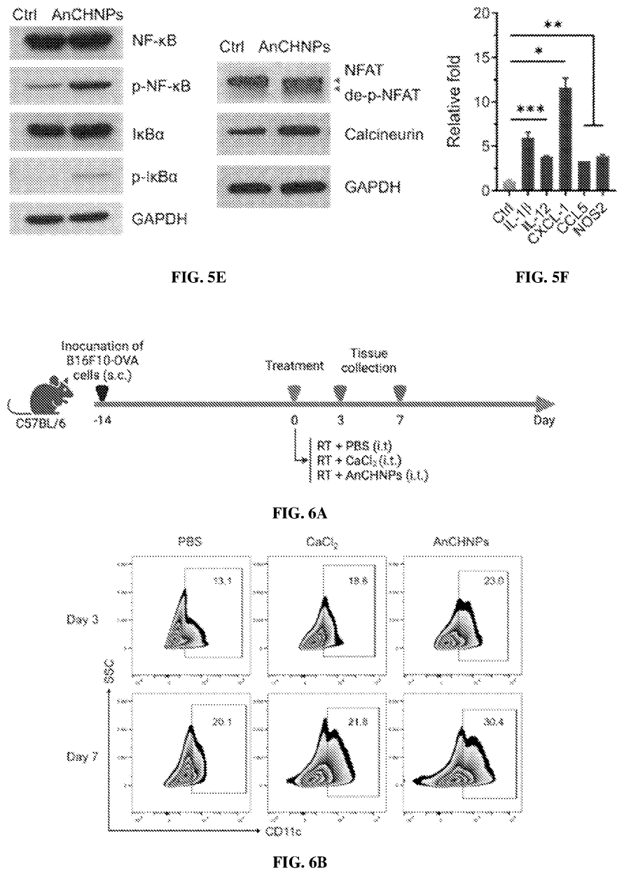

To explore changes in gene expression occurring in DC cells with or without AnCHNPs, out whole-transcriptome sequencing studies were conducted. DEGs analysis showed that 1325 genes (fold change >1.5 and P <0.05) were upregulated and 3049 genes (fold change <0.5 and P<0.05) were downregulated in AnCHNP-treated mouse BMDCs. Interestingly, nitric oxide synthase 2 (Nos2), a reactive free radical acting as a biologic mediator in antitumoral activity, was the most upregulated gene in BMDCs following AnCHNP treatment (FIG. 5B). GO enrichment analysis revealed that gene signatures of NF-κB signaling, cytokine activity and immune response were among the top 10 most upregulated GO terms in AnCHNP-treated BMDCs compared with the control (FIG. 5C). Consistently, GSEA analysis also showed that I_KAPPAB_KINASE_NF_KAPPAB_SIGNALING, RESPONSE_TO_CYTOKINE, REGULATION_OF_IMMUNE_SYSTEM_PROCESS, and REGULATION_OF_IMMUNE_RESPONSE were mostly enriched in BMDCs in the presence of AnCHNP (FIG. 5D). These observations were validated by qPCR, which found that treatment with AnCHNPs induce chemokines (e.g. CXCL-1, CCL5, CXCL2 and CXCL10) and cytokines (e.g. IL-1β, IL-12, and IL-6), which are known to attract and stimulate immune cells including T cells (FIG. 5F). Western blotting was also performed to investigate the activation pathways of BMDCs. Relative to controls, BMDCs treated with AnCHNPs showed an increased expression of phospho-NF-κB, indicating the activation of the NF-κB pathway. Meanwhile, AnCHNPs treatment also led to increased expression levels of calcineurin and de-phosphorylated NFAT, indicating the activation of the NFAT axis (FIG. 5E).

Overall, sustained release of calcium from AnCHNPs leads to activation of both the NF-κB and NFAT pathways, inducing chemokines, cytokines, antigen-presenting, and costimulatory molecules, thereby enhancing DC-mediated immunity.

### Impact of AnCHNPs on Immune Responses In Vivo

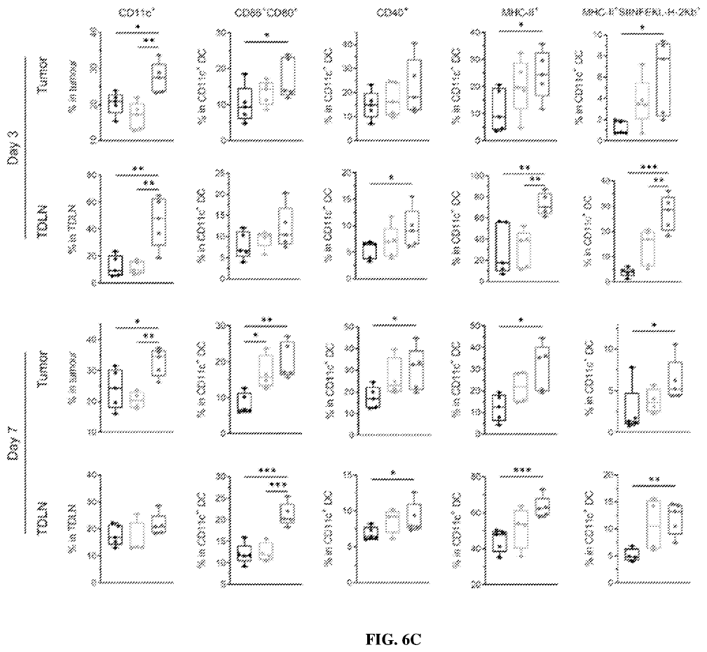

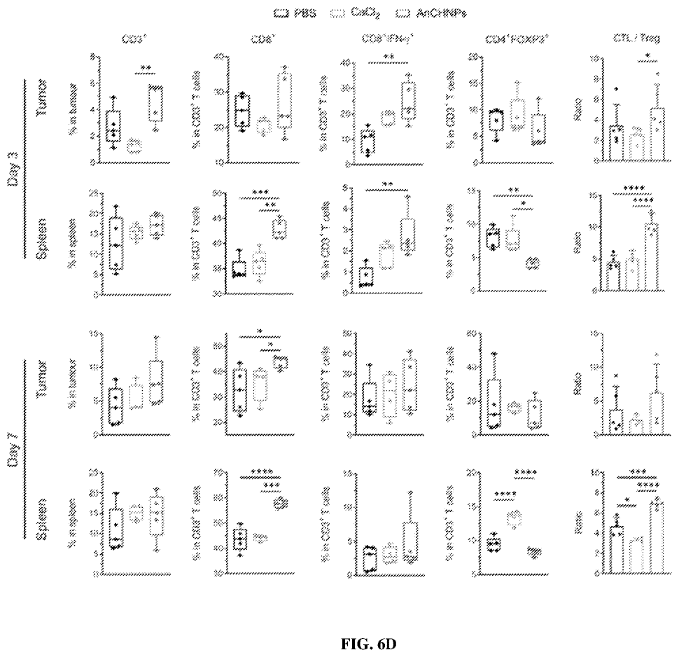

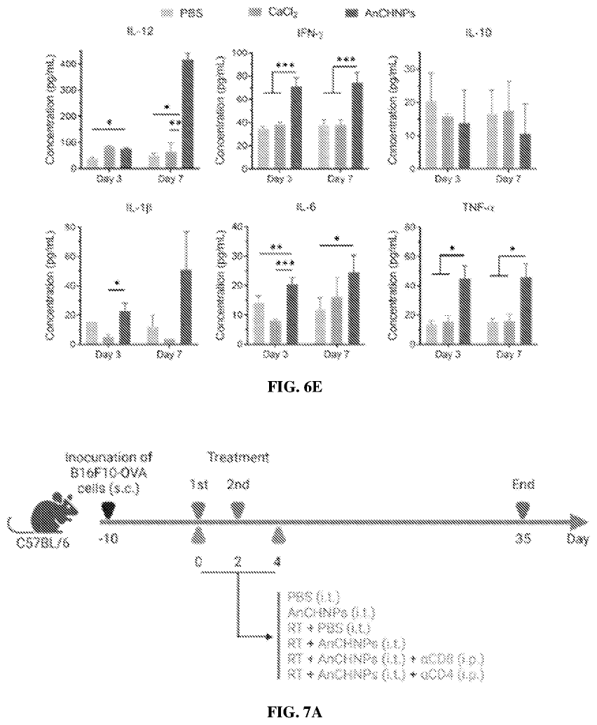

Subsequent studies set out to investigate the impact of AnCHNPs in vivo. This was tested in B16F10-OVA-tumor-bearing C57BL/6 mice. Radiation (10 Gy) was applied to tumors, which presumably triggered antigen/DAMP release. This was followed by intratumoral (i.t.) administration of AnCHNPs (200 μg/kg) at 1 h. For comparison, CaCl2 or carrier only (PBS) was injected. Animals were euthanized on Day 3 or 7, and the tumor, spleen, and tumor-draining lymph nodes (TDLNs), harvested, for flow cytometric analysis (FIG. 6a).

Compared to the PBS or CaCl₂ controls, mice treated with AnCHNPs showed a significant increase of CD11c+ cells in tumors on both Day 3 and 7, indicating elevated tumor infiltration of DCs (FIG. 6B). In particular, populations of the MHC-II+, CD80+CD86+, and CD40+ DCs were significantly increased (FIG. 6C), indicating enhanced DC maturation. Moreover, AnCHNPs caused an increase of SIINFEKL-H-2Kb+ DCs in tumors on Day 3, indicating improved antigen presentation (FIG. 6C). Similarly, increased populations of MHC-II+, CD80+CD86+, CD40+, and SIINFEKL-H-2Kb+ DCs was observed in TDLNs on Day 3 (FIG. 6c), which is attributed to enhanced DC migration over AnCHNPs treatment.

T lymphocytes in tumors were also examined. AnCHNPs significantly promoted tumor-infiltration of cytotoxic T cells (CTLs, CD45+CD3+CD8+) on Day 7. In particular, the population of effector T cells (IFN-γ+CTLs) was elevated on both Day 3 and 7 (FIG. 6D). Meanwhile, the frequency of Tregs (CD45+CD3+CD4+Foxp3+) was significantly reduced. The tumor CTL/Treg ratio was increased by ~2 folds in the AnCHNP group, indicating a strong boost of intratumoral immunity. Similar trends were also observed among T lymphocytes in the spleen (FIG. 6D). As a comparison, CaCl2 had a minimum impact on either CTLs or Tregs in tumors.

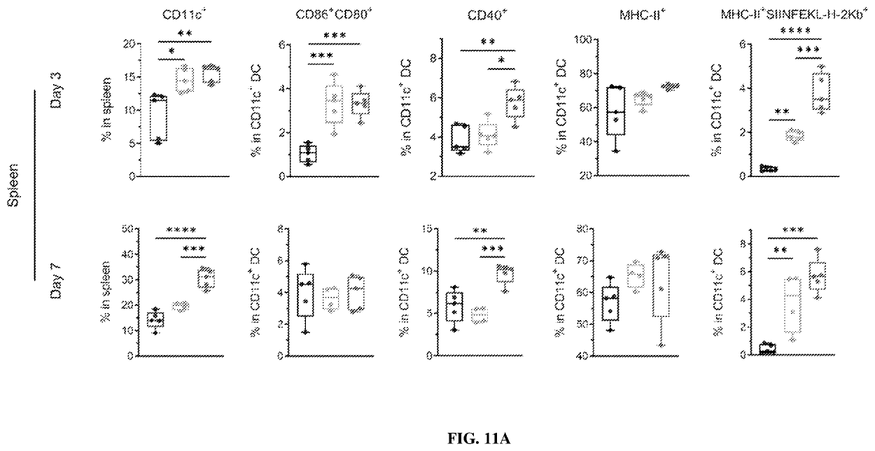

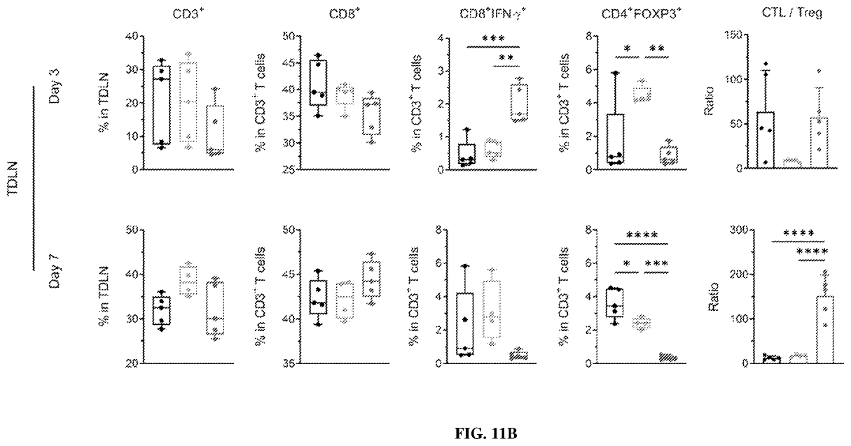

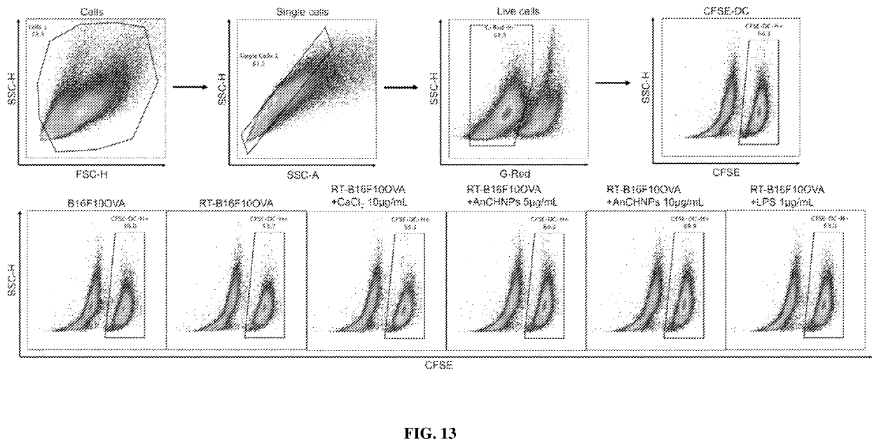

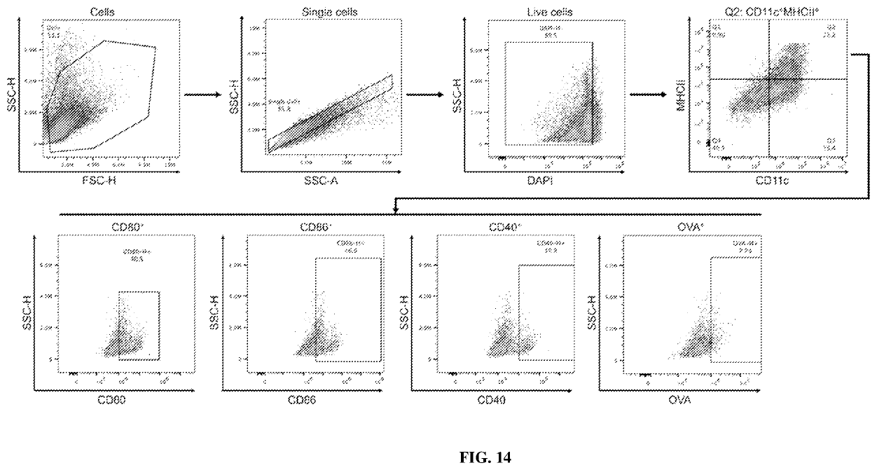

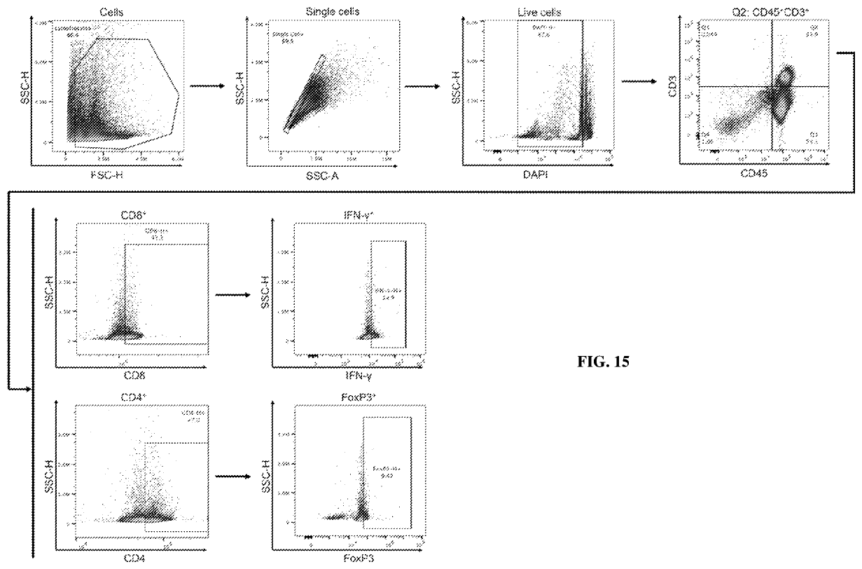

Antigen specific cellular immunity was also examined by co-incubating splenocytes with B16F10-OVA cells ex vivo. In splenocytes taken from the AnCHNPs-treated group, there was a significant increase of IFN-γ+ CTL frequency (FIG. 12), indicating that the nanoparticles elicited a systemic anti-tumor immune response. Conversely, splenocytes taken from the CaCl2 group showed marginal T cell activation over the co-incubation.

Serum from different treatment groups was examined for cytokine levels. Relative to the PBS control, animals treated with AnCHNPs, but not CaCl2, showed elevated levels of IL-1β, IL-6, TNF-α, IFN-γ, and IL-12 but a decreased level of IL-10, on both Day 3 and Day 7 (FIG. 6E), results of which echo with the leucocyte profiling studies.

Overall, the results show that AnCHNPs can promote DC maturation and migration, in turn augmenting both innate and cellular immunity against cancer.

Evaluate the Efficacy of AnCNHPs when Used in Combination of RT

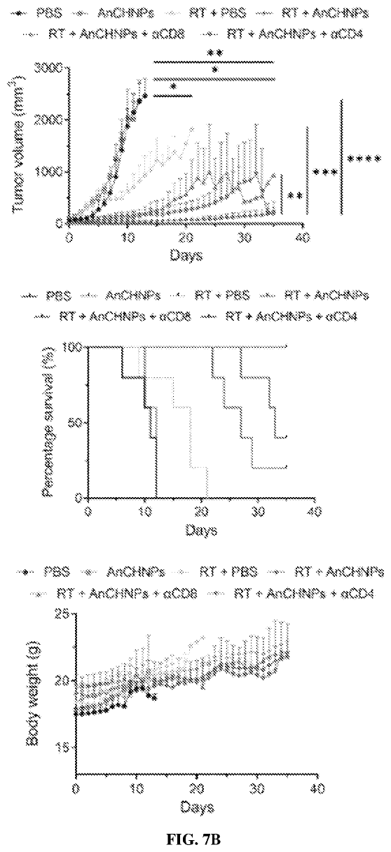

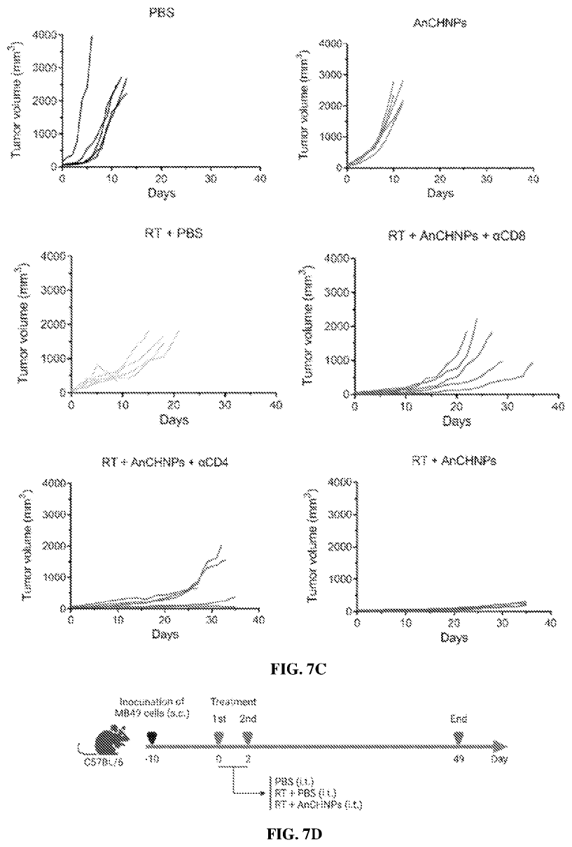

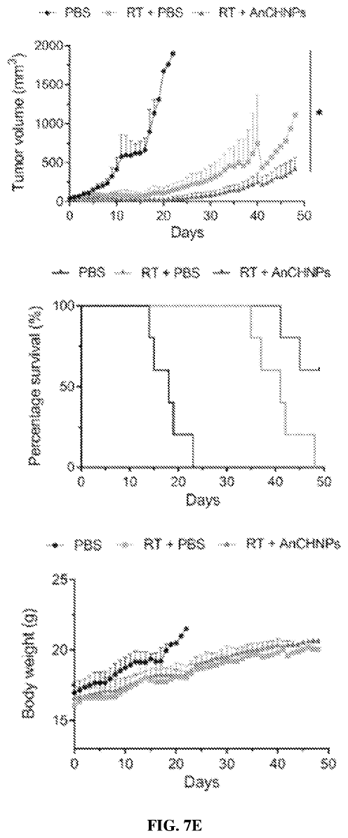

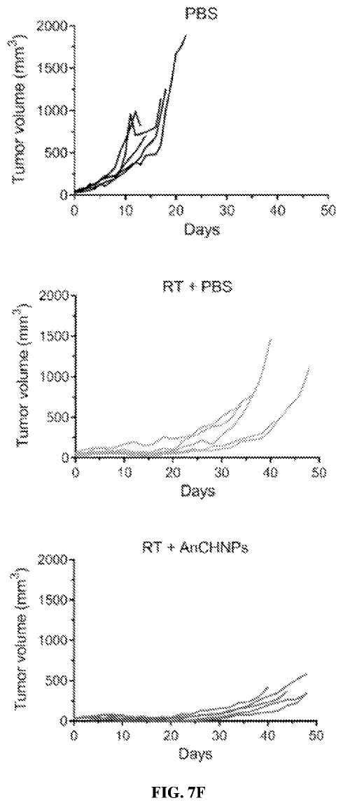

Next, the therapeutic benefits of AnCNHPs when used in combination with other treatments was assessed, beginning with RT. This was also tested in the B16F10-OVA tumor model. Specifically, AnCHNPs (50 μL, 200 μg/kg, in PBS were i.t. injected one hour after radiation (10 Gy) was applied to the tumor, with the rest of the body lead-shielded. A total of two sessions were given two days apart (RT+AnCHNPs). For comparison, animals were treated with carrier only, RT only, or AnCHNPs only (FIG. 7A).

Tumors in the PBS group grew rapidly, with all the animals either dying or reaching a humane endpoint by 2 weeks (FIG. 7B). RT moderately inhibited tumor growth, but all animals in the group died within 3 weeks. As a comparison, AnCHNPs plus RT significantly improved tumor suppression. Eighty percent of the animals in the combination group experienced tumor regression in the first three weeks. All animals in the group remained alive after 5 weeks, 20% of which were tumor-free. Of note, AnCHNPs only had no impact on tumor growth (FIGS. 7B & 7C), indicating that the therapeutic benefits are attributed to the nanoparticles' immunomodulatory effects. This notion is supported by results from T cell depletion studies, where animals received either anti-CD4 or anti-CD8 antibodies in addition to the AnCHNPs-RT combination. Either CD4 or CD8 T cell depletion worsened the treatment outcomes. Between the two, anti-CD8 antibodies more significantly abrogated the therapeutic benefits. These results support a conclusion that boosted cellular immunity is a major factor behind AnCHNPs' radiosensitizing effects (FIG. 7B & 7C).

Post-mortem histopathology was performed on tumor and major organ specimens. Hematoxylin/eosin (H&E) staining exhibited large areas of nuclear shrinkage and fragmentation in tumors treated with AnCHNPs plus radiation. This is accompanied with a reduced level of positive Ki-67 staining in the combination group, indicating decreased cell proliferation. Meanwhile, no signs of toxicity were observed in all major organ tissues.

To validate, AnCHNPs plus RT was also tested in MB49-tumor-bearing C57BL/6 mice (FIG. 7D). RT alone was more effective in this model, extending the average survival from 17 days to 40 days. Adding AnCHNPs to the regimen significantly improved the efficacy. Relative to RT alone, tumor growth suppression was improved by 65.9% in the combination group on Day 40. Sixty percent of the animals remained alive after 7 weeks, while all animals in the PBS and RT group had died at the time (FIGS. 7E & 7F).

In summary, the in vivo studies show that a small dose of AnCHNPs were able to effectively amplify RT-induced immunity, leading to improved tumor control and animal survival.

Evaluate the Efficacy of AnCNHPs when Used in Combination with Chemotherapy or Immunotherapy

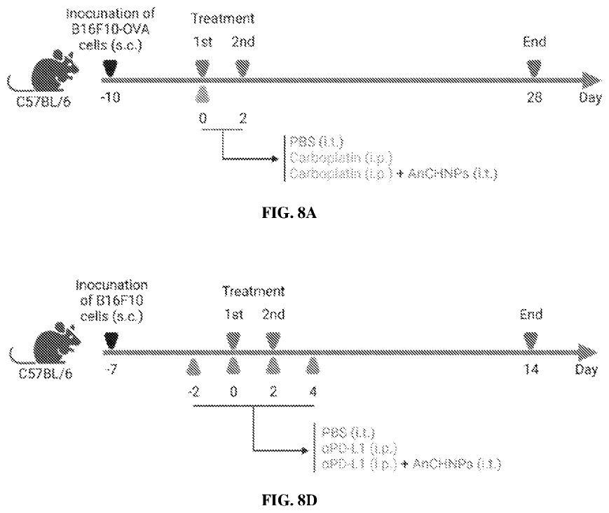

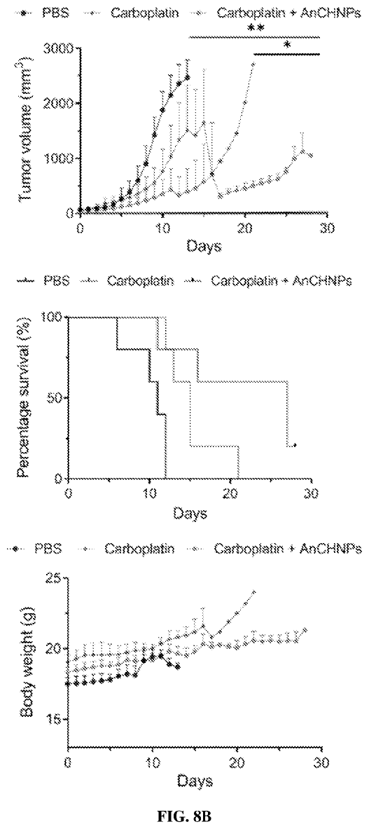

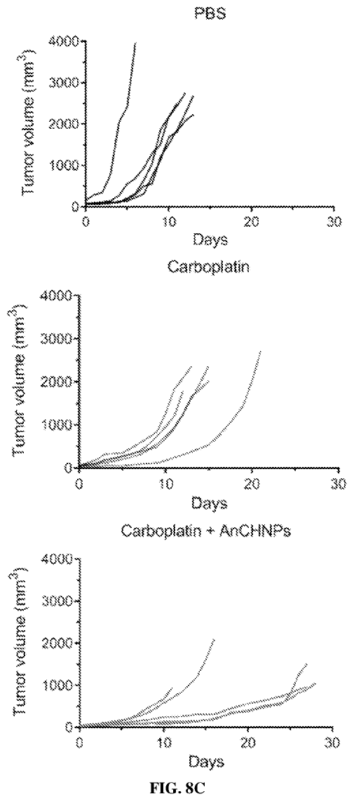

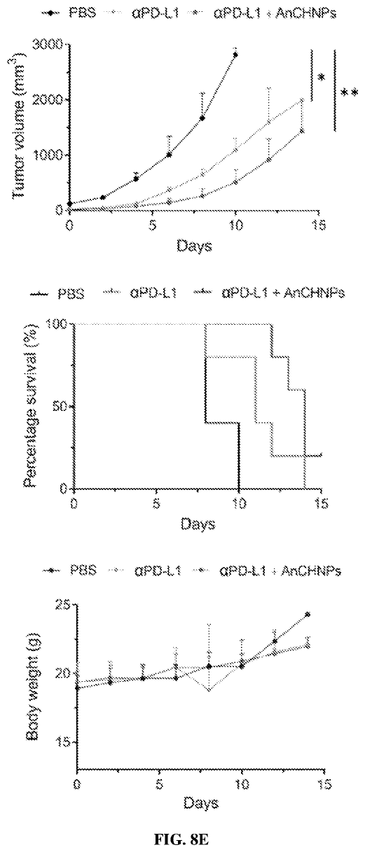

Next studies evaluated whether AnCNHPs can augment the efficacy of chemotherapy such as carboplatin. This was first tested in B16F10 tumor models using a combination of carboplatin (40 mg/kg, i.p.) and AnCHNPs (200 μg/kg, i.t.) (FIG. 8A). Carboplatin is a known ICD agent, but as a monotherapy it is inefficient to elicit a robust immunity (Ho, et al., Critical reviews in oncology/hematology 2016, 102, 37-46). Indeed, carboplatin alone only marginally retarded tumor growth (FIG. 8B), with all animals died within 3 weeks. Adding AnCHNPs significantly improved the treatment outcomes, extending the average survival from 15 days in the carboplatin group to 23 days in the combination group. No additional toxicities were observed.

Studies also investigated whether AnCHNPs can enhance the efficacy of immune checkpoint blockade, also in B16F10 tumor bearing mice (FIG. 8D). B16F10 is a poorly immunogenic tumor model (Yang, et al., Journal of nanobiotechnology 2021, 19 (1), 1-11) and anti-PD-L1 antibodies alone (10 mg/kg, 4 times) showed only moderate therapeutic benefits. Adding AnCNHPs (200 μg/kg, i.t.) to the regimen led to improved efficacy (FIG. 8E & 8F), and the combination was well tolerated.

Overall, the results show that AnCHNPs can also enhance the efficacy of chemotherapy and immunotherapy without causing additional toxicity.

### SUMMARY

In the current study, AnCHNPs was explored as an immunomodulatory agent. AnCHNPs enter cells through endocytosis, and degrade inside the lysosomes, releasing calcium into the cytosol. Normally, DCs are activated by sensing external stimuli such as pathogens or damaged tissues by pattern recognition receptors (e.g. Toll-like receptors). This would trigger a cascade of events that lead to calcium store depletion, activation of the Ca²⁺ release-activated Ca²⁺ channels, and elevated calcium influx (Shumilina, et al., American Journal of Physiology-Cell Physiology 2011, 300 (6), C1205-C1214). As a comparison, AnCHNPs directly activate the NFAT and NF-κB pathways, resulting in DC maturation even in the absence of an external stimulus (FIG. 3A).

While calcium phosphate nanoparticles have long been used in gene delivery, the impact of calcium nanoparticles on the functions of immune cells, DCs in particular, have seldom been investigated. Several recent studies show that calcium carbonate nanoparticles may boost immune response, but the mechanisms behind the activation remain largely elusive. Moreover, few attempts were made at delivering calcium nanoparticles selectively into DCs. This is important because calcium released to the extracellular environment minimally contribute to |Ca²⁺|ᵢₙₜ increase (FIG. 2F). In other words, calcium nanoparticles delivered to TME but not directly into DCs do not activate the immune cells. Here Calcium nanoparticles were coupled with anti-CD205 antibodies, which promote receptor-mediated endocytosis. The nanoparticles were also coated with silica, which provides sustained calcium release over a rapid dissolution. Both designs contribute to efficient immunomodulation.

One advantage of a calcium-based immunomodulator is its high biocompatibility. As shown in the studies herein, the calcium core of AnCHNPs is largely degraded after 24 h. The resulting calcium ions, which cannot freely go across the plasma membrane, are safely excreted. The risks of local and systemic toxicities are low if not non-existent. For therapy studies, AnCHNPs were injected at 200 μg/kg, or ~4 μg per mice. Nanoparticles at such a low dose have no tumoricidal effects on their own (FIGS. 7B & 7C). On the other hand, the data shows that AnCHNPs alone, at the tested dose, does overcome the immunosuppressive factors in the TME for triggering the innate and adaptive immunities. However, higher doses of AnCHNPs elicit a robust immunity without causing calcium overdose. Tumor-antigens and/or conventional immunomodulators can be loaded onto the disclosed calcium nanoparticles, effectively creating a vaccine that furthers DC-mediated anti-tumor immunity. Overall, the current investigation introduces a nanoplatform that opens opportunities for safe and efficient immunomodulation and cancer management.

### Example 2: Calcium Nanoparticles to Enhance T Cell Immunity and Promote Cancer Treatment

### Materials and Methods

### Nanoparticle Synthesis and Characterization

### Synthesis of CaCO₃ Nanoparticles

To synthesize calcium carbonate (CaCO₃) nanoparticles, a co-precipitation method with calcium chloride and ammonium bicarbonate was used. Specifically, 1359 mg of CaCl₂) was dissolved in 900 mL of ethanol in a 1000 mL glass beaker. To facilitate the dissolution, a water bath sonication process can be used. The beaker was carefully covered with parafilm and pierced evenly with a 29 G needle to allow CO₂ to pass through. The beaker was then placed in a 3 L plastic beaker containing 36 g NH₄HCO₃. The entire reaction system was sealed with parafilm. Particle formation began after about 60 hours. The particle size using dynamic light scattering (DLS) until the desired diameter of 150 to 160 nm was reached. CaCO₃ nanoparticles were collected by centrifugation at 12,096 g for 10 minutes. After centrifugation, the nanoparticle pellets were washed three times with 20 mL of ethanol. The CaCO₃ nanoparticles were dispersed in 10 mL of ethanol and stored at room temperature for future use.

### Synthesis of CaCO₃@OA Nanoparticles

10 mg of CaCO₃ nanoparticles were dispersed in 20 mL of ethanol and 20 mg of oleic acid was added to react overnight at room temperature with constant stirring. CaCO₃@OA nanoparticles were obtained by centrifugation at 12,096 g for 10 minutes. To remove unreacted oleic acid, the particles were washed three times with a mixture of 5 mL ethanol and 10 mL hexane.

### Synthesis and Characterization of CaCO₃@OA@Lipid (CCNP)

The aforementioned CaCO₃@OA nanoparticles are hydrophobic and can be dispersed in hexane. The CaCO₃@OA nanoparticles were coated with PEGylated phospholipids such as 1,2-distearoyl-sn-glycero-3-phosphoethanolamine-N-[carboxy(polyethylene glycol)-2000] (DSPE-PEG-COOH). The coating was introduced by hydrophobic-hydrophobic interaction. After coating, the nanoparticles (CCNP) became hydrophilic and easily dispersed in aqueous solutions.

In a typical reaction, 20 mg of DSPE-PEG-COOH (dissolved in chloroform) was mixed with 10 mg of CaCO3@OA in a 50 mL round bottom flask and sonicated the mixture until the particles were completely dispersed. The chloroform was then removed using a rotary evaporator at room temperature to form a thin film on the bottom of the flask. 20 mL of HEPES buffer (0.01 M) was added to the flask and immediately sonicated in a water bath for 5 minutes. The CCNP were concentrated by centrifugation at 9,400 g for 10 minutes. A second round of centrifugation can be performed to collect additional CCNP.

### Synthesis and Characterization of CCNP-Ab

Anti-PD1 antibodies were conjugated to CCNP using EDC/NHS chemistry. Briefly, 10 mg of CCNP was dispersed in 2.4 mL of HEPES buffer, then 2 mg of EDC (5 mg/mL, HEPES) and 4 mg of NHS sulfo (5 mg/mL, HEPES) were added to the mixture. The solution was vortexed at 220 rpm for 20 minutes, followed by centrifugation at 9,400 g for 10 minutes. The particle pellets were then redispersed in 0.75 mL HEPES. 200 μg anti-PD-1 antibody was added to the solution and agitated for 30 minutes. To quench the reaction, 28 μL ethanolamine solution (200 mg/mL) was added and the mixture was vortexed for another 10 minutes. CCNP-Ab nanoparticles were collected by centrifugation at 9,400 g for 10 minutes, redispersed in 350 μL HEPES, and stored at 4° C. for future use.

### Synthesis of PMA@CCNP-Ab

To incorporate PMA into CCNP-Ab, CCNP-Ab were prepared at the desired calcium concentration in an aqueous solution. PMA (5 mg/mL, acetonitrile) was then added to give a final concentration of 50 ng/mL. The solution was sonicated for 1 minute to complete the loading process. The loading rate was calculated by HPLC.

### Results

The role of the immune system, and cytotoxic T cells in particular, in the fight against cancer is now well established. Cancer cells possess tumor-associated antigens (TAAs) that, like viruses and bacteria, can be recognized by the immune system and killed by cytotoxic T cells (CTLs) in an antigen-specific manner. However, solid tumors are often characterized by an immunosuppressive environment that inhibits T cell activation and proliferation or renders them anergic. Strategies, including immune checkpoint inhibitors (ICIs), are being developed to directly or indirectly enhance the functions of endogenous T cells. However, a significant proportion of patients do not respond to ICIs. Alternatively, antigen-specific T cells can be expanded or engineered outside the patient's body and reintroduced into the host. These include adoptive T cell transfer and CAR-T therapies, which have made significant progress and are entering the clinic. However, the efficacy of these therapies can still be limited by issues such as toxicity and the hostile tumor microenvironment. There is a need for new immunotherapy options that can be used either as a stand-alone treatment or in combination to augment existing immunotherapies.

To solve these problems, innovative calcium nanoparticle-based immunomodulators that specifically targets T cells and enhances T cell function have been developed. Calcium plays a central role in T cell activation as a second messenger. Calcium signaling begins with stimulation of the TCR pathway and ultimately leads to activation of the transcription factor NFAT through activation of the calcium-sensitive phosphatase calcineurin. The technology can deliver calcium, in the form of calcium nanoparticles, directly into the cytosol of T cells, to regulate T cell function. For controlled calcium release, which is important for T cell activation, the calcium nanoparticles were coated with a lipid layer. This coating also allows the loading of additional immunomodulators, such as PKC antagonists like phorbol 12-myristate 13-acetate (PMA), which work with calcium to boost T-cell immunity. In addition, targeting ligands, such as anti-PD1 antibodies, can be conjugated to the nanoparticles to direct the nanoparticles to T cells.

### Nanoparticle Synthesis and Characterization

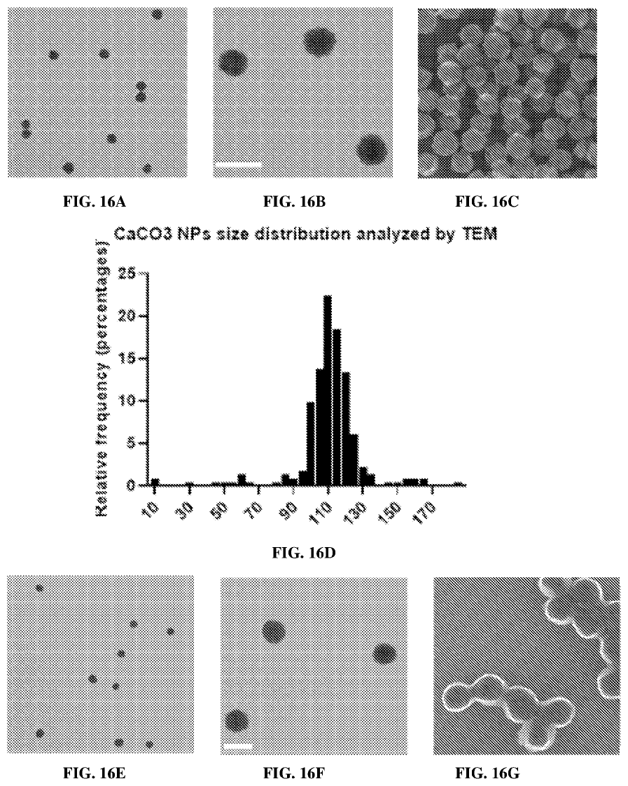

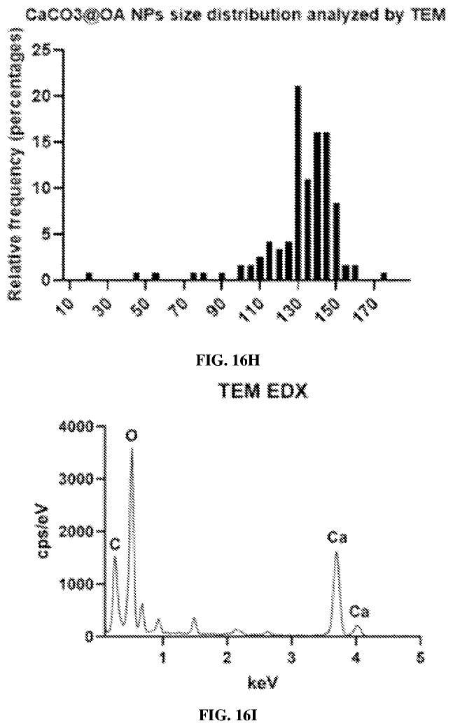

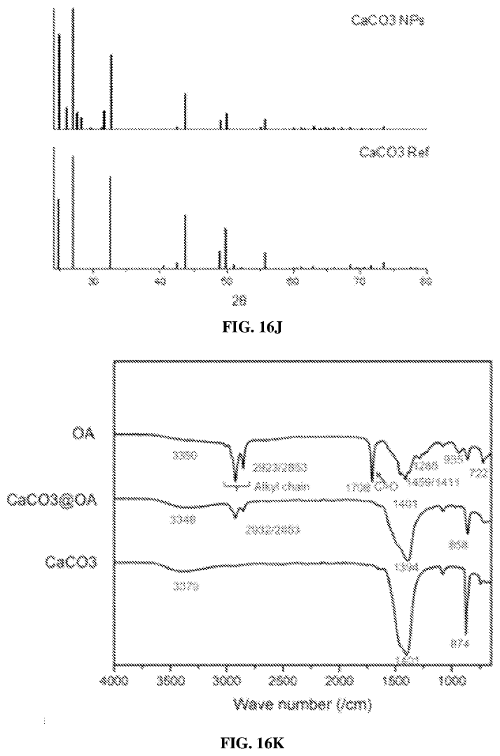

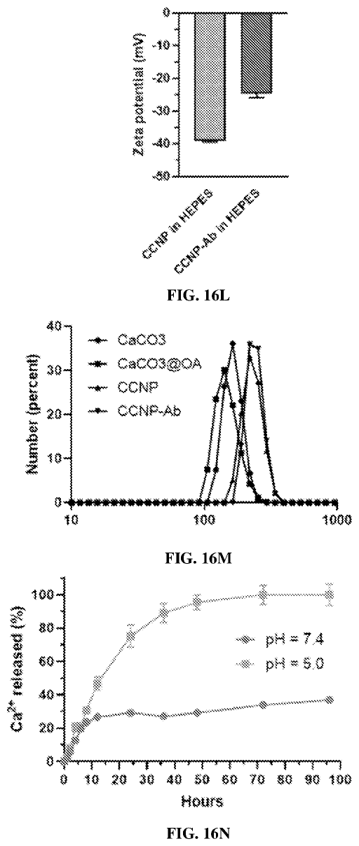

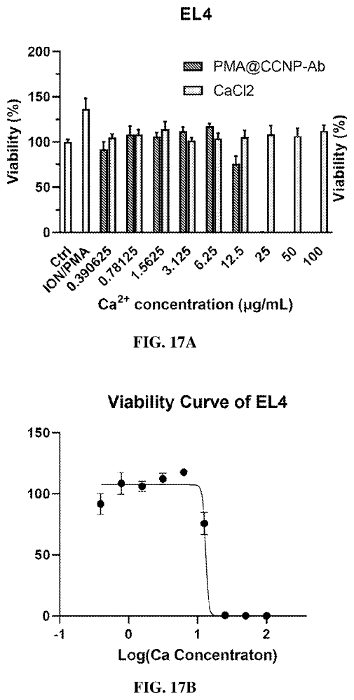

As a representative example, calcium carbonate (CaCO₃) nanoparticles loaded with PMA and surface-conjugated with anti-PD1 antibodies, termed PMA@CCNP-Ab were prepared. First, calcium carbonate (CaCO₃) nanoparticles were prepared by a co-precipitation method with calcium chloride and ammonium bicarbonate was used. Transmission electron microscopy (TEM) and scanning electron microscopy (SEM) found that the resulting nanoparticles exhibited good homogeneity (FIG. 16A-16D). The elemental composition of the CaCO₃ nanoparticles was investigated by energy dispersive X-ray spectroscopy (EDX), as shown in FIG. 16I. Peaks for calcium (Ca), carbon (C) and oxygen (O) were identified. X-ray diffraction (XRD) analysis (FIG. 16J) further confirmed that the nanoparticles were made of CaCO₃.

Next, CaCO₃ particles were coated with oleic acid to form CaCO₃@OA nanoparticles. The TEM and SEM images (FIGS. 16E, 16F, and 16G) revealed the morphology of CaCO₃@OA. The nanoparticle surfaces appeared significantly smoother. FIG. 16H shows the size distribution of CaCO₃@OA. Infrared (IR) spectroscopy measurements provided additional evidence of oleic acid conjugation on the CaCO₃ nanoparticles.

The aforementioned CaCO₃@OA nanoparticles are hydrophobic and can be dispersed in hexane. The CaCO₃@OA nanoparticles were coated with PEGylated phospholipids such as 1,2-distearoyl-sn-glycero-3-phosphoethanolamine-N-[carboxy(polyethylene glycol)-2000] (DSPE-PEG-COOH) so they could be more easily dispersed in aqueous solution.

Anti-PD1 antibodies were conjugated to CCNP using EDC/NHS chemistry. The decrease in zeta potential (FIG. 16L) after conjugation indicates successful antibody conjugation. Nanoparticle size was increased after lipid coating and antibody conjugation (FIG. 16M). When tested in solutions, it was discovered that Ca²⁺ was released slowly over 96 hours at pH 5.0 (FIG. 16N). In comparison, the release plateaued after 24 hours at neutral pH.

PMA was incorporated into CCNP-Ab to form PMA@CCNP-Ab.

### Cytotoxicity of CCNP

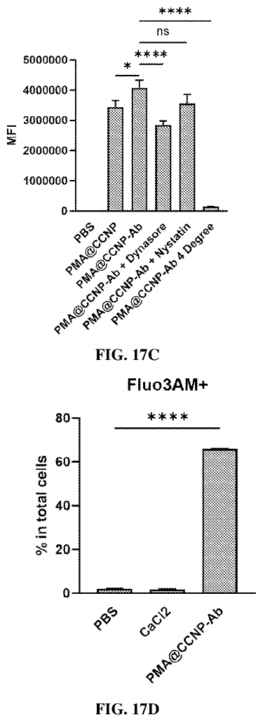

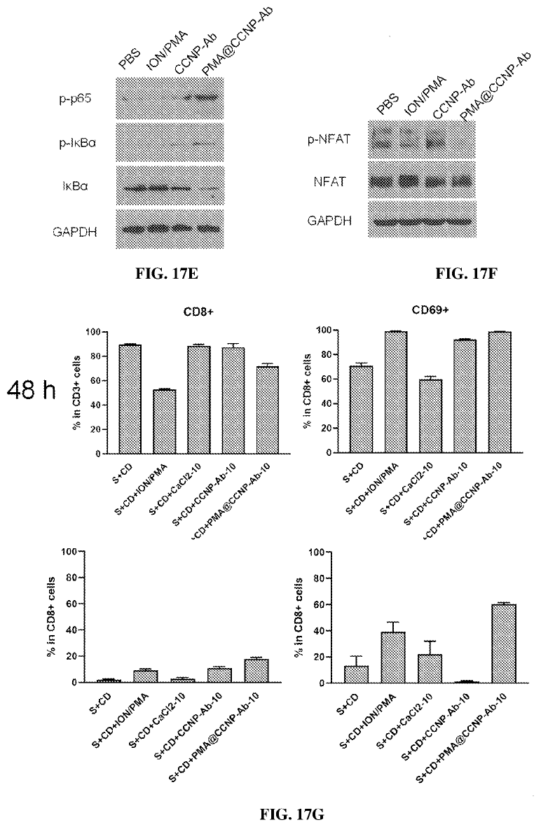

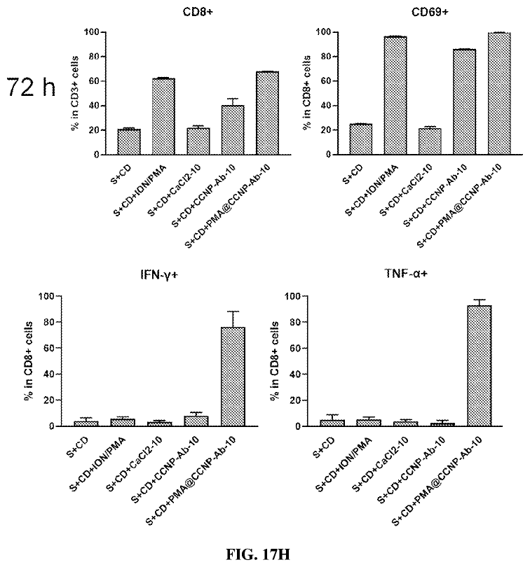

The cytotoxicity of PMA-loaded nanoparticles (PMA@CCNP-Ab) was evaluated using the EL4 cell line. PMA@CCNP-Ab were well tolerated by cells, but showed significant toxicity when the Ca²⁺ concentration was above 12.5 μg/mL (FIG. 17A). This may be due to calcium overload in T cells at higher concentrations. In comparison, CaCl₂) salt, due to its inability to penetrate the cell membrane, showed no significant effect on cell viability until the Ca²⁺ concentration reached 100 μg/mL (FIG. 17B). A calcium dose of 10 μg/mL was used in subsequent in vitro studies.

### Cellular Uptake and Effect on Cytosolic Calcium Levels

PMA@CCNP and PMA@CCNP-Ab were labelled with Cy5 and evaluated for intracellular uptake of the nanoparticles using EL4 cells, which are PD-1 positive. For comparison, the nanoparticles were also incubated with endocytosis inhibitors such as dynasore or nystatin. PMA@CCNP-Ab showed a significant increase in nanoparticle uptake compared to PMA@CCNP (FIG. 17C), which is attributed to PD-1-mediated endocytosis of PMA@CCNP-Ab. The uptake of PMA@CCNP-Ab was decreased when co-incubated with dynasore, indicating that the internalization of PMA@CCNP-Ab involves dynamin-dependent pathways. Conversely, nystatin did not inhibit cellular uptake, indicating that internalization is independent of lipid-mediated uptake processes. Incubation at 4° C. effectively reduced nanoparticle uptake, indicating that nanoparticle uptake is mediated by endocytosis rather than diffusion.

Changes in intracellular calcium levels were measured using Fluo-3AM as an indicator. PMA@CCNP-Ab significantly increased intracellular calcium levels (FIG. 17D), which is attributed to the degradation of the nanoparticles inside the cells and the resulting release of calcium. In comparison, CaCl₂) salt at the same calcium concentration did not increase cellular calcium levels.

The ability of PMA@CCNP-Ab to activate T cells was also evaluated by Western blotting. PMA@CCNP-Ab nanoparticles efficiently activated the NF-κB pathway as demonstrated by increased expression of phosphorylated p65 and IκBα (FIG. 17E). In addition, PMA@CCNP-Ab also activated the NFAT pathway, as evidenced by increased dephosphorylation of NFAT (FIG. 17F). Both pathways are known to be involved in calcium signaling for T cell activation.

### Effect on T Cell Activation

The effects of PMA@CCNP-Ab were evaluated on T cells derived from the spleen of OT-1 mice (FIGS. 17G, 17H). Cytotoxic T cells (CTLs) from OT-1 mice recognize OVA and are widely used as a tool to study antigen-specific immunity. OT-1 T cells were primed with anti-CD3 and anti-CD28 antibodies prior to incubation with PMA@CCNP-Ab. PBS, ION/PMA, CaCl₂) and CCNP-Ab were tested for comparison. After 48 or 72 hours, cells were harvested, stained for CD8, CD69, IFN-γ, and TNF-α, and analyzed by flow cytometry. The results showed that PMA@CCNP-Ab could significantly increase the population of CD69+ in OT-1 CTLs, and the efficacy was comparable to that of ION/PMA (FIG. 17G, 17H). The frequencies of IFN-γ- and TNF-α-positive CTLs were increased, further supporting T-cell activation. The stimulatory effects were more pronounced at 72 hours.

T cell activation was evaluated by analyzing cytokine release from OT-1 cells after incubation with PMA@CCNP-Ab. This was assessed by ELISA using a co-culture of OT-1 splenocytes and irradiated (100 Gy) B16-OVA cancer cells (FIGS. 17I, 17J). Results showed an increased secretion of IFN-γ and IL-2 when cells were incubated with PMA@CCNP-Ab. Taken together, these results indicate that PMA@CCNP-Ab nanoparticles are able to enhance the activation of T cells.

### Impact on Cellular Immunity In Vivo

B16-OVA cells were inoculated into C57BL/6 mice. When the tumor size reached 100 mm³ (Day 1), X-rays (15 Gy) was applied to trigger intratumoral immune response. On Day 2, 5 and 8, PMA@CCNP-Ab was injected intratumorally (i.t.) at a dose of 5 μg calcium and 10 ng PMA per mouse. PBS, ION/PMA, CaCl₂), and CCNP-Ab were injected i.t. for comparison. Non-irradiated mice were also examined. All mice were sacrificed on Day 13. Tumors, spleens, and lymph nodes were harvested, processed to single cells, and stained for CD45, CD3, CD8, CD4, IFN-γ, and FoxP3.

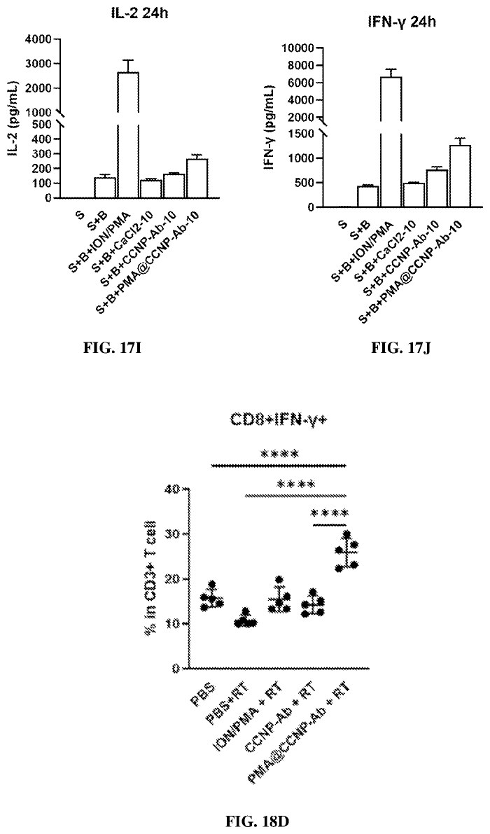

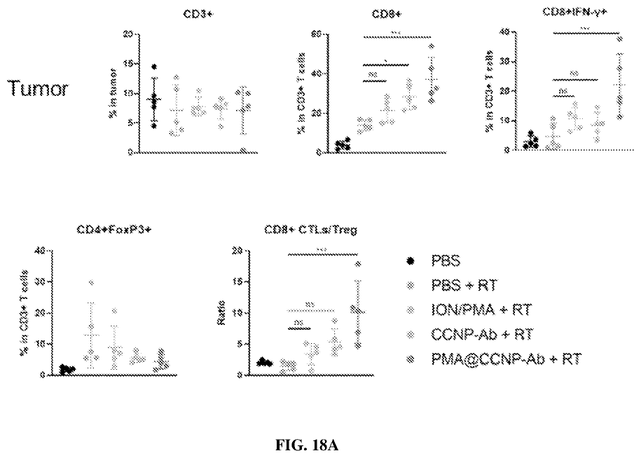

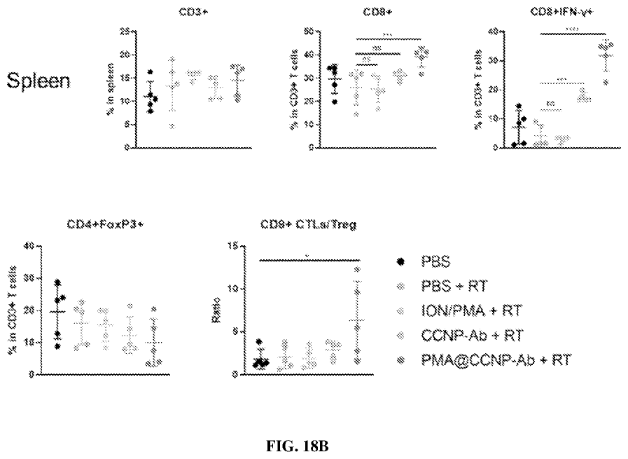

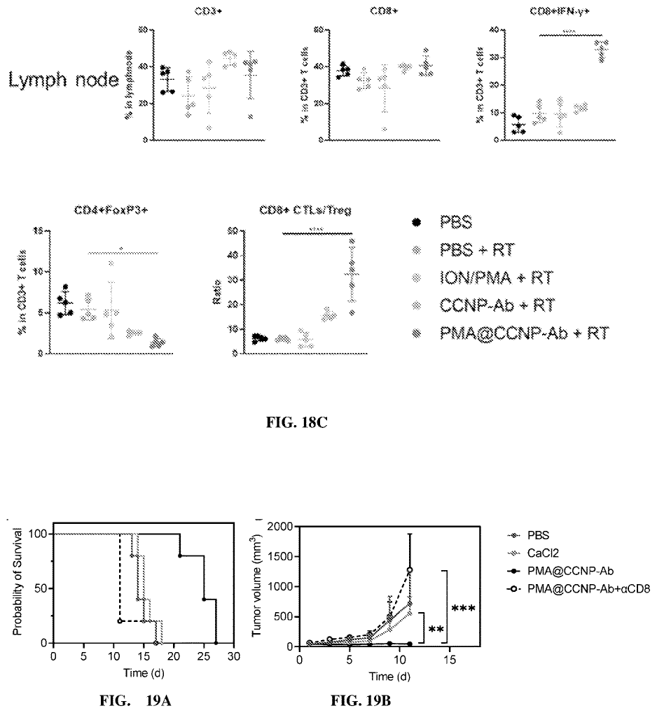

Flow cytometric analysis revealed increased infiltration of CD8₊ T cells (CTLs) into the tumor in the PMA@CCNP-Ab treated group (FIG. 18A). The tumor CTL/Treg ratio was also significantly higher in the PMA@CCNP-Ab group than in the other treatment groups. Similar patterns were observed in spleen and lymph nodes (FIGS. 18B, 18C). These results indicate that PMA@CCNP-Ab enhanced T-cell activation and proliferation, which in turn enhanced cellular immunity.

Splenocytes from different groups were cocultured with B16-OVA ex vivo to evaluate the effects of treatments on cellular immunity (FIG. 18D). A significantly increased number of activated CTLs (CD8+IFN-γ+) with splenocytes from the PMA@CCNP-Ab group were observed, confirming that the nanoparticles enhanced antigen-specific immune response against tumors.

### Therapy Efficacy

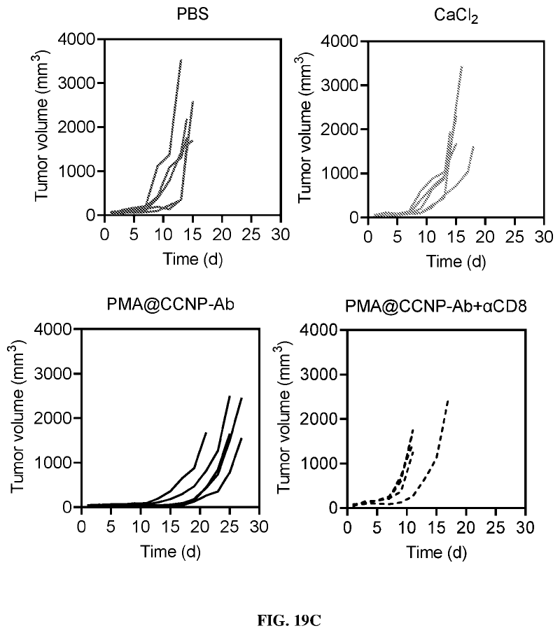

Next, the efficacy of PMA@CCNP-Ab was evaluated in B16 tumor-bearing C57BL/6 mice. PMA@CCNP-Ab nanoparticles were injected i.t. at a dose of 5 μg calcium and 10 ng PMA per mouse. A total of three doses were administered two days apart. For comparison, CaCl₂) salt was injected i.t. at the same calcium dose. PMA@CCNP-Ab effectively suppressed tumor growth and significantly improved animal survival (FIGS. 19A-19C). Meanwhile, when anti-CD8 antibodies, which deplete CTLs in animals, were injected, the therapeutic benefits were abolished (FIGS. 19A-19C), indicating that activation of cellular immunity was a major cause of tumor suppression with PMA@CCNP-Ab nanoparticles. No acute or chronic toxicities were observed in animals treated with PMA@CCNP-Ab.

Collectively, these results show that T cells efficiently internalized PMA@CCNP-Ab, resulting in increased intracellular calcium levels. Delivery of calcium and PMA to T cells promoted their activation as evidenced by increased expression or secretion of CD69, IFN-γ, and TNF-α. This was observed both in the EL4 cell line and in primary T cells from OT1 mice. In vivo testing in B16-OVA tumor-bearing C57/BL6 mice showed that PMA@CCNP-Ab resulted in enhanced tumor infiltration by cytotoxic T cells and increased CTL/Treg ratios. Therapeutic benefits associated with PMA@CCNP-Ab's ability to enhance T cell activation were observed. In addition, PMA@CCNP-Ab can be used to enhance cell-based therapies, including adoptive T-cell transfer and CAR-T therapies.

The disclosed nanotechnology offers several features that contribute to its uniqueness:

Controlled calcium release: T-cell activation requires a sustained increase in intracellular calcium concentration ([Ca²⁺]ᵢₙₜ). Achieving a sustained increase in [Ca²⁺]ᵢₙₜ is impossible with calcium salts (due to the ion-impermeable plasma membrane) or bare calcium nanoparticles (due to rapid particle dissolution in TME). To solve this problem, a lipid coating layer was used that prevents nanoparticles from rapid degradation, allowing nanoparticles to enter cells through endocytosis and gradually release calcium ions inside cells.

Low toxicity: Unlike cytokine or interferon-based immunomodulators, the calcium nanoparticles have low toxicity and can be administered repeatedly without causing systemic toxicity. After treatment, the nanoparticles degrade to Ca²⁺ and CO₃²⁻, which are safely excreted, metabolized or absorbed by the host.

Targeted delivery: The nanoparticles can be conjugated with T cell-targeting ligands, such as anti-PD1 or anti-CD3 antibodies, enabling targeted delivery of calcium and PKC antagonists to T cells. In comparison, conventional stimulation tools such as the ionomycin-PMA combination is effective in vitro but not in vivo due to rapid clearance and lack of specificity.

Unique mechanism of action: Typically, engagement of T cell receptors (TCRs) with antigens in the MHC-I context activates phospholipase Cγ1 (PLCγ1) and produces inositol 1,4,5-triphosphate (IP3). IP3 binds to its receptors on the endoplasmic reticulum (ER) membrane of T cells, resulting in calcium flux from the ER into the cytosol. Luminal calcium depletion is sensed by STIM1/2 and triggers their translocation to the plasma membrane, where they activate Orai1/2 to form a Ca²⁺-selective pore (i.e., CRAC channel) and induce Ca²⁺ influx (i.e., store-operated calcium entry, SOCE). Activation can be suppressed or blocked at multiple stages, dampening cellular immunity. In the disclosed approach, calcium delivery bypasses upstream signaling, which is believed to allow T cell activation even in immunosuppressive environments.

We claim:

1. A nanoparticle comprising a calcium core and a shell and/or a coating.

2. The nanoparticle of claim 1 wherein the core further comprises hydroxide, and optionally is calcium hydroxide (Ca(OH)₂).

3. The nanoparticle of claim 1, wherein the core further comprises carbonate, and optionally is calcium carbonate (CaCO₃).

4. The nanoparticle of claim 1, wherein the core is selected from calcium citrate (CaCit), calcium phosphate (Ca₃(PO₄)₂), CaCL₂, calcium sulfate (CaSO₄), CaC₂O₄, Ca(NO₃)₂, calcium silicate (Ca₂SiO₄), calcium fluoride (CaF₂), CaBr₂, and CaI₂.

5. The nanoparticle of any one of claims 1-4 comprising the shell.

6. The nanoparticle of claim 5, wherein the shell reduces, prevents, or otherwise delays degradation of the nanoparticle.

7. The nanoparticle of claim 5 or 6, wherein the shell comprises one or more of silica, mesoporous silica, carbon, a sulfide optionally ZnS, CoS, CuS, Cu2S, FeS, MoS, Al2S3, Y2S3, or MnS; an oxide optionally Fe3O4, Fe2O3, Gd2O3, TiO2, Al2O3, or MnO2; a fluoride optionally NaYF4, YF3, LaF3, CeF3, PrF3, or GdFe3; a fatty acid optionally oleic acid, myristic acid, palmitic acid, palmitoleic acid, stearic acid, oleic acid, linoleic acid, arachidic acid, eicosapentaenoic acid (EPA), or docosahexaenoic acid (DHA); an alkyl amine optionally octylamine, nonylamine, decylamine, undecylamine, laurylamine, tridecylamine, tetradecylamine, pentadecylamine, hexadecylamine, heptadecylamine, octadecylamine, oleylamine; MgO, CuO, or ZnO.

8. The nanoparticle of any one of claims 1-7, wherein the nanoparticle comprises the coating.

9. The nanoparticle of claim 8, wherein the coating improves dispersion in aqueous solutions and/or delays core release and/or improves half-life.

10. The nanoparticle of claims 8 and 9, wherein the coating comprises one or more polymers, peptides, proteins, lipids, or a combination thereof.

11. The nanoparticle of any one of claims 8-10, wherein the coating comprises PEG.

12. The nanoparticle of any one of claims 1-11 comprising a targeting agent optionally wherein the target agent targets one or more immune cells, optionally wherein the one or more immune cells is selected from dendritic cells, T cells, macrophages, natural killer cells, neutrophils, and combinations thereof optionally wherein the T cells are selected from cytotoxic, helper, regulatory, memory T cells, gamma-delta T cells (γδ T cells), follicular helper T cells (Tfh), natural killer T cells (NKT cells), and combinations thereof.

13. The nanoparticle of claim 12, wherein the target agent targets dendritic cells.

14. The nanoparticle of claims 12 and 13, wherein the target agent targets CD205 and optionally is an anti-CD205 antibody.

15. The nanoparticle of claim 12, wherein the targeting agent targets T cells.

16. The nanoparticle of claim 15, wherein the T cells comprise or are cytotoxic T cells.

17. The nanoparticle of claim 15 or 16, wherein the targeting agent targets CD3 or PD-1, and optionally is an anti-CD3 or anti-PD-1 antibody.

18. The nanoparticle of any one of claims 1-17, further comprising an active agent, optionally selected from an antigen, chemotherapeutic drug, immune system modulator, immune checkpoint modulator, or an immune cell modulator.

19. The nanoparticle of claim 18, comprising an immune cell modulator optionally wherein the immune cell modulator is a Protein Kinase C (PKC) antagonist optionally wherein the PKC antagonist is phorbol 12-myristate 13-acetate (PMA).

20. A pharmaceutical composition comprising the nanoparticles of any one of claims 1-19.

21. The pharmaceutical composition of claim 20 further comprising an adjuvant.

22. The pharmaceutical composition of claim 20 or 21 further comprising an antigen, chemotherapeutic drug, immune system modulator, immune checkpoint modulator, or immune cell modulator.

23. A pharmaceutical composition comprising immune cells treated in vitro or ex vivo with the nanoparticles of any one of claims 1-19 optionally wherein the immune cells are selected from dendritic cells, T cells, macrophages, natural killer cells, neutrophils, and combinations thereof optionally wherein the T cells are selected from cytotoxic, helper, regulatory, memory T cells, gamma-delta T cells (γδ T cells), follicular helper T cells (Tfh), natural killer T cells (NKT cells), and combinations thereof.

24. A method of increasing calcium signaling in an immune cell comprising contacting the immune cell with an effective amount of the pharmaceutical composition of any one of claims 20-22 to increase calcium signaling therein, optionally wherein the immune cells is selected from dendritic cells, T cells, macrophages, natural killer cells, neutrophils, and combinations thereof, optionally wherein the T cells are selected from cytotoxic, helper, regulatory, memory T cells, gamma-delta T cells (γδ T cells), follicular helper T cells (Tfh), natural killer T cells (NKT cells), and combinations thereof.

25. A method of enhancing an immune response in a subject in need thereof comprising administrating the subject an effective amount of the pharmaceutical composition of any one of claims 20-23.

26. The method of claim 25, wherein the immune response comprises one of more of increasing NF-κB signaling and/or cytokine activity in dendritic cells, improved dendritic cell infiltration into a tumor site, and/or improved dendritic cell maturation.

27. The method of claim 25 or 26, wherein the immune response comprises one or more of inducing dendritic cells to express or secrete chemokines (e.g. CXCL-1, CCL5, CXCL2 and/or CXCL10), cytokines (e.g. IL-1β, IL-12, and/or IL-6), or a combination thereof.

28. The method of claim 26 or 27, wherein the immune response comprises one or more of increased T cell activation, increased T cell localization to a tumor site, increased expression and/or secretion of CD69, IFN-γ, and/or TNF-α by T cells.

29. The method of any one of claims 24-28, wherein the subject has cancer or an infection.

30. A method of treating or preventing cancer comprising administering a subject in need thereof an effective amount of the pharmaceutical composition of any one of claims 20-23.

31. The method of claim 30, wherein the amount or means of administration is effective to induce an immune response against the cancer but not to have a direct anticancer effect.

32. A method of treating or preventing an infection comprising administering a subject in need thereof an effective amount of the pharmaceutical composition of any one of claims 20-23.

33. The method of any one of claims 25-32, further comprising treating the subject with one or more of surgery, radiotherapy, chemotherapy, or immunotherapy optionally an immune checkpoint modulator, an immune system modulator, or immune cell modulator.


SEQUENCE LISTING (10 sequences)

SEQ ID NO: 1 - DNA, 20 bp, synthetic construct
  source 1..20: mol_type = other DNA
agagccacagtcctctttgc

SEQ ID NO: 2 - DNA, 20 bp, synthetic construct
  source 1..20: mol_type = other DNA
gctcctcttccaaggtgctt

SEQ ID NO: 3 - DNA, 20 bp, synthetic construct
  source 1..20: mol_type = other DNA
ctgctgctttgcctacctct

SEQ ID NO: 4 - DNA, 20 bp, synthetic construct
  source 1..20: mol_type = other DNA
cgagtgacaaacacgactgc

SEQ ID NO: 5 - DNA, 23 bp, synthetic construct
  source 1..23: mol_type = other DNA
ctgggattcacctcaagaacatc

SEQ ID NO: 6 - DNA, 18 bp, synthetic construct
  source 1..18: mol_type = other DNA
cagggtcaggcaagcctc

SEQ ID NO: 7 - DNA, 23 bp, synthetic construct
  source 1..23: mol_type = other DNA
atgagaactacagcaccagcttc

SEQ ID NO: 8 - DNA, 23 bp, synthetic construct
  source 1..23: mol_type = other DNA
acttgagggagaagtaggaatgg

SEQ ID NO: 9 - DNA, 20 bp, synthetic construct
  source 1..20: mol_type = other DNA
tcgtgctgtcggacccatat

SEQ ID NO: 10 - DNA, 22 bp, synthetic construct
  source 1..22: mol_type = other DNA
gtcgttgcttggttctccttgt